(19) United States
(12) Patent Application Publication
(10) Pub. No.: US 2025/0154602 A1
(43) Pub. Date: May 15, 2025

(54) COMPOSITIONS AND METHODS FOR DETECTING RARE SEQUENCE VARIANTS

(71) Applicant: AccuraGen Holdings Limited, Georgetown (KY)

(72) Inventors: Li WENG, Fremont, CA (US); Tobias WITTKOP, San Mateo, CA (US); Malek FAHAM, Burlingame, CA (US)

(21) Appl. No.: 18/955,777

(22) Filed: Nov. 21, 2024

Related U.S. Application Data
(63) Continuation of application No. PCT/US2023/023113, filed on May 22, 2023.
(60) Provisional application No. 63/345,364, filed on May 24, 2022.

(51) Int. Cl. C12Q 1/6886; C12Q 1/682; C12Q 1/6874

(52) U.S. Cl. CPC C12Q 1/6886; C12Q 1/682; C12Q 1/6874; C12Q 2600/156

(57) ABSTRACT

Provided herein are methods and compositions for nucleic acid analysis, in particular methods for identifying sequence variants.

### CROSS-REFERENCE

This application is a continuation of PCT International Application No. PCT/US2023/023113, filed May 22, 2023, which claims the benefit of U.S. Provisional Application No. 63/345,364, filed May 24, 2022, each of which is incorporated herein by reference in its entirety.

### BACKGROUND

Identifying sequence variation in nucleic acid populations, such as cell-free nucleic acids, is an actively growing field, particularly with the advent of large scale parallel nucleic acid sequencing. However, large scale parallel sequencing has significant limitations in that the inherent error frequency in commonly-used techniques is larger than the frequency of many of the actual sequence variations in the population. For example, error rates of 0.1-1% have been reported in standard high throughput sequencing. Detection of rare sequence variants has high false positive rates when the frequency of variants is low, such as at or below the error rate.

Rare variant detection can also be important for the early detection of pathological mutations. For instance, detection of cancer-associated point mutations in clinical samples can improve the identification of minimal residual disease during chemotherapy and detect the appearance of tumor cells in relapsing patients. The detection of rare point mutations is also important for the assessment of exposure to environmental mutagens, to monitor endogenous DNA repair, and to study the accumulation of somatic mutations in aging individuals. Additionally, more sensitive methods to detect rare variants can enhance prenatal diagnosis, enabling the characterization of fetal cells present in maternal blood.

### SUMMARY

In view of the foregoing, there is a need for improved methods of detecting rare sequence variants. The compositions and methods of the present disclosure address this need, and provide additional advantages as well. In particular, the various aspects of the disclosure provide for highly sensitive detection of rare or low frequency nucleic acid sequence variants (sometimes referred to as mutations). This includes identification and elucidation of low frequency nucleic acid variations (including substitutions, insertions, and deletions) in samples that may contain low amounts of variant sequences in a background of normal sequences, as well as the identification of low frequency variations in a background of sequencing errors.

In an aspect, provided herein are methods of identifying a sequence variant. In some embodiments methods comprise circularizing individual polynucleotides of a plurality of polynucleotides to form a plurality of circular polynucleotides, each circular polynucleotide having a junction between a 5′ end and a 3′ end. In some embodiments, the method comprises amplifying the plurality of circular polynucleotides to produce a plurality of amplified polynucleotides, each amplified polynucleotide having more than one copy of the circular polynucleotide. In some embodiments, the method comprises shearing the amplified polynucleotides or a derivative thereof to produce a plurality of sheared polynucleotides, each sheared polynucleotide comprising a 5′ end shear point and a 3′ end shear point. In some embodiments, the method comprises subjecting the plurality of sheared polynucleotides or a derivative thereof to sequencing to identify a plurality of sequence reads of the plurality of sheared polynucleotides. In some embodiments, the method comprise comparing the plurality of sequence reads to a reference sequence to obtain a sequence difference. In some embodiments, the method comprises calling a sequence difference as the sequence variant when the sequence difference occurs in at least two copies on one sheared polynucleotide and at least two different sheared polynucleotides having different 5′ end shear points and/or 3′ end shear points.

In various aspects of methods herein, the method further comprises attaching a first adapter to the 5′ end shear point and a second adapter to the 3′ end shear point of each of the plurality of sheared polynucleotides or a derivative thereof to create a plurality of adapter-linked sheared polynucleotides. In some embodiments, the method further comprises amplifying the plurality of adapter-linked sheared polynucleotides using a first primer that binds to the first adapter and a second primer that binds to the second adapter. In some embodiments, the method further comprises amplifying one or more target sequences of the plurality of adapter-linked sheared polynucleotides using a first primer that binds to the first adapter and at least a second primer that binds to the one or more target sequences in the sheared polynucleotide.

In some embodiments, the method further comprises enriching a target sequence in the plurality of sheared polynucleotides or a derivative thereof. In some embodiments, enriching comprises contacting the plurality of sheared polynucleotides or a derivative thereof with a capture probe that binds to the target sequence. In some embodiments, enriching comprises amplification with at least one primer that binds to the target sequence.

In various aspects of methods herein, circularizing comprises ligating ends of each of the plurality of polynucleotides or a derivative thereof to one another. Alternatively, circularizing comprises coupling an adapter to the 5′ end, the 5′ end, or both the 5′ end and the 3′ end of each of the plurality of polynucleotides or a derivative thereof.

In some embodiments, amplifying is effected by a polymerase having strand-displacement activity. In some embodiments, amplifying is effected by a polymerase having 5′ to 3′ exonuclease activity. In some embodiments, amplifying comprises contacting the plurality of circular polynucleotides with an amplification reaction mixture comprising random primers. In some embodiments, amplifying comprises contacting the plurality of circular polynucleotides with an amplification mixture comprising at least one primer that hybridizes to a target sequence of at least one of the plurality of circular polynucleotides.

In various aspects of methods herein, the polynucleotides are single-stranded. Alternatively, or in combination, the polynucleotides are double-stranded. In some embodiments, the polynucleotides are cell-free polynucleotides. In some embodiments, the polynucleotides are deoxyribonucleic acid (DNA), ribonucleic acid (RNA), or a combination thereof. In some embodiments, the polynucleotides are from a tumor.

In various aspects of methods herein, sequencing comprises bringing the plurality of sheared polynucleotides or a derivative thereof in contact with a plurality of nucleotides in the presence of a polymerase to incorporate one or more nucleotides of the plurality of nucleotides into a growing strand complementary to a strand of the sheared polynucleotides or derivative thereof, and detecting one or more signals indicative of incorporation of the one or more nucleotides into the growing strand. In some embodiments, sequencing comprises sequencing by ligation. In some embodiments, the sequence variant comprises a single nucleotide variant, a fusion, an insertion, a deletion, or an epigenetic modification. In some embodiments, the polynucleotides are from a bodily fluid. In some embodiments, the bodily fluid comprises urine, saliva, blood, serum, or plasma. In some embodiments, the variant is indicative of minimum residual disease (MRD). In some embodiments, the method comprises detecting MRD.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example workflow for variant detection.

FIG. 2 shows an example workflow for polynucleotide amplification.

FIG. 3 shows an example workflow for polynucleotide amplification.

FIG. 4 shows an example workflow for targeted polynucleotide amplification.

FIG. 5 shows an example workflow for polynucleotide amplification.

FIG. 6 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

In one aspect, provided herein are methods of identifying a sequence variant. In some embodiments, the method comprises circularizing individual polynucleotides of a plurality of polynucleotides to form a plurality of circular polynucleotides, each circular polynucleotide having a junction between a 5′ end and a 3′ end. Next the method can comprise amplifying the plurality of circular polynucleotides to produce a plurality of amplified polynucleotides, each amplified polynucleotide having more than one copy of the circular polynucleotide. Then the method can comprise shearing the amplified polynucleotides or a derivative thereof to produce a plurality of sheared polynucleotides, each sheared polynucleotide comprising a 5′ end shear point and a 3′ end shear point. Next the method can comprise subjecting the plurality of sheared polynucleotides or a derivative thereof to sequencing to identify a plurality of sequence reads of the plurality of sheared polynucleotides. The method can then comprise comparing the plurality of sequence reads to a reference sequence to obtain a sequence difference. Next the method can comprise calling a sequence difference as the sequence variant when the sequence difference occurs in at least two copies on one sheared polynucleotide and at least two different sheared polynucleotides having different 5′ end shear points and/or 3′ end shear points.

In some embodiments, the method further comprises attaching a first adapter to the 5′ end shear point and a second adapter to the 3′ end shear point of each of the plurality of sheared polynucleotides or a derivative thereof to create a plurality of adapter-linked sheared polynucleotides. In some embodiments, the method further comprises amplifying the plurality of adapter-linked sheared polynucleotides or a derivative thereof using a first primer that binds to the first adapter and a second primer that binds to the second adapter. In some embodiments, the method further comprises amplifying one or more target sequences of the plurality of adapter-linked sheared polynucleotides or a derivative thereof using a first primer that binds to the first adapter and at least a second primer that binds to the one or more target sequences in the sheared polynucleotide. In some embodiments, the method further comprises a second amplification step of the one or more target sequences using a third primer and a fourth primer. In some embodiments, the third primer and the fourth primer are nested primers.

In some cases, the method further comprises enriching a target sequence in the plurality of sheared polynucleotides. In some embodiments, enriching comprises contacting the plurality of sheared polynucleotides or a derivative thereof with a capture probe that binds to the target sequence. In some embodiments, enriching comprises amplification with at least one primer that binds to the target sequence. In some embodiments, enriching comprises any suitable enrichment method provided herein. In some embodiments, enriching comprises a second amplification step using nested primers.

In some cases, circularization comprises ligating ends of each of the plurality of polynucleotides or a derivative thereof to one another. In some cases, circularization comprises coupling an adapter to the 5′ end, the 5′ end, or both the 5′ end and the 3′ end of each of the plurality of polynucleotides or a derivative thereof. In some embodiments, circularization comprises any suitable circularization method provided herein.

Amplification of circularized polynucleotides can be effected by a polymerase having strand-displacement activity. In some embodiments, amplification is effected by a polymerase having 5′ to 3′ exonuclease activity. In some embodiments, amplification comprises contacting the plurality of circular polynucleotides with an amplification reaction mixture comprising random primers. In some embodiments, amplification comprises contacting the plurality of circular polynucleotides with an amplification mixture comprising at least one primer that hybridizes to a target sequence of at least one of the plurality of circular polynucleotides. In some embodiments, amplification is effected using any suitable method provided herein.

In various aspects of method provided herein, in some cases the polynucleotides are single-stranded. In some cases, the polynucleotides are double-stranded. In some cases, the polynucleotides are cell-free polynucleotides. In some cases, the polynucleotides are deoxyribonucleic acid (DNA), ribonucleic acid (RNA), or a combination thereof. In some cases, the polynucleotides are from a tumor. In some embodiments, the method comprises detecting minimum residual disease (MRD).

In various aspects of methods herein sequencing can comprise bringing the plurality of sheared polynucleotides or a derivative thereof in contact with a plurality of nucleotides in the presence of a polymerase to incorporate one or more nucleotides of the plurality of nucleotides into a growing strand complementary to a strand of the sheared polynucleotides or derivative thereof, and detecting one or more signals indicative of incorporation of the one or more nucleotides into the growing strand. In some cases, sequencing comprises sequencing by ligation. In some cases, sequencing comprises any suitable method provided herein.

In various aspects of methods herein the sequence variant comprises a single nucleotide variant, a fusion, an insertion, a deletion, or an epigenetic modification. In some embodiments, the sequence variant is indicative of MRD.

In various aspects of methods herein the polynucleotides are from a bodily fluid. In some cases, the bodily fluid comprises urine, saliva, blood, serum, or plasma. In some cases, the polynucleotides are from any suitable source provided herein.

In one aspect, the disclosure provides a method of identifying a sequence variant comprising (a) circularizing individual polynucleotides of a plurality of polynucleotides to form a plurality of circular polynucleotides, each circular polynucleotide having a junction between a 5′ end and a 3′ end; (b) amplifying said plurality of circular polynucleotides of (a) to produce a plurality of amplified polynucleotides, each amplified polynucleotide having more than one copy of the circular polynucleotide; (c) shearing said amplified polynucleotides or a derivative thereof to produce a plurality of sheared polynucleotides, each sheared polynucleotide comprising a 5′ end shear point and a 3′ end shear point; (d) subjecting said plurality of sheared polynucleotides or a derivative thereof to sequencing to identify a plurality of sequence reads of said plurality of sheared polynucleotides; (e) comparing said plurality of sequence reads to a reference sequence to obtain a sequence difference; and (f) calling a sequence difference as the sequence variant when the sequence difference occurs in (i) at least two copies on one sheared polynucleotide or (ii) at least two different sheared polynucleotides having different 5′ end shear points and/or 3′ end shear points.

In some embodiments, the method further comprises attaching a first adapter to the 5′ end shear point and a second adapter to the 3′ end shear point of each of the plurality of sheared polynucleotides or a derivative thereof to create a plurality of adapter-linked sheared polynucleotides. In some embodiments, the method further comprises amplifying the plurality of adapter-linked sheared polynucleotides or a derivative thereof using a first primer that binds to the first adapter and a second primer that binds to the second adapter. In some embodiments, the method further comprises amplifying one or more target sequences of the plurality of adapter-linked sheared polynucleotides or a derivative thereof using a first primer that binds to the first adapter and at least a second primer that binds to the one or more target sequences in the sheared polynucleotide. In some embodiments, the method further comprises a second amplification step of the one or more target sequences using a third primer and a fourth primer. In some embodiments, the third primer and the fourth primer are nested primers.

In some cases, the method further comprises enriching a target sequence in the plurality of sheared polynucleotides. In some embodiments, enriching comprises contacting the plurality of sheared polynucleotides or a derivative thereof with a capture probe that binds to the target sequence. In some embodiments, enriching comprises amplification with at least one primer that binds to the target sequence. In some embodiments, enriching comprises any suitable enrichment method provided herein. In some embodiments, enriching comprises a second amplification step using nested primers.

In some cases, circularization comprises ligating ends of each of the plurality of polynucleotides or a derivative thereof to one another. In some cases, circularization comprises coupling an adapter to the 5′ end, the 5′ end, or both the 5′ end and the 3′ end of each of the plurality of polynucleotides or a derivative thereof. In some embodiments, circularization comprises any suitable circularization method provided herein.

Amplification of circularized polynucleotides can be effected by a polymerase having strand-displacement activity. In some embodiments, amplification is effected by a polymerase having 5′ to 3′ exonuclease activity. In some embodiments, amplification comprises contacting the plurality of circular polynucleotides with an amplification reaction mixture comprising random primers. In some embodiments, amplification comprises contacting the plurality of circular polynucleotides with an amplification mixture comprising at least one primer that hybridizes to a target sequence of at least one of the plurality of circular polynucleotides. In some embodiments, amplification is effected using any suitable method provided herein.

In various aspects of method provided herein, in some cases the polynucleotides are single-stranded. In some cases, the polynucleotides are double-stranded. In some cases, the polynucleotides are cell-free polynucleotides. In some cases, the polynucleotides are deoxyribonucleic acid (DNA), ribonucleic acid (RNA), or a combination thereof. In some cases, the polynucleotides are from a tumor. In some embodiments, the method comprises detecting minimum residual disease (MRD).

In various aspects of methods herein sequencing can comprise bringing the plurality of sheared polynucleotides or a derivative thereof in contact with a plurality of nucleotides in the presence of a polymerase to incorporate one or more nucleotides of the plurality of nucleotides into a growing strand complementary to a strand of the sheared polynucleotides or derivative thereof, and detecting one or more signals indicative of incorporation of the one or more nucleotides into the growing strand. In some cases, sequencing comprises sequencing by ligation. In some cases, sequencing comprises any suitable method provided herein.

In various aspects of methods herein the sequence variant comprises a single nucleotide variant, a fusion, an insertion, a deletion, or an epigenetic modification. In some embodiments, the sequence variant is indicative of MRD.

In various aspects of methods herein the polynucleotides are from a bodily fluid. In some cases, the bodily fluid comprises urine, saliva, blood, serum, or plasma. In some cases, the polynucleotides are from any suitable source provided herein.

In one aspect, the disclosure provides a method of identifying a sequence variant comprising (a) circularizing individual polynucleotides of a plurality of polynucleotides to form a plurality of circular polynucleotides, each circular polynucleotide having a junction between a 5′ end and a 3′ end; (b) amplifying said plurality of circular polynucleotides of (a) to produce a plurality of amplified polynucleotides, each amplified polynucleotide having more than one copy of the circular polynucleotide; (c) shearing said amplified polynucleotides or a derivative thereof to produce a plurality of sheared polynucleotides, each sheared polynucleotide comprising a 5′ end shear point and a 3′ end shear point; (d) subjecting said plurality of sheared polynucleotides or a derivative thereof to sequencing to identify a plurality of sequence reads of said plurality of sheared polynucleotides; (e) comparing said plurality of sequence reads to a reference sequence to obtain a sequence difference; and (f) calling a sequence difference as the sequence variant when the sequence difference occurs in at least two copies on one sheared polynucleotide.

In one aspect, the disclosure provides a method of identifying a sequence variant comprising (a) circularizing individual polynucleotides of a plurality of polynucleotides to form a plurality of circular polynucleotides, each circular polynucleotide having a junction between a 5′ end and a 3′ end; (b) amplifying said plurality of circular polynucleotides of (a) to produce a plurality of amplified polynucleotides, each amplified polynucleotide having more than one copy of the circular polynucleotide; (c) shearing said amplified polynucleotides or a derivative thereof to produce a plurality of sheared polynucleotides, each sheared polynucleotide comprising a 5′ end shear point and a 3′ end shear point; (d) subjecting said plurality of sheared polynucleotides or a derivative thereof to sequencing to identify a plurality of sequence reads of said plurality of sheared polynucleotides; (e) comparing said plurality of sequence reads to a reference sequence to obtain a sequence difference; and (f) calling a sequence difference as the sequence variant when the sequence difference occurs in at least two different sheared polynucleotides having different 5′ end shear points and/or 3′ end shear points.

In one aspect, the disclosure provides a method of identifying a sequence variant in a nucleic acid sample comprising a plurality of polynucleotides, each polynucleotide of the plurality having a 5′ end and a 3′ end. In some cases, the method comprises (a) circularizing individual polynucleotides of the plurality to form a plurality of circular polynucleotides, wherein a given circular polynucleotide of the plurality has a junction sequence resulting from said circularization; (b) amplifying the circularized polynucleotides of (a) to produce a plurality of amplified polynucleotides; (c) shearing the amplified polynucleotides to produce sheared polynucleotides, each sheared polynucleotide comprising one or more shear points at a 5′ end and/or a 3′ end; (d) sequencing the sheared polynucleotides and/or amplification products of the sheared polynucleotides to produce a plurality of sequencing reads; and (d) calling a sequence difference detected in the sequencing reads as the sequence variant when the sequence difference occurs in sequencing reads corresponding to a first sheared polynucleotide and a second sheared polynucleotide.

In some cases, the method comprises (a) circularizing individual polynucleotides of said plurality to form a plurality of circular polynucleotides, each of which having a junction between the 5′ end and the 3′ end; (b) amplifying the circular polynucleotides of (a) to produce amplified polynucleotides; (c) shearing the amplified polynucleotides to produce sheared polynucleotides, each sheared polynucleotide comprising one or more shear points at a 5′ end and/or 3′ end; (d) sequencing the sheared polynucleotides to produce a plurality of sequencing reads; (e) identifying sequencing differences between sequencing reads and a reference sequence; and (f) calling a sequence difference as the sequence variant when the sequence difference occurs in at least two different sheared polynucleotides.

In general, joining ends of a polynucleotide to one-another to form a circular polynucleotide (either directly, or with one or more intermediate adapter oligonucleotides) produces a junction having a junction sequence. Where the 5′ end and 3′ end of a polynucleotide are joined via an adapter polynucleotide, the term “junction” can refer to a junction between the polynucleotide and the adapter (e.g. one of the 5′ end junction or the 3′ end junction), or to the junction between the 5′ end and the 3′ end of the polynucleotide as formed by and including the adapter polynucleotide. Where the 5′ end and the 3′ end of a polynucleotide are joined without an intervening adapter (e.g. the 5′ end and 3′ end of a single-stranded DNA), the term “junction” refers to the point at which these two ends are joined. A junction may be identified by the sequence of nucleotides comprising the junction (also referred to as the “junction sequence”).

In some embodiments, samples comprise polynucleotides having a mixture of ends formed by natural degradation processes (such as cell lysis, cell death, and other processes by which polynucleotides such as DNA and RNA are released from a cell to its surrounding environment in which it may be further degraded, e.g., cell-free polynucleotides, e.g., cell-free DNA and cell-free RNA), fragmentation that is a byproduct of sample processing (such as fixing, staining, and/or storage procedures), and fragmentation by methods that cleave DNA without restriction to specific target sequences (e.g. mechanical fragmentation, such as by sonication; non-sequence specific nuclease treatment, such as DNase I, fragmentase). Where samples comprise polynucleotides having a mixture of ends, the likelihood of two polynucleotides having the same 5′ end or 3′ end is low, and the likelihood that two polynucleotides will independently have both the same 5′ end and 3′ end is lower. Accordingly, in some embodiments, junctions may be used to distinguish different polynucleotides, even where the two polynucleotides comprise a portion having the same target sequence. Where polynucleotide ends are joined without an intervening adapter, a junction sequence may be identified by alignment to a reference sequence. For example, where the order of two component sequences appears to be reversed with respect to the reference sequence, the point at which the reversal appears to occur may be an indication of a junction at that point. Where polynucleotide ends are joined via one or more adapter sequences, a junction may be identified by proximity to the known adapter sequence, or by alignment as above if a sequencing read is of sufficient length to obtain sequence from both the 5′ and 3′ ends of the circularized polynucleotide. In some embodiments, the formation of a particular junction is a sufficiently rare event such that it is unique among the circularized polynucleotides of a sample.

In some embodiments, circularizing individual polynucleotides in (a) is effected by subjected the plurality of polynucleotides to a ligation reaction. The ligation reaction may comprise a ligase enzyme. In some embodiments, the ligase enzyme is degraded prior to amplifying in (b). Degradation of ligase prior to amplifying in (b) can increase the recovery rate of amplifiable polynucleotides. In some embodiments, the plurality of circularized polynucleotides are not purified or isolated prior to (b). In some embodiments, uncircularized, linear polynucleotides are degraded prior to amplifying.

In some cases, circularizing in (a) comprises the step of joining and adapter polynucleotide to the 5′ end, the 3′ end, or both the 5′ end and the 3′ end of a polynucleotide in the plurality of polynucleotides. As previously described, where the 5′ end and/or 3′ end of a polynucleotide are joined via an adapter polynucleotide, the term “junction” can refer to the junction between the polynucleotide and the adapter (e.g., one of the 5′ end junction or the 3′ end junction), or to the junction between the 5′ end and the 3′ end of the polynucleotide as formed by and including the adapter polynucleotide.

The circularized polynucleotides can be amplified, for example, after degradation of the ligase enzyme, to yield amplified polynucleotides. Amplifying the circular polynucleotides in (b) can be effected by a polymerase having strand-displacement activity. In some cases, the polymerase is a Phi29 DNA polymerase. In some cases, amplification comprises rolling circle amplification (RCA). The amplified polynucleotides resulting from RCA can comprise linear concatemers, or polynucleotides comprising two or more copies of a target sequence (e.g., subunit sequence) from a template polynucleotide. In some embodiments, amplifying comprises subjecting the circular polynucleotides to an amplification reaction mixture comprising random primers. In some cases, amplifying comprises subjecting the circular polynucleotides to an amplification reaction mixture comprising one or more primers, each of which specifically hybridizes to a different target sequence via sequence complementarity.

The amplified polynucleotides are sheared, in some cases, to produce sheared polynucleotides that are shorter in length relative to the unsheared polynucleotides. Two or more sheared polynucleotides originating from the same linear concatemer may have the same junction sequence but can have different 5′ and/or 3′ ends (e.g., shear ends).

Amplified polynucleotides can be sheared using any variety of methods, such as, but not limited to, physical fragmentation, enzymatic methods, and chemical fragmentation. Non-limiting examples of physical fragmentation methods that can be employed for the fragmentation of amplified polynucleotides include acoustic shearing, sonication, and hydrodynamic shearing. In some cases, acoustic shearing and sonication may be preferred. Non-limiting examples of enzymatic fragmentation methods that can be employed for the fragmentation of amplified polynucleotides include use of enzymes such as DNase I and other restriction endonucleases, including non-specific nucleases, and transposases. Non-limiting examples of chemical fragmentation methods that can be employed for the fragmentation of amplified polynucleotides include use of heat and divalent metal cations.

Sheared polynucleotides (also referred to as fragmented polynucleotides) which are shorter in length compared to the unsheared polynucleotides may be desired to match the capabilities of the sequencing instrument used for producing sequencing reads, also referred to as sequence reads. For example, amplified polynucleotides may be fragmented, for example sheared, to the optimal length determined by the downstream sequencing platform. Various sequencing instruments, further described herein, can accommodate nucleic acids of different lengths. In some cases, amplified polynucleotides are sheared in the process of attaching adapters useful in downstream sequencing platforms, for example in flow cell attachment or sequencing primer binding. In some cases, sheared polynucleotides are subject to amplification to produce amplification products of the sheared polynucleotides prior to sequencing. Additional amplification can be desirable, for example, to generate a sufficient amount of polynucleotides for downstream analysis, for example, sequencing analysis. The resulting amplification products can comprise multiple copies of individual sheared polynucleotides.

During sequencing, sheared polynucleotides or amplification products thereof originating from the same amplified polynucleotide can be sequenced. Sequencing reads resulting from sequencing can be grouped into read families. A read family can comprise any suitable number of sequence reads. In some cases, a read family comprises at least 5, 10, 15, 20, 25, 50, 75, or 100 sequences reads. In some cases, a group of sequence reads may not be identified as a read family unless a minimum number of sequence reads are present. For example, a read family can comprise at least 2, 3, 4, 5, 7, 8, 9, or 10 sequence reads. In some cases, a read family comprises at least 25 read sequences. In some cases, sequence reads which may be classified as a read family based on a shared junction sequence and shared sequences of the 5′ and 3′ ends. In some embodiments, the sequence reads of a read family have the same junction sequence. In some embodiments, the sequence reads of a read family have the same sequences at the 5′ and 3′ end, for example, the sequences may be identical over at least 5 bases, 6 bases, 7 bases, 8 bases, 9 bases, or 10 bases at each of the 5′ and 3′ ends. In some cases, the sequences at the 5′ and 3′ ends are not identical amongst all sequence reads of a read family due to errors resulting from amplification and/or sequencing error. The sequencing reads of a read family may exhibit overlap when compared, for example by alignment. In some cases, the sequencing reads of a read family exhibit at least 75% identity, when optimally aligned. The term “percent (%) identity” refers to the percentage of identical residues shared between two sequences, e.g., a candidate sequence and a reference sequence, after aligning the sequences and introducing gaps, if necessary, to achieve the maximum percent identity (i.e., gaps can be introduced in one or both of the candidate and reference sequences for optimal alignment and, in some cases, non-homologous sequences can be disregarded for comparison purposes). Alignment, for purposes of determining percent identity, can be achieved in various ways, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence. Two sequencing reads of a family can exhibit at least 75% identity (e.g., at least 80%, 85%, 90%, or 95% identity) over any suitable length of bases, when optimally aligned. A first pair of sequencing reads in a read family can exhibit a % identity that is different from a second pair of sequencing reads. In some cases, the % identity is determined for an alignment over a length of at least 50 bases (e.g., at least 60 bases, 70 bases, 80 bases, 90 bases, 100 bases, 110 bases, 120 bases, 130 bases, 140 bases, or 150 bases). In some cases, the alignment is over a length of between about 25-250 bases, between about 50-200 bases, between about 75-175 bases, or between about 100-150 bases. In some cases, the alignment is over the entire length of the test sequence or the comparison sequence. In some embodiments, two sequencing reads of a read family exhibit at least 75% identity (e.g., at least 80%, 85%, 90%, or 95% identity) over a length of at least 50 bases (e.g., at least 60 bases, 70 bases, 80 bases, 90 bases, 100 bases, 110 bases, 120 bases, 130 bases, 140 bases, or 150 bases) when optimally aligned.

Amplified polynucleotides comprising linear concatemers of a circular polynucleotide template can comprise multiples repeats or copies of the circular polynucleotide template sequence. Sheared polynucleotides produced from an amplified polynucleotide can have various copies of the circular polynucleotide template sequence. A sheared polynucleotide can have less than one copy of the repeat sequence, at least one copy of the repeat sequence, at least two copies of the repeat sequence, or at least three copies of the repeat sequence. The number of repeats in sheared polynucleotides can depend on the length of the repeat sequence. For example, for sheared fragments of approximately the same size, a concatemer having repeats of relatively shorter length can yield sheared fragments having more copies of the repeat sequence compared a concatemer having repeats of longer length.

A sequencing read of a sheared polynucleotide or amplification product thereof can in some cases comprise at least one copy of the repeat sequence. In some cases, the sequencing read comprises at least two copies of the repeat sequence (e.g., at least three copies, four copies, or five copies). The average number of copies of the repeat sequence from sequence reads of a read family can depend on the length of the polynucleotides of the nucleic acid sample.

Sequencing reads can be grouped into read families by first identifying the length and/or sequence of the repeated segment in the concatemer, which corresponds to the sequence of the circular polynucleotide template. In some cases, identifying the length and/or sequence of the repeated segment comprises alignment of reads to other reads or alignment to reference sequences. Next, the junction sequence can be identified, for example by alignment to a reference sequence. The sequences of the 5′ and 3′ ends of the polynucleotide and their relative distances (e.g., in bases) from the junction can be determined. Reads having the same junction sequence and shared sequences at the 5′ and 3′ ends can be grouped into a read family, representing the sequencing reads of amplification products originating from the same sheared polynucleotide.

A sequence difference observed in a read family can be called a true sequence difference as opposed to a result of amplification and/or sequencing error, in some cases, by confirming that the sequence difference occurs in a second read family having the same junction sequence but different sequences at respective 5′ and 3′ ends (e.g., at least two sheared polynucleotides). Two read families having the same junction sequence but different 5′ and/or 3′ ends can correspond to two sheared polynucleotides of the same linear concatemer. Observing the sequence difference in two read families corresponding to the two sheared polynucleotides of the same amplified polynucleotide can be one way to confirm that the sequence difference is truly present on other circular polynucleotide and not the result of amplification and/or sequencing error in one of the sheared polynucleotides.

In some cases, a sequence difference observed in sequence reads of a read family is considered a sequence difference if the sequence difference occurs in a majority of the sequencing reads of the read family. In some cases, the sequence difference observed in sequence reads of the read family is considered a sequence difference if the sequence difference occurs in at least 50% of sequencing reads of the read family (e.g., at least 60%, 70%, 80%, 90%, or 95% of sequencing reads). In some cases, the sequence difference observed in sequence reads of the read family is considered a sequence difference if the sequence difference occurs in 100% of sequencing reads of the read family. In some cases, a sequence difference detected in the sequencing reads is called as the sequence variant when the sequence difference occurs in a majority of the sequencing reads from a first sheared polynucleotide and a majority of sequencing reads from a second sheared polynucleotide. In some cases, a sequence difference detected in the sequencing reads is called as the sequence variant when the sequence difference occurs in at least 50% of the sequencing reads (e.g., at least 60%, 70%, 80%, 90%, or 95% of sequencing reads) from the first sheared polynucleotide and at least 50% of sequencing reads (e.g., at least 60%, 70%, 80%, 90%, of 95% or sequencing reads) from the second sheared polynucleotide. In some cases, a sequence difference detected in the sequencing reads is called as the sequence variant when the sequence difference occurs in 100% of the sequencing reads from the first sheared polynucleotide and 100% of sequencing reads from the second sheared polynucleotide.

By using two different sheared polynucleotides, that is two sheared polynucleotides having the same junction sequence but different shear ends, to confirm the presence of a sequence difference identified from sequencing reads in a sample, sequence variant detection can be improved. True sequence variants are expected to be found in at least two sheared polynucleotides originating from the same amplified polynucleotide whereas errors are expected to be found in less than two sheared polynucleotides. In some cases, the error rate of variant detection is reduced. In some embodiments, the error rate of variant detection is reduced by at least 5%, 10%, 15%, 20%, 25%, 30%, 35%, 40%, 45%, or 50%. In some cases, the sensitivity and/or specificity of variant detection is increased. In some embodiments, the sensitivity of variant detection is increased by at least 5%, 10%, 15%, 20%, 25%, 30%, 35%, 40%, 45%, or 50%. In some embodiments, the specificity of variant detection is increased by at least 5%, 10%, 15%, 20%, 25%, 30%, 35%, 40%, 45%, or 50%. In some cases, the false positive rate is decreased.

In some cases, calling the sequence different as the sequence variant occurs further when (i) the sequence difference occurs in at least two circular polynucleotides having different junctions; (ii) the sequence difference is identified on both strands of a double-stranded input molecule; and/or (iii) the sequence difference occurs in a consensus sequence for a concatemer formed by amplification comprising rolling circle amplification (RCA). In some cases, the reference sequence is a sequencing read. In some cases, the reference sequence is a consensus sequence formed by aligning the sequencing reads with one another.

In some cases, the sheared polynucleotides are subjected to sequencing without enrichment. However, if desired, enriching one or more target polynucleotides among the amplified polynucleotides and/or sheared polynucleotides can be performed in an enrichment step prior to sequencing. Exemplary enrichment steps may include the use of nucleic acids with sequence complementary to a target sequence.

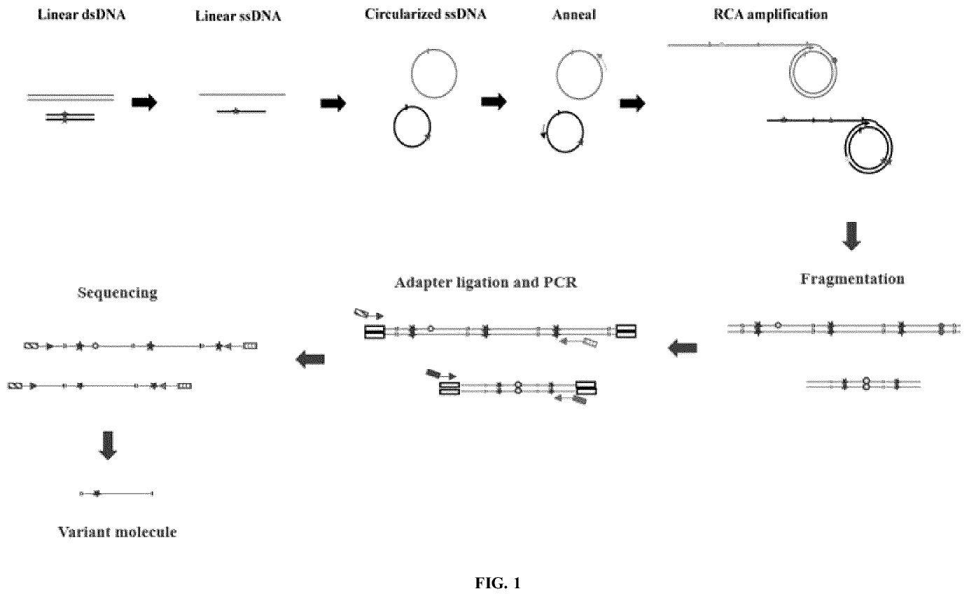

In one aspect, illustrated by FIG. 1 herein, a plurality of linear polynucleotides is obtained, such as linear double stranded polynucleotides, such as a linear double stranded DNA molecules. At least one of the plurality of double stranded polynucleotides has a sequence variant, marked by a star. The plurality of linear plurality double stranded polynucleotides is treated to obtain single stranded polynucleotides. Next, the linear single stranded polynucleotides are circularized and a primer is annealed to the circular polynucleotides. Circular polynucleotides are amplified from the annealed primer to create a concatemer comprising multiple copies of the starting polynucleotides. The concatemers are fragmented or sheared to create breaks in the concatemers and a second strand is added to the concatemers before or after fragmentation. Adapters are then ligated to the fragmented concatemers and the adapter-ligated concatemers are amplified using polymerase chain reaction (PCR) with primers binding to the adapters, or to a target sequence of the concatemer. In some cases, the primers comprise a barcode, an adapter, or a combination thereof. The PCR amplicons are then sequenced using any suitable method to identify sequence differences. The sequence differences are identified as the variant when the sequence difference is found in multiple copies of the polynucleotide of the concatemer and when it is found in concatemers having different breakpoints resulting from the fragmentation, thereby eliminating sequence differences resulting from errors, such as polymerase errors.

In another aspect, the disclosure provides a method of identifying a sequence variant in a nucleic acid sample comprising a plurality of polynucleotides, each polynucleotide of the plurality having a 5′ end and a 3′ end. In some cases, the method comprises (a) circularizing individual polynucleotides of the plurality to form a plurality of circular polynucleotides, wherein a given circular polynucleotide of the plurality has a junction sequence resulting from said circularization; (b) amplifying the circularized polynucleotides of (a) to produce a plurality of amplified polynucleotides; (c) shearing the amplified polynucleotides to produce sheared polynucleotides, each sheared polynucleotide comprising one or more shear points at a 5′ end and/or a 3′ end; (d) sequencing the sheared polynucleotides and/or amplification products of the sheared polynucleotides to produce a plurality of sequencing reads; and (d) calling a sequence difference detected in the sequencing reads as the sequence variant when the sequence difference occurs in sequencing reads corresponding to a first sheared polynucleotide and a second sheared polynucleotide.

In some cases, the method comprises (a) circularizing individual polynucleotides of said plurality to form a plurality of circular polynucleotides, each of which having a junction between the 5′ end and the 3′ end; (b) amplifying the circular polynucleotides of (a) to produce amplified polynucleotides; (c) shearing the amplified polynucleotides to produce sheared polynucleotides, each sheared polynucleotide comprising one or more shear points at a 5′ end and/or 3′ end; (d) sequencing the sheared polynucleotides to produce a plurality of sequencing reads; (e) identifying sequencing differences between sequencing reads and a reference sequence; and (f) calling a sequence difference as the sequence variant when the sequence difference occurs in at least two different sheared polynucleotides.

In general, joining ends of a polynucleotide to one-another to form a circular polynucleotide (either directly, or with one or more intermediate adapter oligonucleotides) produces a junction having a junction sequence. Where the 5′ end and 3′ end of a polynucleotide are joined via an adapter polynucleotide, the term “junction” can refer to a junction between the polynucleotide and the adapter (e.g. one of the 5′ end junction or the 3′ end junction), or to the junction between the 5′ end and the 3′ end of the polynucleotide as formed by and including the adapter polynucleotide. Where the 5′ end and the 3′ end of a polynucleotide are joined without an intervening adapter (e.g. the 5′ end and 3′ end of a single-stranded DNA), the term “junction” refers to the point at which these two ends are joined. A junction may be identified by the sequence of nucleotides comprising the junction (also referred to as the “junction sequence”).

In some embodiments, samples comprise polynucleotides having a mixture of ends formed by natural degradation processes (such as cell lysis, cell death, and other processes by which polynucleotides such as DNA and RNA are released from a cell to its surrounding environment in which it may be further degraded, e.g., cell-free polynucleotides, e.g., cell-free DNA and cell-free RNA), fragmentation that is a byproduct of sample processing (such as fixing, staining, and/or storage procedures), and fragmentation by methods that cleave DNA without restriction to specific target sequences (e.g. mechanical fragmentation, such as by sonication; non-sequence specific nuclease treatment, such as DNase I, fragmentase). Where samples comprise polynucleotides having a mixture of ends, the likelihood of two polynucleotides having the same 5′ end or 3′ end is low, and the likelihood that two polynucleotides will independently have both the same 5′ end and 3′ end is lower. Accordingly, in some embodiments, junctions may be used to distinguish different polynucleotides, even where the two polynucleotides comprise a portion having the same target sequence. Where polynucleotide ends are joined without an intervening adapter, a junction sequence may be identified by alignment to a reference sequence. For example, where the order of two component sequences appears to be reversed with respect to the reference sequence, the point at which the reversal appears to occur may be an indication of a junction at that point. Where polynucleotide ends are joined via one or more adapter sequences, a junction may be identified by proximity to the known adapter sequence, or by alignment as above if a sequencing read is of sufficient length to obtain sequence from both the 5′ and 3′ ends of the circularized polynucleotide. In some embodiments, the formation of a particular junction is a sufficiently rare event such that it is unique among the circularized polynucleotides of a sample.

In some embodiments, circularizing individual polynucleotides in (a) is effected by subjected the plurality of polynucleotides to a ligation reaction. The ligation reaction may comprise a ligase enzyme. In some embodiments, the ligase enzyme is degraded prior to amplifying in (b). Degradation of ligase prior to amplifying in (b) can increase the recovery rate of amplifiable polynucleotides. In some embodiments, the plurality of circularized polynucleotides are not purified or isolated prior to (b). In some embodiments, uncircularized, linear polynucleotides are degraded prior to amplifying.

In some cases, circularizing in (a) comprises the step of joining and adapter polynucleotide to the 5′ end, the 3′ end, or both the 5′ end and the 3′ end of a polynucleotide in the plurality of polynucleotides. As previously described, where the 5′ end and/or 3′ end of a polynucleotide are joined via an adapter polynucleotide, the term “junction” can refer to the junction between the polynucleotide and the adapter (e.g., one of the 5′ end junction or the 3′ end junction), or to the junction between the 5′ end and the 3′ end of the polynucleotide as formed by and including the adapter polynucleotide.

The circularized polynucleotides can be amplified, for example, after degradation of the ligase enzyme, to yield amplified polynucleotides. Amplifying the circular polynucleotides in (b) can be effected by a polymerase having strand-displacement activity. In some cases, the polymerase is a Phi29 DNA polymerase. In some cases, amplification comprises rolling circle amplification (RCA). The amplified polynucleotides resulting from RCA can comprise linear concatemers, or polynucleotides comprising two or more copies of a target sequence (e.g., subunit sequence) from a template polynucleotide. In some embodiments, amplifying comprises subjecting the circular polynucleotides to an amplification reaction mixture comprising random primers. In some cases, amplifying comprises subjecting the circular polynucleotides to an amplification reaction mixture comprising one or more primers, each of which specifically hybridizes to a different target sequence via sequence complementarity.

The amplified polynucleotides are sheared, in some cases, to produce sheared polynucleotides that are shorter in length relative to the unsheared polynucleotides. Two or more sheared polynucleotides originating from the same linear concatemer may have the same junction sequence but can have different 5′ and/or 3′ ends (e.g., shear ends).

Amplified polynucleotides can be sheared using any variety of methods, such as, but not limited to, physical fragmentation, enzymatic methods, and chemical fragmentation. Non-limiting examples of physical fragmentation methods that can be employed for the fragmentation of amplified polynucleotides include acoustic shearing, sonication, and hydrodynamic shearing. In some cases, acoustic shearing and sonication may be preferred. Non-limiting examples of enzymatic fragmentation methods that can be employed for the fragmentation of amplified polynucleotides include use of enzymes such as DNase I and other restriction endonucleases, including non-specific nucleases, and transposases. Non-limiting examples of chemical fragmentation methods that can be employed for the fragmentation of amplified polynucleotides include use of heat and divalent metal cations.

Sheared polynucleotides (also referred to as fragmented polynucleotides) which are shorter in length compared to the unsheared polynucleotides may be desired to match the capabilities of the sequencing instrument used for producing sequencing reads, also referred to as sequence reads. For example, amplified polynucleotides may be fragmented, for example sheared, to the optimal length determined by the downstream sequencing platform. Various sequencing instruments, further described herein, can accommodate nucleic acids of different lengths. In some cases, amplified polynucleotides are sheared in the process of attaching adapters useful in downstream sequencing platforms, for example in flow cell attachment or sequencing primer binding. In some cases, sheared polynucleotides are subject to amplification to produce amplification products of the sheared polynucleotides prior to sequencing. Additional amplification can be desirable, for example, to generate a sufficient amount of polynucleotides for downstream analysis, for example, sequencing analysis. The resulting amplification products can comprise multiple copies of individual sheared polynucleotides.

During sequencing, sheared polynucleotides or amplification products thereof originating from the same amplified polynucleotide can be sequenced. Sequencing reads resulting from sequencing can be grouped into read families. A read family can comprise any suitable number of sequence reads. In some cases, a read family comprises at least 5, 10, 15, 20, 25, 50, 75, or 100 sequences reads. In some cases, a group of sequence reads may not be identified as a read family unless a minimum number of sequence reads are present. For example, a read family can comprise at least 2, 3, 4, 5, 7, 8, 9, or 10 sequence reads. In some cases, a read family comprises at least 25 read sequences. In some cases, sequence reads which may be classified as a read family based on a shared junction sequence and shared sequences of the 5′ and 3′ ends. In some embodiments, the sequence reads of a read family have the same junction sequence. In some embodiments, the sequence reads of a read family have the same sequences at the 5′ and 3′ end, for example, the sequences may be identical over at least 5 bases, 6 bases, 7 bases, 8 bases, 9 bases, or 10 bases at each of the 5′ and 3′ ends. In some cases, the sequences at the 5′ and 3′ ends are not identical amongst all sequence reads of a read family due to errors resulting from amplification and/or sequencing error. The sequencing reads of a read family may exhibit overlap when compared, for example by alignment. In some cases, the sequencing reads of a read family exhibit at least 75% identity, when optimally aligned. The term “percent (%) identity” refers to the percentage of identical residues shared between two sequences, e.g., a candidate sequence and a reference sequence, after aligning the sequences and introducing gaps, if necessary, to achieve the maximum percent identity (i.e., gaps can be introduced in one or both of the candidate and reference sequences for optimal alignment and, in some cases, non-homologous sequences can be disregarded for comparison purposes). Alignment, for purposes of determining percent identity, can be achieved in various ways, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence. Two sequencing reads of a family can exhibit at least 75% identity (e.g., at least 80%, 85%, 90%, or 95% identity) over any suitable length of bases, when optimally aligned. A first pair of sequencing reads in a read family can exhibit a % identity that is different from a second pair of sequencing reads. In some cases, the % identity is determined for an alignment over a length of at least 50 bases (e.g., at least 60 bases, 70 bases, 80 bases, 90 bases, 100 bases, 110 bases, 120 bases, 130 bases, 140 bases, or 150 bases). In some cases, the alignment is over a length of between about 25-250 bases, between about 50-200 bases, between about 75-175 bases, or between about 100-150 bases. In some cases, the alignment is over the entire length of the test sequence or the comparison sequence. In some embodiments, two sequencing reads of a read family exhibit at least 75% identity (e.g., at least 80%, 85%, 90%, or 95% identity) over a length of at least 50 bases (e.g., at least 60 bases, 70 bases, 80 bases, 90 bases, 100 bases, 110 bases, 120 bases, 130 bases, 140 bases, or 150 bases) when optimally aligned.

Amplified polynucleotides comprising linear concatemers of a circular polynucleotide template can comprise multiples repeats or copies of the circular polynucleotide template sequence. Sheared polynucleotides produced from an amplified polynucleotide can have various copies of the circular polynucleotide template sequence. A sheared polynucleotide can have less than one copy of the repeat sequence, at least one copy of the repeat sequence, at least two copies of the repeat sequence, or at least three copies of the repeat sequence. The number of repeats in sheared polynucleotides can depend on the length of the repeat sequence. For example, for sheared fragments of approximately the same size, a concatemer having repeats of relatively shorter length can yield sheared fragments having more copies of the repeat sequence compared a concatemer having repeats of longer length.

A sequencing read of a sheared polynucleotide or amplification product thereof can in some cases comprise at least one copy of the repeat sequence. In some cases, the sequencing read comprises at least two copies of the repeat sequence (e.g., at least three copies, four copies, or five copies). The average number of copies of the repeat sequence from sequence reads of a read family can depend on the length of the polynucleotides of the nucleic acid sample.

Sequencing reads can be grouped into read families by first identifying the length and/or sequence of the repeated segment in the concatemer, which corresponds to the sequence of the circular polynucleotide template. In some cases, identifying the length and/or sequence of the repeated segment comprises alignment of reads to other reads or alignment to reference sequences. Next, the junction sequence can be identified, for example by alignment to a reference sequence. The sequences of the 5′ and 3′ ends of the polynucleotide and their relative distances (e.g., in bases) from the junction can be determined. Reads having the same junction sequence and shared sequences at the 5′ and 3′ ends can be grouped into a read family, representing the sequencing reads of amplification products originating from the same sheared polynucleotide.

A sequence difference observed in a read family can be called a true sequence difference as opposed to a result of amplification and/or sequencing error, in some cases, by confirming that the sequence difference occurs in a second read family having the same junction sequence but different sequences at respective 5′ and 3′ ends (e.g., at least two sheared polynucleotides). Two read families having the same junction sequence but different 5′ and/or 3′ ends can correspond to two sheared polynucleotides of the same linear concatemer. Observing the sequence difference in two read families corresponding to the two sheared polynucleotides of the same amplified polynucleotide can be one way to confirm that the sequence difference is truly present on other circular polynucleotide and not the result of amplification and/or sequencing error in one of the sheared polynucleotides.

In some cases, a sequence difference observed in sequence reads of a read family is considered a sequence difference if the sequence difference occurs in a majority of the sequencing reads of the read family. In some cases, the sequence difference observed in sequence reads of the read family is considered a sequence difference if the sequence difference occurs in at least 50% of sequencing reads of the read family (e.g., at least 60%, 70%, 80%, 90%, or 95% of sequencing reads). In some cases, the sequence difference observed in sequence reads of the read family is considered a sequence difference if the sequence difference occurs in 100% of sequencing reads of the read family. In some cases, a sequence difference detected in the sequencing reads is called as the sequence variant when the sequence difference occurs in a majority of the sequencing reads from a first sheared polynucleotide and a majority of sequencing reads from a second sheared polynucleotide. In some cases, a sequence difference detected in the sequencing reads is called as the sequence variant when the sequence difference occurs in at least 50% of the sequencing reads (e.g., at least 60%, 70%, 80%, 90%, or 95% of sequencing reads) from the first sheared polynucleotide and at least 50% of sequencing reads (e.g., at least 60%, 70%, 80%, 90%, of 95% or sequencing reads) from the second sheared polynucleotide. In some cases, a sequence difference detected in the sequencing reads is called as the sequence variant when the sequence difference occurs in 100% of the sequencing reads from the first sheared polynucleotide and 100% of sequencing reads from the second sheared polynucleotide.

By using two different sheared polynucleotides, that is two sheared polynucleotides having the same junction sequence but different shear ends, to confirm the presence of a sequence difference identified from sequencing reads in a sample, sequence variant detection can be improved. True sequence variants are expected to be found in at least two sheared polynucleotides originating from the same amplified polynucleotide whereas errors are expected to be found in less than two sheared polynucleotides. In some cases, the error rate of variant detection is reduced. In some embodiments, the error rate of variant detection is reduced by at least 5%, 10%, 15%, 20%, 25%, 30%, 35%, 40%, 45%, or 50%. In some cases, the sensitivity and/or specificity of variant detection is increased. In some embodiments, the sensitivity of variant detection is increased by at least 5%, 10%, 15%, 20%, 25%, 30%, 35%, 40%, 45%, or 50%. In some embodiments, the specificity of variant detection is increased by at least 5%, 10%, 15%, 20%, 25%, 30%, 35%, 40%, 45%, or 50%. In some cases, the false positive rate is decreased.

In some cases, calling the sequence different as the sequence variant occurs further when (i) the sequence difference occurs in at least two circular polynucleotides having different junctions; (ii) the sequence difference is identified on both strands of a double-stranded input molecule; and/or (iii) the sequence difference occurs in a consensus sequence for a concatemer formed by amplification comprising rolling circle amplification (RCA). In some cases, the reference sequence is a sequencing read. In some cases, the reference sequence is a consensus sequence formed by aligning the sequencing reads with one another.

In some cases, the sheared polynucleotides are subjected to sequencing without enrichment. However, if desired, enriching one or more target polynucleotides among the amplified polynucleotides and/or sheared polynucleotides can be performed in an enrichment step prior to sequencing. Exemplary enrichment steps may include the use of nucleic acids with sequence complementary to a target sequence.

The sequence variant, as described further herein, can be any variation with respect to the reference sequence. Non-limiting examples of sequence variants that can be detected using methods herein include single nucleotide polymorphisms (SNP), deletion/insertion polymorphisms (DIP), copy number variants (CNV), short tandem repeats (STR), simple sequence repeats (SSR), variable number of tandem repeats (VNTR), amplified fragment length polymorphisms (AFLP), retrotransposon-based insertion polymorphisms, sequence specific amplified polymorphism, and differences in epigenetic marks that can be detected as sequence variants (e.g. methylation differences). In some cases, the sequence variant is a polymorphism, such as a single-nucleotide polymorphism. In some cases, the sequence variant is a causal genetic variant. In some cases, the sequence variant is associated with a type or stage of cancer.

The nucleic acid sample can be a sample from a subject. In some cases, the sample is from a human subject. In some cases, the sample comprises urine, stool, blood, saliva, tissue, or bodily fluid from a subject, such as a human subject. In some cases, the sample comprises tumor cells. In some cases, the sample comprises a formalin-fixed paraffin embedded sample. In some cases, the plurality of polynucleotides of the sample comprises cell-free polynucleotides. The cell-free polynucleotides may comprise cell-free DNA, and in some cases, circulating tumor DNA and/or circulating tumor RNA. The cell-free polynucleotides may comprise cell-free RNA. In some embodiments, the method further comprises diagnosing, and optionally treating, the subject based on calling of the sequence variant. In some cases, a microbial contaminant in a sample is identified based on calling of the sequence variant. In such cases, the sample can be from a subject but may also be from a non-subject sample such as a soil sample or food sample.

The plurality of polynucleotides can be single-stranded. In some cases, the polynucleotides are in double-stranded form and are treated, for example by denaturation, to yield single-strands before proceeding with the circularization. In some cases, double-stranded polynucleotides are circularized to yield double-stranded circles and the double-stranded circles are treated, for example by denaturation, to yield single-stranded circles.

In another aspect, the disclosure provides a method of identifying a sequence variant in a nucleic acid sample comprising a plurality of polynucleotides, each polynucleotide of the plurality having a 5′ end and a 3′ end. In some embodiments, the method comprises: (a) circularizing individual polynucleotides of the plurality to form a plurality of circular polynucleotides, wherein a given circular polynucleotide has a junction sequence resulting from said circularization; (b) amplifying the circular polynucleotides of (a) to produce a plurality of amplified polynucleotides, wherein a first amplified polynucleotide of the plurality and a second amplified polynucleotide of the plurality comprise the junction sequence but comprise different sequences at their respective 5′ and/or 3′ ends; (c) sequencing the plurality of amplified polynucleotides and/or amplification products thereof to produce a plurality of sequencing reads corresponding to the first amplified polynucleotide and the second amplified polynucleotide; and (d) calling a sequence difference detected in the sequencing reads as the sequence variant when the sequence difference occurs in sequencing reads corresponding to both the first amplified polynucleotide and the second amplified polynucleotide. In some embodiments, circularizing individual polynucleotides in (a) is effected by a ligase enzyme. In some embodiments, the ligase enzyme is degraded prior to amplifying in (b). Degradation of ligase prior to amplifying in (b) can increase the recovery rate of amplifiable polynucleotides. In some embodiments, the plurality of circularized polynucleotides is not purified or isolated prior to (b).

In some cases, circularizing in (a) comprises the step of joining an adapter polynucleotide to the 5′ end, the 3′ end, or both the 5′ end and the 3′ end of a polynucleotide in the plurality of polynucleotides. As previously described, where the 5′ end and/or 3′ end of a polynucleotide are joined via an adapter polynucleotide, the term “junction” can refer to the junction between the polynucleotide and the adapter (e.g., one of the 5′ end junction or the 3′ end junction), or to the junction between the 5′ end and the 3′ end of the polynucleotide as formed by and including the adapter polynucleotide.

Following circularization, the circular polynucleotides are amplified. Amplifying the circular polynucleotides in (b) can be effected by a polymerase having strand-displacement activity. In some cases, the polymerase is a Phi29 DNA polymerase. In some cases, amplifying the circular polynucleotides in (b) comprises rolling circle amplification (RCA). Rolling circle amplification can result in amplification polynucleotides comprising linear concatemers of the template circular polynucleotide sequence. In some cases, amplifying in (b) comprises subjecting the circular polynucleotides to an amplification reaction mixture using random primers. Random primers which can non-specifically (e.g., randomly) hybridize to the circular polynucleotides during the amplifying of (b). Random primers which can non-specifically hybridize to circular polynucleotides can hybridize to a common circular polynucleotide, a plurality of circular polynucleotides, or both. In some cases, two or more random primers hybridize to the same circular polynucleotide (e.g., different regions of the same circular polynucleotide) and yield amplified polynucleotides having repeats of the same target sequence (or subunit sequence). Amplified polynucleotides of the same template (e.g., circular polynucleotide) can have the same junction sequence. In some embodiments, individual random primers comprise sequences at their respective 5′ and/or 3′ ends distinct from each other, and the resulting amplified polynucleotides can have sequences at 5′ and/or 3′ ends distinct from each other. Amplified polynucleotides of the same template, in some cases, have different 5′ and/or 3′ ends, depending on where the primer initially bound and where nucleotide incorporation was terminated. In some cases, amplifying in (b) comprises subjecting the circular polynucleotides to an amplification reaction mixture comprising target specific primers. Target specific primers can refer to primers targeting particular gene sequences, or in some cases refers to primers targeting adapter polynucleotide sequences. Amplified polynucleotides resulting from the use of target specific primers can share a common first end (e.g., primer) and may not share a second end, depending on where nucleotide incorporation was terminated. Amplifying can comprise multiple cycles of denaturation, primer binding, and primer extension. In some cases, the amplified polynucleotides can be subjected to further amplification to yield amplification products of the amplified polynucleotides. Additional amplification can be desirable, for example, to generate a sufficient amount of polynucleotides for downstream analysis, for example, sequencing analysis. The resulting amplification products can comprise multiple copies of individual amplified polynucleotides.

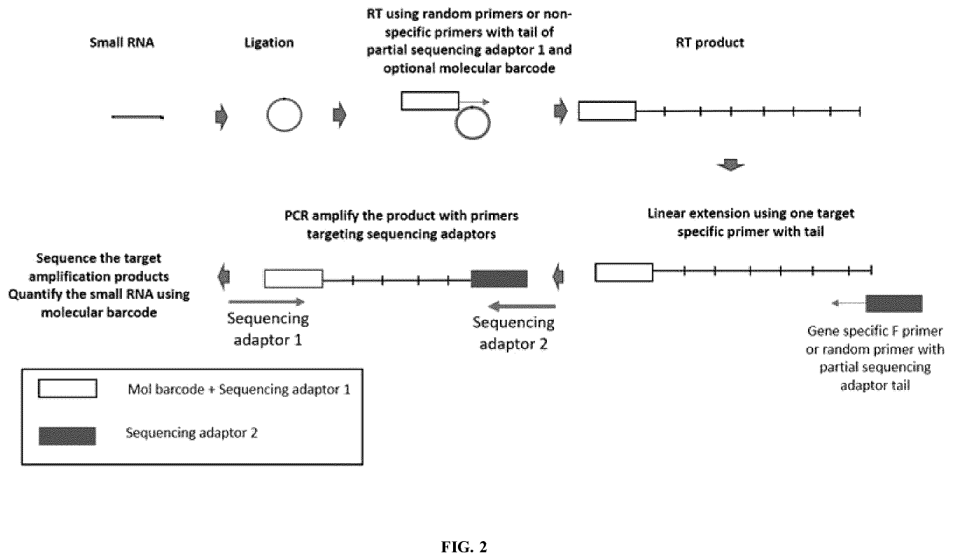

In one aspect, illustrated by FIG. 2, a small RNA is amplified for various applications, such as but not limited to sequencing and quantification. The small RNA is circularized using ligation and the circularized RNA is amplified using reverse transcriptase and primers having a sequence adapter and/or a molecular barcode. The resulting reverse transcriptase product is amplified using linear extension of a target specific primer with a sequencing adapter. This product is amplified using PCR and primers that bind to each of the adapters. The amplification products are sequenced. The small RNA can be quantified from the sequence information using the molecular barcode.

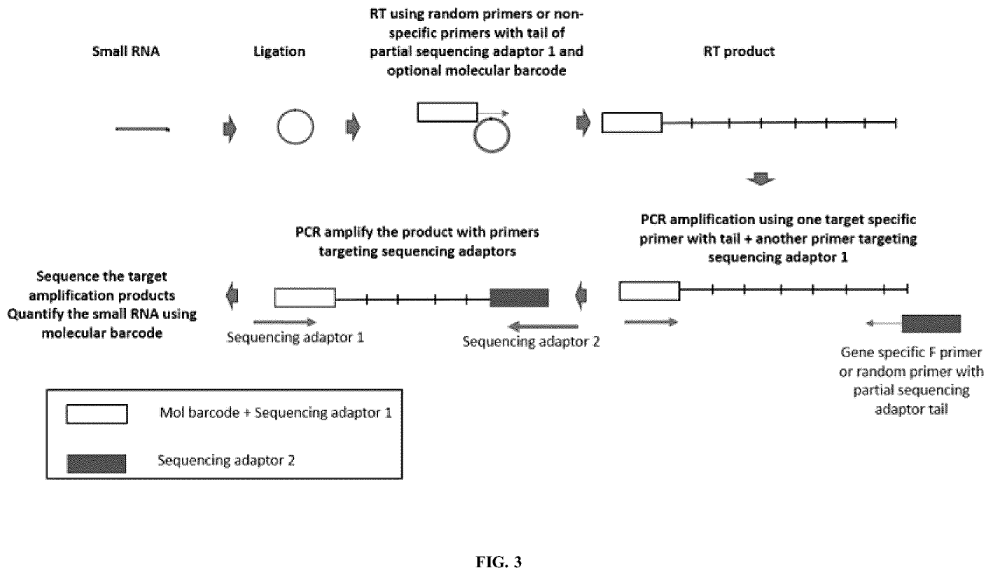

In another aspect, illustrated by FIG. 3, a small RNA is amplified for various applications, such as but not limited to sequencing and quantification. The small RNA is circularized using ligation and the circularized RNA is amplified using reverse transcriptase and primers having a sequence adapter and/or a molecular barcode. The resulting reverse transcriptase product is amplified using PCR with primers annealing to the sequencing adapter and a primer that anneals to the reverse transcriptase product that also has an adapter sequence. The PCR product is further amplified using primers that bind to each adapter sequence. The final amplification product is sequenced. The small RNA can be quantified from the sequence information using the molecular barcode.

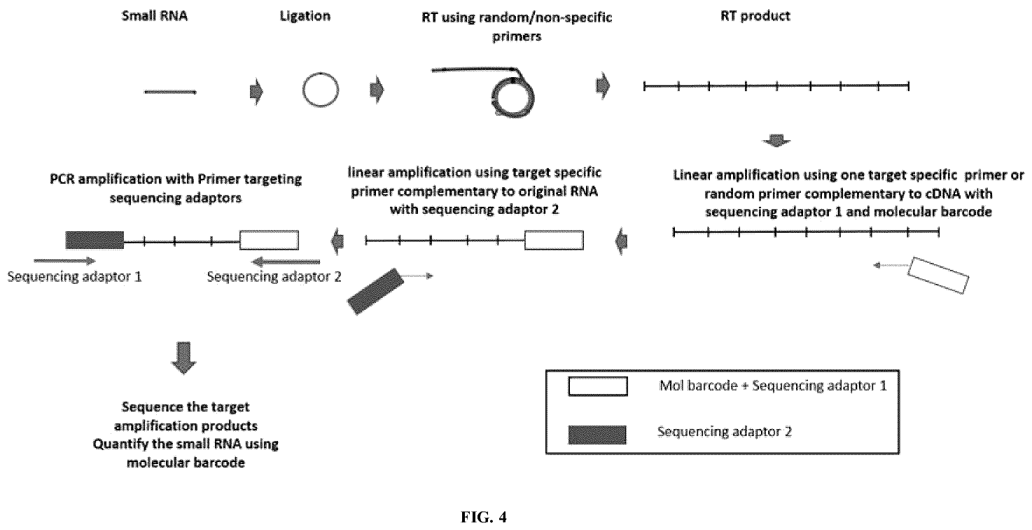

In another aspect, illustrated by FIG. 4, a small RNA is amplified for various applications such as but not limited to sequencing and quantification. The small RNA is circularized using ligation and the circularized RNA is amplified using reverse transcriptase and random primers. The reverse transcriptase product is subjected to linear amplification using a target specific primer or a random primer with a sequencing adapter and/or a molecular barcode. The linear amplification product is subjected to further linear amplification using a target specific primer with a second sequencing adapter. This product is subjected to PCR using primers that bind to each of the sequencing adapters. The PCR products are sequenced and the small RNAs can be quantified using the molecular barcode.

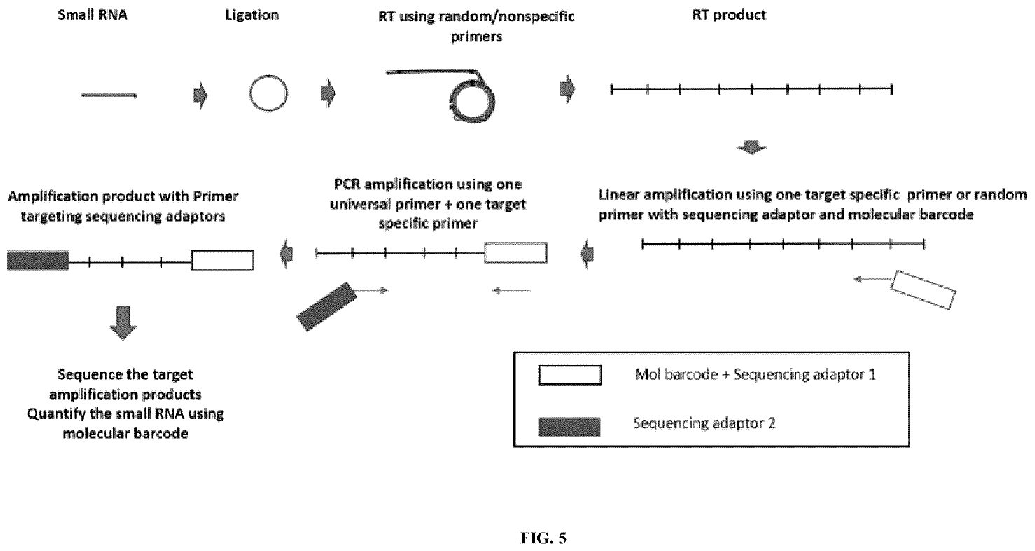

In another aspect, illustrated by FIG. 5, a small RNA is amplified for various applications such as but not limited to sequencing and quantification. The small RNA is circularized using ligation and the circularized RNA is amplified using reverse transcriptase and random primers. The reverse transcriptase product is subjected to linear amplification using a target specific primer or a random primer with a sequencing adapter and/or a molecular barcode. The linear amplification product is subjected to PCR amplification using a target specific primer having an adapter and a primer that binds to the adapter. The PCR product is subjected to further amplification using primers that bind to the adapters and the amplification products are sequenced. The small RNA can be quantified using the molecular barcode.

The amplified polynucleotides and/or amplification products thereof can be subsequently sequenced to yield sequencing reads. In some cases, the amplified polynucleotides and/or amplification products are subjected to sequencing without enrichment. However, if desired, enriching one or more target polynucleotides among the amplified polynucleotides and/or amplification products can be performed in an enrichment step prior to sequencing.

Sequencing reads can be grouped into read families. A read family can comprise any suitable number of sequence reads. In some cases, a read family comprises at least 5, 10, 15, 20, 25, 50, 75, or 100 sequence reads. In some cases, a group of sequence reads may not be identified as a read family unless a minimum number of sequence reads are present. For example, a read family comprises at least 2, 3, 4, 5, 6, 7, 8, 9, or 10 sequence reads. In some cases, a read family comprises at least 25 read sequences. In some embodiments, the sequence reads of a read family have the same junction sequence. In some embodiments, the sequence reads of a read family have the same sequences at the 5′ and 3′ ends, for example, the sequences may be identical over at least 5 bases, 6 bases, 7 bases, 8 bases, 9 bases, or 10 bases at each of the 5′ and 3′ ends. In some cases, the sequences at the 5′ and 3′ ends are not identical amongst all sequence reads of a read family due to errors resulting from amplification and/or sequencing. The sequencing reads of a read family may exhibit overlap when compared, for example by alignment. In some cases, the sequencing reads of a read family exhibit at least 75% identity, when optimally aligned. Two sequencing reads of a family can exhibit at least 75% identity (e.g., at least 80%, 85%, 90%, or 95% identity) over any suitable length of bases, when optimally aligned. A first pair of sequencing reads in a read family can exhibit a % identity that is different from a second pair of sequencing reads in the read family. In some cases, the % identity is determined for an alignment over a length of at least 50 bases (e.g., at least 60 bases, 70 bases, 80 bases, 90 bases, 100 bases, 110 bases, 120 bases, 130 bases, 140 bases, or 150 bases). In some cases, the alignment is over a length of between about 25-250 bases, between about 50-200 bases, between about 75-175 bases, or between about 100-150 bases. In some cases, the alignment is over the entire length of the test sequence or the comparison sequence. In some embodiments, two sequencing reads of a read family exhibit at least 75% identity (e.g., at least 80%, 85%, 90%, or 95% identity) over a length of at least 50 bases (e.g., at least 60 bases, 70 bases, 80 bases, 90 bases, 100 bases, 110 bases, 120 bases, 130 bases, 140 bases, or 150 bases) when optimally aligned.

Amplified polynucleotides comprising linear concatemers of a shared circular polynucleotide template can yield multiple linear concatemers of the same circular polynucleotide sequence but on multiple, individual molecules. A sequencing read of an amplified polynucleotide or amplification product thereof can in some cases comprise at least one copy of the repeat sequence. In some cases, the sequencing read comprises at least two copies of the repeat sequence (e.g., at least three copies, four copies, or five copies). The average number of copies of the repeat sequence from sequence reads of a read family can depend on the length of the polynucleotides of the nucleic acid sample. For example, a sample comprising relatively longer polynucleotides may result in concatemers with fewer repeats compared to a sample comprising relatively shorter polynucleotides if the concatemers are similar in length.

Sequencing reads can be grouped into read families by first identifying the length and/or sequence of the repeated segment in the concatemer, which corresponds to the sequence of the circular polynucleotide template. In some cases, identifying the length and/or sequence of the repeated segment comprises alignment of reads to other reads or alignment to reference sequences. Next, the junction sequence can be identified, for example by alignment to a reference sequence. The sequences of the 5′ and 3′ ends of the polynucleotide and their relative distances (e.g., in bases) from the junction can be determined. Reads having the same junction sequence and shared sequences at the 5′ and 3′ ends can be grouped into a read family, representing the sequencing reads of amplification products originating from the same amplified polynucleotide, or the same molecular copy of the circular polynucleotide.

A sequence difference observed in a read family can be called a true sequence difference as opposed to a result of amplification and/or sequencing error, in some cases, by confirming that the sequence difference occurs in a second read family having the same junction sequence but different sequences at respective 5′ and 3′ ends. Two read families having the same junction sequence but different 5′ and/or 3′ ends can correspond to two amplified polynucleotides of the same circular polynucleotide. Observing the sequence difference in two read families corresponding to the same circular polynucleotide can be one way to confirm that the sequence difference is truly present on the circular polynucleotide and not the result of amplification and/or sequencing error in one of the amplified polynucleotides.

In some cases, a sequence difference observed in sequence reads of a read family is considered a sequence difference if the sequence difference occurs in a majority of the sequencing reads of the read family. In some cases, the sequence difference observed in sequence reads of the read family is considered a sequence difference if the sequence difference occurs in at least 50% of sequencing reads of the read family (e.g., at least 60%, 70%, 80%, 90%, or 95% of sequencing reads). In some cases, the sequence difference observed in sequence reads of the read family is considered a sequence difference if the sequence difference occurs in 100% of sequencing reads of the read family. In some cases, a sequence difference detected in the sequencing reads is called as the sequence variant when the sequence difference occurs in a majority of the sequencing reads from a first amplified polynucleotide and a majority of sequencing reads from a second amplified polynucleotide. In some cases, a sequence difference detected in the sequencing reads is called as the sequence variant when the sequence difference occurs in at least 50% of the sequencing reads (e.g., at least 60%, 70%, 80%, 90%, or 95% of sequencing reads) from the first amplified polynucleotide and at least 50% of sequencing reads (e.g., at least 60%, 70%, 80%, 90%, or 95% of sequencing reads) from the second amplified polynucleotide. In some cases, a sequence difference detected in the sequencing reads is called as the sequence variant when the sequence difference occurs in 100% of the sequencing reads from the first amplified polynucleotide and 100% of sequencing reads from the second amplified polynucleotide.

In practicing the methods described herein, variant detection in a sample comprising a plurality of polynucleotides can be improved. In some cases, the error rate of variant detection is reduced. In some embodiments, the error rate of variant detection is reduced by at least 5%, 10%, 15%, 20%, 25%, 30%, 35%, 40%, 45%, or 50%. In some cases, the sensitivity and/or specificity of variant detection is increased. In some embodiments, the sensitivity of variant detection is increased by at least 5%, 10%, 15%, 20%, 25%, 30%, 35%, 40%, 45%, or 50%. In some embodiments, the specificity of variant detection is increased by at least 5%, 10%, 15%, 20%, 25%, 30%, 35%, 40%, 45%, or 50%. In some cases, the false positive rate is decreased.

The sequence variant, as described further herein, can be any variation with respect to the reference sequence. Non-limiting examples of sequence variants that can be detected using methods herein include single nucleotide polymorphisms (SNP), deletion/insertion polymorphisms (DIP), copy number variants (CNV), short tandem repeats (STR), simple sequence repeats (SSR), variable number of tandem repeats (VNTR), amplified fragment length polymorphisms (AFLP), retrotransposon-based insertion polymorphisms, sequence specific amplified polymorphism, and differences in epigenetic marks that can be detected as sequence variants (e.g. methylation differences). In some cases, the sequence variant is a polymorphism, such as a single-nucleotide polymorphism.

The nucleic acid sample can be a sample from a subject. In some cases, the sample is from a human subject. In some cases, the sample comprises urine, stool, blood, saliva, tissue, or bodily fluid from a subject, such as a human subject. In some cases, the sample comprises tumor cells. In some cases, the sample comprises a formalin-fixed paraffin embedded sample. In some cases, the plurality of polynucleotides of the sample comprises cell-free polynucleotides. The cell-free polynucleotides may comprise cell-free DNA, and in some cases, circulating tumor DNA. The cell-free polynucleotides may comprise cell-free RNA, and in some cases, circulating tumor RNA.

As previously described, the plurality of polynucleotides can be single-stranded. In some cases, the polynucleotides are in double-stranded form and are treated, for example by denaturation, to yield single-strands before proceeding with the circularization. In some cases, double-stranded polynucleotides are circularized to yield double-stranded circles and the double-stranded circles are treated, for example by denaturation, to yield single-stranded circles.

In another aspect, the disclosure provides a method of performing rolling circle amplification, such as in a nucleic acid sample comprising a plurality of polynucleotides. In some embodiments, each polynucleotide of the plurality has a 5′ end and a 3′ end, and the method comprises: (a) circularizing individual polynucleotides of the plurality to form a plurality of circular polynucleotides using a ligase enzyme, each polynucleotide having a junction between the 5′ end and 3′ end; (b) degrading the ligase enzyme; and (c) amplifying the circular polynucleotides of (a) after degrading the ligase enzyme, wherein polynucleotides are not purified or isolated between steps (a) and (c). In some embodiments, the method comprises additional steps of (d) sequencing the amplified polynucleotides to produce a plurality of sequencing reads; (e) identifying sequence differences between sequencing reads and a reference sequence; and (f) calling a sequence difference that occurs in at least two circular polynucleotides having different junctions as the sequence variant. In some embodiments, the method comprises identifying sequence differences between sequencing reads and a reference sequence, and calling a sequence difference that occurs in at least two circular polynucleotides having different junctions as the sequence variant, wherein: (a) the sequencing reads correspond to amplification products of the at least two circular polynucleotides; and (b) each of the at least two circular polynucleotides comprises a different junction formed by ligating a 5′ end and 3′ end of the respective polynucleotides.

In another aspect, the disclosure provides a method of performing rolling circle amplification, such as in a nucleic acid sample comprising a plurality of polynucleotides. In some embodiments, each polynucleotide of the plurality has a 5′ end and a 3′ end, and the method comprises: (a) circularizing individual polynucleotides of the plurality using a ligase enzyme to form a plurality of circular polynucleotides, each polynucleotide having a junction between the 5′ end and 3′ end; (b) degrading the ligase enzyme; (c) amplifying the circular polynucleotides of (a) after degrading the ligase enzyme to produce amplified polynucleotides, wherein polynucleotides are not purified or isolated between steps (a) and (c); (d) shearing the amplified polynucleotides to produce sheared polynucleotides, each sheared polynucleotide comprising one or more shear points at a 5′ end and/or a 3′ end. In some embodiments, the method comprises additional steps of (e) sequencing the sheared polynucleotides to produce a plurality of sequencing reads; (f) identifying sequence differences between sequencing reads and a reference sequence; and (g) calling a sequence difference as the sequence variant when the sequence difference occurs in at least two different sheared polynucleotides. Degradation of ligase prior to amplifying in (c) can increase the recovery rate of amplifiable polynucleotides.

In some embodiments, the method comprises identifying sequence differences between sequencing reads and a reference sequence, and calling a sequence difference that occurs in at least two circular polynucleotides having different junctions as the sequence variant, wherein: (a) the sequencing reads correspond to amplification products of the at least two circular polynucleotides; and (b) each of the at least two circular polynucleotides comprises a different junction formed by ligating a 5′ end and 3′ end of the respective polynucleotides. In some embodiments, the method comprises calling the sequence difference as the sequence variant occurs further when (i) the sequence difference occurs in at least two circular polynucleotides having different junctions; (ii) the sequence difference is identified on both strands of a double-stranded input molecule; and/or (iii) the sequence difference occurs in a consensus sequence for a concatemer formed by amplification comprising rolling circle amplification.

In general, the term “sequence variant” refers to any variation in sequence relative to one or more reference sequences. Typically, the sequence variant occurs with a lower frequency than the reference sequence for a given population of individuals for whom the reference sequence is known. For example, a particular bacterial genus may have a consensus reference sequence for the 16S rRNA gene, but individual species within that genus may have one or more sequence variants within the gene (or a portion thereof) that are useful in identifying that species in a population of bacteria. As a further example, sequences for multiple individuals of the same species (or multiple sequencing reads for the same individual) may produce a consensus sequence when optimally aligned, and sequence variants with respect to that consensus may be used to identify mutants in the population indicative of dangerous contamination. In general, a “consensus sequence” refers to a nucleotide sequence that reflects the most common choice of base at each position in the sequence where the series of related nucleic acids has been subjected to intensive mathematical and/or sequence analysis, such as optimal sequence alignment according to any of a variety of sequence alignment algorithms. A variety of alignment algorithms are available, some of which are described herein. In some embodiments, the reference sequence is a single known reference sequence, such as the genomic sequence of a single individual. In some embodiments, the reference sequence is a consensus sequence formed by aligning multiple known sequences, such as the genomic sequence of multiple individuals serving as a reference population, or multiple sequencing reads of polynucleotides from the same individual. In some embodiments, the reference sequence is a consensus sequence formed by optimally aligning the sequences from a sample under analysis, such that a sequence variant represents a variation relative to corresponding sequences in the same sample. In some embodiments, the sequence variant occurs with a low frequency in the population (also referred to as a “rare” sequence variant). For example, the sequence variant may occur with a frequency of about or less than about 5%, 4%, 3%, 2%, 1.5%, 1%, 0.75%, 0.5%, 0.25%, 0.1%, 0.075%, 0.05%, 0.04%, 0.03%, 0.02%, 0.01%, 0.005%, 0.001%, or lower. In some embodiments, the sequence variant occurs with a frequency of about or less than about 0.1%.

A sequence variant can be any variation with respect to a reference sequence. A sequence variation may consist of a change in, insertion of, or deletion of a single nucleotide, or of a plurality of nucleotides (e.g. 2, 3, 4, 5, 6, 7, 8, 9, 10, or more nucleotides). Where a sequence variant comprises two or more nucleotide differences, the nucleotides that are different may be contiguous with one another, or discontinuous. Non-limiting examples of types of sequence variants include single nucleotide polymorphisms (SNP), deletion/insertion polymorphisms (DIP), copy number variants (CNV), short tandem repeats (STR), simple sequence repeats (SSR), variable number of tandem repeats (VNTR), amplified fragment length polymorphisms (AFLP), retrotransposon-based insertion polymorphisms, sequence specific amplified polymorphism, and differences in epigenetic marks that can be detected as sequence variants (e.g. methylation differences).

Nucleic acid samples that may be subjected to methods described herein can be derived from any suitable source. In some embodiments, the samples used are environmental samples. Environmental sample may be from any environmental source, for example, naturally occurring or artificial atmosphere, water systems, soil, or any other sample of interest. In some embodiments, the environmental samples may be obtained from, for example, atmospheric pathogen collection systems, sub-surface sediments, groundwater, ancient water deep within the ground, plant root-soil interface of grassland, coastal water, and sewage treatment plants.

Polynucleotides from a sample may be any of a variety of polynucleotides, including but not limited to, DNA, RNA, ribosomal RNA (rRNA), transfer RNA (tRNA), micro RNA (miRNA), messenger RNA (mRNA), fragments of any of these, or combinations of any two or more of these. In some embodiments, samples comprise DNA. In some embodiments, samples comprise genomic DNA. In some embodiments, samples comprise mitochondrial DNA, chloroplast DNA, plasmid DNA, bacterial artificial chromosomes, yeast artificial chromosomes, oligonucleotide tags, or combinations thereof. In some embodiments, the samples comprise DNA generated by amplification, such as by primer extension reactions using any suitable combination of primers and a DNA polymerase, including but not limited to polymerase chain reaction (PCR), reverse transcription, and combinations thereof. Where the template for the primer extension reaction is RNA, the product of reverse transcription is referred to as complementary DNA (cDNA). Primers useful in primer extension reactions can comprise sequences specific to one or more targets, random sequences, partially random sequences, and combinations thereof. In general, sample polynucleotides comprise any polynucleotide present in a sample, which may or may not include target polynucleotides. The polynucleotides may be single-stranded, double-stranded, or a combination of these. In some embodiments, polynucleotides subjected to a method of the disclosure are single-stranded polynucleotides, which may or may not be in the presence of double-stranded polynucleotides. In some embodiments, the polynucleotides are single-stranded DNA. Single-stranded DNA (ssDNA) may be ssDNA that is isolated in a single-stranded form, or DNA that is isolated in double-stranded form and subsequently made single-stranded for the purpose of one or more steps in a method of the disclosure.

In some embodiments, polynucleotides are subjected to subsequent steps (e.g. circularization and amplification) without an extraction step, and/or without a purification step. For example, a fluid sample may be treated to remove cells without an extraction step to produce a purified liquid sample and a cell sample, followed by isolation of DNA from the purified fluid sample. A variety of procedures for isolation of polynucleotides are available, such as by precipitation or non-specific binding to a substrate followed by washing the substrate to release bound polynucleotides. Where polynucleotides are isolated from a sample without a cellular extraction step, polynucleotides will largely be extracellular or “cell-free” polynucleotides, such as cell-free DNA and cell-free RNA, which may correspond to dead or damaged cells. The identity of such cells may be used to characterize the cells or population of cells from which they are derived, such as tumor cells (e.g. in cancer detection), fetal cells (e.g. in prenatal diagnostic), cells from transplanted tissue (e.g. in early detection of transplant failure), or members of a microbial community.

If a sample is treated to extract polynucleotides, such as from cells in a sample, a variety of extraction methods are available. For example, nucleic acids can be purified by organic extraction with phenol, phenol/chloroform/isoamyl alcohol, or similar formulations, including TRIzol and TriReagent. Other non-limiting examples of extraction techniques include: (1) organic extraction followed by ethanol precipitation, e.g., using a phenol/chloroform organic reagent (Ausubel et al., 1993), with or without the use of an automated nucleic acid extractor, e.g., the Model 341 DNA Extractor available from Applied Biosystems (Foster City, Calif.); (2) stationary phase adsorption methods (U.S. Pat. No. 5,234,809; Walsh et al., 1991); and (3) salt-induced nucleic acid precipitation methods (Miller et al., (1988), such precipitation methods being typically referred to as “salting-out” methods. Another example of nucleic acid isolation and/or purification includes the use of magnetic particles to which nucleic acids can specifically or non-specifically bind, followed by isolation of the beads using a magnet, and washing and eluting the nucleic acids from the beads (see e.g. U.S. Pat. No. 5,705,628). In some embodiments, the above isolation methods may be preceded by an enzyme digestion step to help eliminate unwanted protein from the sample, e.g., digestion with proteinase K, or other like proteases. See, e.g., U.S. Pat. No. 7,001,724. If desired, RNase inhibitors may be added to the lysis buffer. For certain cell or sample types, it may be desirable to add a protein denaturation/digestion step to the protocol. Purification methods may be directed to isolate DNA, RNA, or both. When both DNA and RNA are isolated together during or subsequent to an extraction procedure, further steps may be employed to purify one or both separately from the other. Sub-fractions of extracted nucleic acids can also be generated, for example, purification by size, sequence, or other physical or chemical characteristic. In addition to an initial nucleic acid isolation step, purification of nucleic acids can be performed after any step in the disclosed methods, such as to remove excess or unwanted reagents, reactants, or products. A variety of methods for determining the amount and/or purity of nucleic acids in a sample are available, such as by absorbance (e.g. absorbance of light at 260 nm, 280 nm, and a ratio of these) and detection of a label (e.g. fluorescent dyes and intercalating agents, such as SYBR green, SYBR blue, DAPI, propidium iodine, Hoechst stain, SYBR gold, ethidium bromide).

Where desired, polynucleotides from a sample may be fragmented prior to further processing. Fragmentation may be accomplished by any of a variety of methods, including chemical, enzymatic, and mechanical fragmentation. In some embodiments, the fragments have an average or median length from about 10 to about 1,000 nucleotides in length, such as between 10-800, 10-500, 50-500, 90-200, or 50-150 nucleotides. In some embodiments, the fragments have an average or median length of about or less than about 100, 200, 300, 500, 600, 800, 1000, or 1500 nucleotides. In some embodiments, the fragments range from about 90-200 nucleotides, and/or have an average length of about 150 nucleotides. In some embodiments, the fragmentation is accomplished mechanically comprising subjecting sample polynucleotides to acoustic sonication. In some embodiments, the fragmentation comprises treating the sample polynucleotides with one or more enzymes under conditions suitable for the one or more enzymes to generate double-stranded nucleic acid breaks. Examples of enzymes useful in the generation of polynucleotide fragments include sequence specific and non-sequence specific nucleases. Non-limiting examples of nucleases include DNase I, Fragmentase, restriction endonucleases, variants thereof, and combinations thereof. For example, digestion with DNase I can induce random double-stranded breaks in DNA in the absence of Mg++ and in the presence of Mn++. In some embodiments, fragmentation comprises treating the sample polynucleotides with one or more restriction endonucleases. Fragmentation can produce fragments having 5′ overhangs, 3′ overhangs, blunt ends, or a combination thereof. In some embodiments, such as when fragmentation comprises the use of one or more restriction endonucleases, cleavage of sample polynucleotides leaves overhangs having a predictable sequence. Fragmented polynucleotides may be subjected to a step of size selecting the fragments via standard methods such as column purification or isolation from an agarose gel.

According to some embodiments, polynucleotides among the plurality of polynucleotides from a sample are circularized. Circularization can include joining the 5′ end of a polynucleotide to the 3′ end of the same polynucleotide, to the 3′ end of another polynucleotide in the sample, or to the 3′ end of a polynucleotide from a different source (e.g. an artificial polynucleotide, such as an oligonucleotide adapter). In some embodiments, the 5′ end of a polynucleotide is joined to the 3′ end of the same polynucleotide (also referred to as “self-joining”). In some embodiment, conditions of the circularization reaction are selected to favor self-joining of polynucleotides within a particular range of lengths, so as to produce a population of circularized polynucleotides of a particular average length. For example, circularization reaction conditions may be selected to favor self-joining of polynucleotides shorter than about 5000, 2500, 1000, 750, 500, 400, 300, 200, 150, 100, 50, or fewer nucleotides in length. In some embodiments, fragments having lengths between 50-5000 nucleotides, 100-2500 nucleotides, or 150-500 nucleotides are favored, such that the average length of circularized polynucleotides falls within the respective range. In some embodiments, 80% or more of the circularized fragments are between 50-500 nucleotides in length, such as between 50-200 nucleotides in length. Reaction conditions that may be optimized include the length of time allotted for a joining reaction, the concentration of various reagents, and the concentration of polynucleotides to be joined. In some embodiments, a circularization reaction preserves the distribution of fragment lengths present in a sample prior to circularization. For example, one or more of the mean, median, mode, and standard deviation of fragment lengths in a sample before circularization and of circularized polynucleotides are within 75%, 80%, 85%, 90%, 95%, or more of one another.

In some cases, rather than preferentially forming self-joining circularization products, one or more adapter oligonucleotides are used, such that the 5′ end and 3′ end of a polynucleotide in the sample are joined by way of one or more intervening adapter oligonucleotides to form a circular polynucleotide. For example, the 5′ end of a polynucleotide can be joined to the 3′ end of an adapter, and the 5′ end of the same adapter can be joined to the 3′ end of the same polynucleotide. An adapter oligonucleotide includes any oligonucleotide having a sequence, at least a portion of which is known, that can be joined to a sample polynucleotide. Adapter oligonucleotides can comprise DNA, RNA, nucleotide analogues, non-canonical nucleotides, labeled nucleotides, modified nucleotides, or combinations thereof. Adapter oligonucleotides can be single-stranded, double-stranded, or partial duplex. In general, a partial-duplex adapter comprises one or more single-stranded regions and one or more double-stranded regions. Double-stranded adapters can comprise two separate oligonucleotides hybridized to one another (also referred to as an “oligonucleotide duplex”), and hybridization may leave one or more blunt ends, one or more 3′ overhangs, one or more 5′ overhangs, one or more bulges resulting from mismatched and/or unpaired nucleotides, or any combination of these. When two hybridized regions of an adapter are separated from one another by a non-hybridized region, a “bubble” structure results. Adapters of different kinds can be used in combination, such as adapters of different sequences. Different adapters can be joined to sample polynucleotides in sequential reactions or simultaneously. In some embodiments, identical adapters are added to both ends of a target polynucleotide. For example, first and second adapters can be added to the same reaction. Adapters can be manipulated prior to combining with sample polynucleotides. For example, terminal phosphates can be added or removed.

Where adapter oligonucleotides are used, the adapter oligonucleotides can contain one or more of a variety of sequence elements, including but not limited to, one or more amplification primer annealing sequences or complements thereof, one or more sequencing primer annealing sequences or complements thereof, one or more barcode sequences, one or more common sequences shared among multiple different adapters or subsets of different adapters, one or more restriction enzyme recognition sites, one or more overhangs complementary to one or more target polynucleotide overhangs, one or more probe binding sites (e.g. for attachment to a sequencing platform, such as a flow cell for massive parallel sequencing, such as flow cells as developed by Illumina, Inc.), one or more random or near-random sequences (e.g. one or more nucleotides selected at random from a set of two or more different nucleotides at one or more positions, with each of the different nucleotides selected at one or more positions represented in a pool of adapters comprising the random sequence), and combinations thereof. In some cases, the adapters may be used to purify those circles that contain the adapters, for example by using beads (particularly magnetic beads for ease of handling) that are coated with oligonucleotides comprising a complementary sequence to the adapter, that can “capture” the closed circles with the correct adapters by hybridization thereto, wash away those circles that do not contain the adapters and any unligated components, and then release the captured circles from the beads. In addition, in some cases, the complex of the hybridized capture probe and the target circle can be directly used to generate concatemers, such as by direct rolling circle amplification (RCA). In some embodiments, the adapters in the circles can also be used as a sequencing primer. Two or more sequence elements can be non-adjacent to one another (e.g. separated by one or more nucleotides), adjacent to one another, partially overlapping, or completely overlapping. For example, an amplification primer annealing sequence can also serve as a sequencing primer annealing sequence. Sequence elements can be located at or near the 3′ end, at or near the 5′ end, or in the interior of the adapter oligonucleotide. A sequence element may be of any suitable length, such as about or less than about 3, 4, 5, 6, 7, 8, 9, 10, 15, 20, 25, 30, 35, 40, 45, 50 or more nucleotides in length. Adapter oligonucleotides can have any suitable length, at least sufficient to accommodate the one or more sequence elements of which they are comprised. In some embodiments, adapters are about or less than about 10, 15, 20, 25, 30, 35, 40, 45, 50, 55, 60, 65, 70, 75, 80, 90, 100, 200, or more nucleotides in length. In some embodiments, an adapter oligonucleotide is in the range of about 12 to 40 nucleotides in length, such as about 15 to 35 nucleotides in length.

In some embodiments, the adapter oligonucleotides joined to fragmented polynucleotides from one sample comprise one or more sequences common to all adapter oligonucleotides and a barcode that is unique to the adapters joined to polynucleotides of that particular sample, such that the barcode sequence can be used to distinguish polynucleotides originating from one sample or adapter joining reaction from polynucleotides originating from another sample or adapter joining reaction. In some embodiments, an adapter oligonucleotide comprises a 5′ overhang, a 3′ overhang, or both that is complementary to one or more target polynucleotide overhangs. Complementary overhangs can be one or more nucleotides in length, including but not limited to 1, 2, 3, 4, 5, 6, 7, 8, 9, 10, 11, 12, 13, 14, 15, or more nucleotides in length. Complementary overhangs may comprise a fixed sequence. Complementary overhangs of an adapter oligonucleotide may comprise a random sequence of one or more nucleotides, such that one or more nucleotides are selected at random from a set of two or more different nucleotides at one or more positions, with each of the different nucleotides selected at one or more positions represented in a pool of adapters with complementary overhangs comprising the random sequence. In some embodiments, an adapter overhang is complementary to a target polynucleotide overhang produced by restriction endonuclease digestion. In some embodiments, an adapter overhang consists of an adenine or a thymine.

A variety of methods for circularizing polynucleotides are available. In some embodiments, circularization comprises an enzymatic reaction, such as use of a ligase (e.g. an RNA or DNA ligase). A variety of ligases are available, including, but not limited to, Circligase™ (Epicentre; Madison, WI), RNA ligase, T4 RNA Ligase 1 (ssRNA Ligase, which works on both DNA and RNA). In addition, T4 DNA ligase can also ligate ssDNA if no dsDNA templates are present, although this is generally a slow reaction. Other non-limiting examples of ligases include NAD-dependent ligases including Taq DNA ligase, Thermus filiformis DNA ligase, Escherichia coli DNA ligase, Tth DNA ligase, Thermus scotoductus DNA ligase (I and II), thermostable ligase, Ampligase thermostable DNA ligase, VanC-type ligase, 9° N DNA Ligase, Tsp DNA ligase, and novel ligases discovered by bioprospecting; ATP-dependent ligases including T4 RNA ligase, T4 DNA ligase, T3 DNA ligase, T7 DNA ligase, Pfu DNA ligase, DNA ligase 1, DNA ligase III, DNA ligase IV, and novel ligases discovered by bioprospecting; and wild-type, mutant isoforms, and genetically engineered variants thereof. Where self-joining is desired, the concentration of polynucleotides and enzyme can be adjusted to facilitate the formation of intramolecular circles rather than intermolecular structures. Reaction temperatures and times can be adjusted as well. In some embodiments, 60° C. is used to facilitate intramolecular circles. In some embodiments, reaction times are between 12-16 hours. Reaction conditions may be those specified by the manufacturer of the selected enzyme. In some embodiments, an exonuclease step can be included to digest any unligated nucleic acids after the circularization reaction. That is, closed circles do not contain a free 5′ or 3′ end, and thus the introduction of a 5′ or 3′ exonuclease will not digest the closed circles but will digest the unligated components. This may find particular use in multiplex systems.

In general, joining ends of a polynucleotide to one-another to form a circular polynucleotide (either directly, or with one or more intermediate adapter oligonucleotides) produces a junction having a junction sequence. Where the 5′ end and 3′ end of a polynucleotide are joined via an adapter polynucleotide, the term “junction” can refer to a junction between the polynucleotide and the adapter (e.g. one of the 5′ end junction or the 3′ end junction), or to the junction between the 5′ end and the 3′ end of the polynucleotide as formed by and including the adapter polynucleotide. Where the 5′ end and the 3′ end of a polynucleotide are joined without an intervening adapter (e.g. the 5′ end and 3′ end of a single-stranded DNA), the term “junction” refers to the point at which these two ends are joined. A junction may be identified by the sequence of nucleotides comprising the junction (also referred to as the “junction sequence”). In some embodiments, samples comprise polynucleotides having a mixture of ends formed by natural degradation processes (such as cell lysis, cell death, and other processes by which DNA is released from a cell to its surrounding environment in which it may be further degraded, such as in cell-free polynucleotides, such as cell-free DNA and cell-free RNA), fragmentation that is a byproduct of sample processing (such as fixing, staining, and/or storage procedures), and fragmentation by methods that cleave DNA without restriction to specific target sequences (e.g. mechanical fragmentation, such as by sonication; non-sequence specific nuclease treatment, such as DNase I, fragmentase). Where samples comprise polynucleotides having a mixture of ends, the likelihood that two polynucleotides will have the same 5′ end or 3′ end is low, and the likelihood that two polynucleotides will independently have both the same 5′ end and 3′ end is extremely low. Accordingly, in some embodiments, junctions may be used to distinguish different polynucleotides, even where the two polynucleotides comprise a portion having the same target sequence. Where polynucleotide ends are joined without an intervening adapter, a junction sequence may be identified by alignment to a reference sequence. For example, where the order of two component sequences appears to be reversed with respect to the reference sequence, the point at which the reversal appears to occur may be an indication of a junction at that point. Where polynucleotide ends are joined via one or more adapter sequences, a junction may be identified by proximity to the known adapter sequence, or by alignment as above if a sequencing read is of sufficient length to obtain sequence from both the 5′ and 3′ ends of the circularized polynucleotide. In some embodiments, the formation of a particular junction is a sufficiently rare event such that it is unique among the circularized polynucleotides of a sample.

Three non-limiting examples of methods of circularizing polynucleotides include the following. In some cases, the polynucleotides are circularized in the absence of adapters, in another case adapters are used, and in another case two adapters are used. Where two adapters are used, one can be joined to the 5′ end of the polynucleotide while the second adapter can be joined to the 3′ end of the same polynucleotide. In some embodiments, adapter ligation may comprise use of two different adapters along with a “splint” nucleic acid that is complementary to the two adapters to facilitate ligation. Forked or “Y” adapters may also be used. Where two adapters are used, polynucleotides having the same adapter at both ends may be removed in subsequent steps due to self-annealing. In additional embodiments of methods according to the present disclosure polynucleotides are circularized in the absence of adapters or in the presence of adapters. Circularized polynucleotides with adapters can be amplified by rolling circle amplification (RCA) using target specific primers or primers which hybridize to the adapter sequences.

Further non-limiting example methods of circularizing polynucleotides, such as single-stranded DNA are described below. The adapter can be asymmetrically added to either the 5′ or 3′ end of a polynucleotide. In some cases, the single-stranded DNA (ssDNA) can have a free hydroxyl group at the 3′ end, and the adapter can have a blocked 3′ end such that in the presence of a ligase, a preferred reaction joins the 3′ end of the ssDNA to the 5′ end of the adapter. In this embodiment, it can be useful to use agents such as polyethylene glycols (PEGs) to drive the intermolecular ligation of a single ssDNA fragment and a single adapter, prior to an intramolecular ligation to form a circle. The reverse order of ends can also be done (blocked 3′, free 5′, etc.). Once the linear ligation is accomplished, the ligated pieces can be treated with an enzyme to remove the blocking moiety, such as through the use of a kinase or other suitable enzymes or chemistries. Once the blocking moiety is removed, the addition of a circularization enzyme, such as CircLigase, allows an intramolecular reaction to form the circularized polynucleotide. In some cases, by using a double-stranded adapter with one strand having a 5′ or 3′ end blocked, a double stranded structure can be formed, which upon ligation produces a double-stranded fragment with nicks. The two strands can then be separated, the blocking moiety removed, and the single-stranded fragment circularized to form a circularized polynucleotide. In some cases, double-stranded DNA (dsDNA) is circularized to yield a circularized, double-stranded circle. The double-stranded circle can be denatured to allow for primer binding and amplification of both strands.

In some embodiments, molecular clamps are used to bring two ends of a polynucleotide (e.g. a single-stranded DNA) together in order to enhance the rate of intramolecular circularization. An example illustration of one such process is provided in FIG. 5. This can be done with or without adapters. The use of molecular clamps may be particularly useful in cases where the average polynucleotide fragment is greater than about 100 nucleotides in length. In some embodiments, the molecular clamp probe comprises three domains: a first domain, an intervening domain, and a second domain. The first and second domains will hybridize to corresponding sequences in a target polynucleotide via sequence complementarity. The intervening domain of the molecular clamp probe may not significantly hybridize with the target sequence. The hybridization of the clamp with the target polynucleotide thus can bring the two ends of the target sequence into closer proximity, which facilitates the intramolecular circularization of the target sequence in the presence of a circularization enzyme. In some embodiments, this is additionally useful as the molecular clamp can serve as an amplification primer as well.

After circularization, ligation enzymes are removed from reaction products using a protein degradation step. In some embodiments, protein degradation comprises treatment to remove or degrade ligase used in the circularization reaction. In some embodiments, treatment to degrade ligase comprises treatment with a protease, such as proteinase K. Proteinase K treatment may follow manufacturer protocols or standard protocols (e.g. as provided in Sambrook and Green, Molecular Cloning: A Laboratory Manual, 4th Edition (2012)). In some embodiments, protein degradation comprises treatment with a low pH or acidic solution or buffer. In some embodiments, protein degradation comprises heating the reaction, for example heating the reaction above 55° C., above 60° C., above 65° C., above 70° C., or greater. In some embodiments, linear polynucleotides are degraded, after circularization. In some embodiments, linear polynucleotides are degraded using an exonuclease. In some embodiments, the exonuclease comprises a lambda exonuclease. In some embodiments, the exonuclease comprises a RecJf nuclease. In some embodiments, an exonuclease is selected from at least one of ExoI, ExoIII, ExoV, ExoVII, and ExoT.

Circularization may be followed directly by sequencing the circularized polynucleotides. Alternatively, sequencing may be preceded by one or more amplification reactions. In general, “amplification” refers to a process by which one or more copies are made of a target polynucleotide or a portion thereof. A variety of methods of amplifying polynucleotides (e.g. DNA and/or RNA) are available. Amplification may be linear, exponential, or involve both linear and exponential phases in a multi-phase amplification process. Amplification methods may involve changes in temperature, such as a heat denaturation step, or may be isothermal processes that do not require heat denaturation. The polymerase chain reaction (PCR) uses multiple cycles of denaturation, annealing of primer pairs to opposite strands, and primer extension to exponentially increase copy numbers of the target sequence. Denaturation of annealed nucleic acid strands may be achieved by the application of heat, increasing local metal ion concentrations (e.g. U.S. Pat. No. 6,277,605), ultrasound radiation (e.g. WO/2000/049176), application of voltage (e.g. U.S. Pat. Nos. 5,527,670, 6,033,850, 5,939,291, and 6,333,157), and application of an electromagnetic field in combination with primers bound to a magnetically-responsive material (e.g. U.S. Pat. No. 5,545,540). In a variation called RT-PCR, reverse transcriptase (RT) is used to make a complementary DNA (cDNA) from RNA, and the cDNA is then amplified by PCR to produce multiple copies of DNA (e.g. U.S. Pat. Nos. 5,322,770 and 5,310,652). One example of an isothermal amplification method is strand displacement amplification, commonly referred to as SDA, which uses cycles of annealing pairs of primer sequences to opposite strands of a target sequence, primer extension in the presence of a dNTP to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product (e.g. U.S. Pat. Nos. 5,270,184 and 5,455,166). Thermophilic SDA (tSDA) uses thermophilic endonucleases and polymerases at higher temperatures in essentially the same method (European Pat. No. 0 684 315). Other amplification methods include rolling circle amplification (RCA) (e.g., Lizardi, “Rolling Circle Replication Reporter Systems,” U.S. Pat. No. 5,854,033); helicase dependent amplification (HDA) (e.g., Kong et al., “Helicase Dependent Amplification Nucleic Acids,” U.S. Pat. Appln. Pub. No. US 2004-0058378 A1); and loop-mediated isothermal amplification (LAMP) (e.g., Notomi et al., “Process for Synthesizing Nucleic Acid,” U.S. Pat. No. 6,410,278). In some cases, isothermal amplification utilizes transcription by an RNA polymerase from a promoter sequence, such as may be incorporated into an oligonucleotide primer. Transcription-based amplification methods include nucleic acid sequence based amplification, also referred to as NASBA (e.g. U.S. Pat. No. 5,130,238); methods which rely on the use of an RNA replicase to amplify the probe molecule itself, commonly referred to as Qβ replicase (e.g., Lizardi, P. et al. (1988) BioTechnol. 6, 1197-1202); self-sustained sequence replication (e.g., Guatelli, J. et al. (1990) Proc. Natl. Acad. Sci. USA 87, 1874-1878; Landgren (1993) Trends in Genetics 9, 199-202; and HELEN H. LEE et al. NUCLEIC ACID AMPLIFICATION TECHNOLOGIES (1997)); and methods for generating additional transcription templates (e.g. U.S. Pat. Nos. 5,480,784 and 5,399,491). Further methods of isothermal nucleic acid amplification include the use of primers containing non-canonical nucleotides (e.g. uracil or RNA nucleotides) in combination with an enzyme that cleaves nucleic acids at the non-canonical nucleotides (e.g. DNA glycosylase or RNaseH) to expose binding sites for additional primers (e.g. U.S. Pat. Nos. 6,251,639, 6,946,251, and 7,824,890). Isothermal amplification processes can be linear or exponential.

In some embodiments, amplification comprises rolling circle amplification (RCA). A typical RCA reaction mixture comprises one or more primers, a polymerase, and dNTPs, and produces concatemers. Typically, the polymerase in an RCA reaction is a polymerase having strand-displacement activity. A variety of such polymerases are available, non-limiting examples of which include exonuclease minus DNA Polymerase I large (Klenow) Fragment, Phi29 DNA polymerase, Taq DNA Polymerase, and the like. In general, a concatemer is a polynucleotide amplification product comprising two or more copies of a target sequence from a template polynucleotide (e.g. about or more than about 2, 3, 4, 5, 6, 7, 8, 9, 10, or more copies of the target sequence; in some embodiments, about or more than about 2 copies). Amplification primers may be of any suitable length, such as about or at least about 5, 10, 15, 20, 25, 30, 35, 40, 45, 50, 55, 60, 65, 70, 75, 80, 90, 100, or more nucleotides, any portion or all of which may be complementary to the corresponding target sequence to which the primer hybridizes (e.g. about, or at least about 5, 10, 15, 20, 25, 30, 35, 40, 45, 50, or more nucleotides). Three non-limiting examples of suitable primers include the following. In some cases, the use of no adapters and a target specific primer can be used for the detection of the presence or absence of a sequence variant within specific target sequences. In some embodiments, multiple target-specific primers for a plurality of targets are used in the same reaction. For example, target-specific primers for about or at least about 10, 50, 100, 150, 200, 250, 300, 400, 500, 1000, 2500, 5000, 10000, 15000, or more different target sequences may be used in a single amplification reaction in order to amplify a corresponding number of target sequences (if present) in parallel. Multiple target sequences may correspond to different portions of the same gene, different genes, or non-gene sequences. Where multiple primers target multiple target sequences in a single gene, primers may be spaced along the gene sequence (e.g. spaced apart by about or at least about 50 nucleotides, every 50-150 nucleotides, or every 50-100 nucleotides) in order to cover all or a specified portion of a target gene. In some cases, a primer that hybridizes to an adapter sequence (which in some cases may be an adapter oligonucleotide itself) is used.

In some cases, amplification is effected by random primers. In general, a random primer comprises one or more random or near-random sequences (e.g. one or more nucleotides selected at random from a set of two or more different nucleotides at one or more positions, with each of the different nucleotides selected at one or more positions represented in a pool of adapters comprising the random sequence). In this way, polynucleotides (e.g. all or substantially all circularized polynucleotides) can be amplified in a sequence non-specific fashion. Such procedures may be referred to as “whole genome amplification” (WGA); however, typical WGA protocols (which do not involve a circularization step) do not efficiently amplify short polynucleotides, such as polynucleotide fragments contemplated by the present disclosure. For further illustrative discussion of WGA procedures, see for example Li et al (2006) J Mol. Diagn. 8(1):22-30.

Where circularized polynucleotides are amplified prior to sequencing, amplified products may be subjected to sequencing directly without enrichment, or subsequent to one or more enrichment steps. Enrichment may comprise purifying one or more reaction components, such as by retention of amplification products or removal of one or more reagents. For example, amplification products may be purified by hybridization to a plurality of probes attached to a substrate, followed by release of captured polynucleotides, such as by a washing step. Alternatively, amplification products can be labeled with a member of a binding pair followed by binding to the other member of the binding pair attached to a substrate, and washing to release the amplification product. Possible substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica, or silica-based materials including silicon and modified silicon, carbon, metals, inorganic glasses, plastics, optical fiber bundles, and a variety of other polymers. In some embodiments, the substrate is in the form of a bead or other small, discrete particle, which may be a magnetic or paramagnetic bead to facilitate isolation through application of a magnetic field. In general, “binding pair” refers to one of a first and a second moiety, wherein the first and the second moiety have a specific binding affinity for each other. Suitable binding pairs include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine); biotin/avidin (or biotin/streptavidin); calmodulin binding protein (CBP)/calmodulin; hormone/hormone receptor; lectin/carbohydrate; peptide/cell membrane receptor; protein A/antibody; hapten/antihapten; enzyme/cofactor; and enzyme/substrate.

In some embodiments, enrichment following amplification of circularized polynucleotides comprises one or more additional amplification reactions. In some embodiments, enrichment comprises amplifying a target sequence comprising sequence A and sequence B (oriented in a 5′ to 3′ direction) in an amplification reaction mixture comprising (a) the amplified polynucleotide; (b) a first primer comprising sequence A′, wherein the first primer specifically hybridizes to sequence A of the target sequence via sequence complementarity between sequence A and sequence A′; (c) a second primer comprising sequence B, wherein the second primer specifically hybridizes to sequence B′ present in a complementary polynucleotide comprising a complement of the target sequence via sequence complementarity between B and B′; and (d) a polymerase that extends the first primer and the second primer to produce amplified polynucleotides; wherein the distance between the 5′ end of sequence A and the 3′ end of sequence B of the target sequence is 75nt or less. In an example arrangement of the first and second primer with respect to a target sequence in the context of a single repeat (which will typically not be amplified unless circular) and concatemers comprising multiple copies of the target sequence. Given the orientation of the primers with respect to a monomer of the target sequence, this arrangement may be referred to as “back to back” (B2B) or “inverted” primers. Amplification with B2B primers facilitates enrichment of circular and/or concatemeric amplification products. Moreover, this orientation combined with a relatively smaller footprint (total distance spanned by a pair of primers) permits amplification of a wider variety of fragmentation events around a target sequence, as a junction is less likely to occur between primers than in the arrangement of primers found in a typical amplification reaction (facing one another, spanning a target sequence).

In some embodiments, the distance between the 5′ end of sequence A and the 3′ end of sequence B is about or less than about 200, 150, 100, 75, 50, 40, 30, 25, 20, 15, or fewer nucleotides. In some embodiments, sequence A is the complement of sequence B. In some embodiments, multiple pairs of B2B primers directed to a plurality of different target sequences are used in the same reaction to amplify a plurality of different target sequences in parallel (e.g. about or at least about 10, 50, 100, 150, 200, 250, 300, 400, 500, 1000, 2500, 5000, 10000, 15000, or more different target sequences). Primers can be of any suitable length, such as described elsewhere herein. Amplification may comprise any suitable amplification reaction under appropriate conditions, such as an amplification reaction described herein. In some embodiments, amplification is a polymerase chain reaction.

In some embodiments, B2B primers comprise at least two sequence elements, a first element that hybridizes to a target sequence via sequence complementarity, and a 5′ “tail” that does not hybridize to the target sequence during a first amplification phase at a first hybridization temperature during which the first element hybridizes (e.g. due to lack of sequence complementarity between the tail and the portion of the target sequence immediately 3′ with respect to where the first element binds). For example, the first primer comprises sequence C 5′ with respect to sequence A′, the second primer comprises sequence D 5′ with respect to sequence B, and neither sequence C nor sequence D hybridize to the plurality of concatemers during a first amplification phase at a first hybridization temperature. In some embodiments in which such tailed primers are used, amplification can comprise a first phase and a second phase; the first phase comprises a hybridization step at a first temperature, during which the first and second primers hybridize to the concatemers (or circularized polynucleotides) and primer extension; and the second phase comprises a hybridization step at a second temperature that is higher than the first temperature, during which the first and second primers hybridize to amplification products comprising extended first or second primers, or complements thereof, and primer extension. The higher temperature favors hybridization between the first element and tail element of the primer in primer extension products over shorter fragments formed by hybridization between only the first element in a primer and an internal target sequence within a concatemer. Accordingly, the two-phase amplification may be used to reduce the extent to which short amplification products might otherwise be favored, thereby maintaining a relatively higher proportion of amplification products having two or more copies of a target sequence. For example, after 5 cycles (e.g. at least 5, 6, 7, 8, 9, 10, 15, 20, or more cycles) of hybridization at the second temperature and primer extension, at least 5% (e.g. at least 5%, 6%, 7%, 8%, 9%, 10%, 15%, 20%, 25%, 30%, or more) of amplified polynucleotides in the reaction mixture comprise two or more copies of the target sequence.

In some embodiments, enrichment comprise amplification under conditions that are skewed to increase the length of amplicons from concatemers. For example, the primer concentration can be lowered, such that not every priming site will hybridize a primer, thus making the PCR products longer. Similarly, decreasing the primer hybridization time during the cycles will similarly allow fewer primers to hybridize, thus also making the average PCR amplicon size increase. Furthermore, increasing the temperature and/or extension time of the cycles may similarly increase the average length of the PCR amplicons. Any combination of these techniques can be used.

In some embodiments, particularly where an amplification with B2B primers has been performed, amplification products are treated to filter the resulting amplicons on the basis of size to reduce and/or eliminate the number of monomers a mixture comprising concatemers. This can be done using a variety of available techniques, including, but not limited to, fragment excision from gels and gel filtration (e.g. to enrich for fragments larger than about 300, 400, 500, or more nucleotides in length); as well as SPRI beads (Agencourt AMPure XP) for size selection by fine-tuning the binding buffer concentration. For example, the use of 0.6× binding buffer during mixing with DNA fragments may be used to preferentially bind DNA fragments larger than about 500 base pairs (bp).

In some embodiments, where amplification result in single-stranded concatemers, the single strands are converted to double-stranded constructs either prior to or as part of the formation of sequencing libraries that are generated for sequencing reactions. A variety of suitable methods to generate a double-stranded construct from a single-stranded nucleic acid are available. A number of possible methods are described herein, although a number of other methods can be used as well. In some cases, for example, the use of random primers, polymerase, dNTPs, and a ligase will result in double strands. In some cases, second strand synthesis when the concatemer contains adapter sequences can be used as the primers in the reaction. In some cases, the use of a “loop,” where one terminus of the loop adapter is added to the terminus of the concatemers, wherein the loop adapter has a small section of self-hybridizing nucleic acids. In this case, the ligation of the loop adapter results in the loop that is self-hybridized and serves as the polymerase primer template. In some cases, the use of hyper-branching primers, generally of the most use in cases where the target sequence is known, where multiple strands are formed, particularly when a polymerase with a strong strand displacement function is used.

According to some embodiments, circularized polynucleotides (or amplification products thereof, which may have optionally been enriched) are subjected to a sequencing reaction to generate sequencing reads. Sequencing reads produced by such methods may be used in accordance with other methods disclosed herein. A variety of sequencing methodologies are available, particularly high-throughput sequencing methodologies. Examples include, without limitation, sequencing systems manufactured by Illumina (sequencing systems such as HiSeq® and MiSeq®), Life Technologies (Ion Torrent®, SOLiD®, etc.), Roche's 454 Life Sciences systems, Pacific Biosciences systems, etc. In some embodiments, sequencing comprises use of HiSeq® and MiSeq® systems to produce reads of about or more than about 50, 75, 100, 125, 150, 175, 200, 250, 300, or more nucleotides in length. In some embodiments, sequencing comprises a sequencing by synthesis process, where individual nucleotides are identified iteratively, as they are added to the growing primer extension product. Pyrosequencing is an example of a sequence by synthesis process that identifies the incorporation of a nucleotide by assaying the resulting synthesis mixture for the presence of by-products of the sequencing reaction, namely pyrophosphate. In particular, a primer/template/polymerase complex is contacted with a single type of nucleotide. If that nucleotide is incorporated, the polymerization reaction cleaves the nucleoside triphosphate between the α and β phosphates of the triphosphate chain, releasing pyrophosphate. The presence of released pyrophosphate is then identified using a chemiluminescent enzyme reporter system that converts the pyrophosphate, with AMP, into ATP, then measures ATP using a luciferase enzyme to produce measurable light signals. Where light is detected, the base is incorporated, where no light is detected, the base is not incorporated. Following appropriate washing steps, the various bases are cyclically contacted with the complex to sequentially identify subsequent bases in the template sequence. See, e.g., U.S. Pat. No. 6,210,891.

In related sequencing processes, the primer/template/polymerase complex is immobilized upon a substrate and the complex is contacted with labeled nucleotides. The immobilization of the complex may be through the primer sequence, the template sequence and/or the polymerase enzyme, and may be covalent or noncovalent. For example, immobilization of the complex can be via a linkage between the polymerase or the primer and the substrate surface. In alternate configurations, the nucleotides are provided with and without removable terminator groups. Upon incorporation, the label is coupled with the complex and is thus detectable. In the case of terminator bearing nucleotides, all four different nucleotides, bearing individually identifiable labels, are contacted with the complex. Incorporation of the labeled nucleotide arrests extension, by virtue of the presence of the terminator, and adds the label to the complex, allowing identification of the incorporated nucleotide. The label and terminator are then removed from the incorporated nucleotide, and following appropriate washing steps, the process is repeated. In the case of non-terminated nucleotides, a single type of labeled nucleotide is added to the complex to determine whether it will be incorporated, as with pyrosequencing. Following removal of the label group on the nucleotide and appropriate washing steps, the various different nucleotides are cycled through the reaction mixture in the same process. See, e.g., U.S. Pat. No. 6,833,246, incorporated herein by reference in its entirety for all purposes. For example, the Illumina Genome Analyzer System is based on technology described in WO 98/44151, wherein DNA molecules are bound to a sequencing platform (flow cell) via an anchor probe binding site (otherwise referred to as a flow cell binding site) and amplified in situ on a glass slide. A solid surface on which DNA molecules are amplified typically comprise a plurality of first and second bound oligonucleotides, the first complementary to a sequence near or at one end of a target polynucleotide and the second complementary to a sequence near or at the other end of a target polynucleotide. This arrangement permits bridge amplification, such as described in US20140121116. The DNA molecules are then annealed to a sequencing primer and sequenced in parallel base-by-base using a reversible terminator approach. Hybridization of a sequencing primer may be preceded by cleavage of one strand of a double-stranded bridge polynucleotide at a cleavage site in one of the bound oligonucleotides anchoring the bridge, thus leaving one single strand not bound to the solid substrate that may be removed by denaturing, and the other strand bound and available for hybridization to a sequencing primer. Typically, the Illumina Genome Analyzer System utilizes flow-cells with 8 channels, generating sequencing reads of 18 to 36 bases in length, generating >1.3 Gbp of high quality data per run (see www.illumina.com).

In yet a further sequence by synthesis process, the incorporation of differently labeled nucleotides is observed in real time as template dependent synthesis is carried out. In particular, an individual immobilized primer/template/polymerase complex is observed as fluorescently labeled nucleotides are incorporated, permitting real time identification of each added base as it is added. In this process, label groups are attached to a portion of the nucleotide that is cleaved during incorporation. For example, by attaching the label group to a portion of the phosphate chain removed during incorporation, i.e., α β, γ, or other terminal phosphate group on a nucleoside polyphosphate, the label is not incorporated into the nascent strand, and instead, natural DNA is produced. Observation of individual molecules typically involves the optical confinement of the complex within a very small illumination volume. By optically confining the complex, one creates a monitored region in which randomly diffusing nucleotides are present for a very short period of time, while incorporated nucleotides are retained within the observation volume for longer as they are being incorporated. This results in a characteristic signal associated with the incorporation event, which is also characterized by a signal profile that is characteristic of the base being added. In related aspects, interacting label components, such as fluorescent resonant energy transfer (FRET) dye pairs, are provided upon the polymerase or other portion of the complex and the incorporating nucleotide, such that the incorporation event puts the labeling components in interactive proximity, and a characteristic signal results, that is again, also characteristic of the base being incorporated (See, e.g., U.S. Pat. Nos. 6,917,726, 7,033,764, 7,052,847, 7,056,676, 7,170,050, 7,361,466, and 7,416,844; and US 20070134128).

In some embodiments, the nucleic acids in the sample can be sequenced by ligation. This method typically uses a DNA ligase enzyme to identify the target sequence, for example, as used in the polony method and in the SOLiD technology (Applied Biosystems, now Invitrogen). In general, a pool of all possible oligonucleotides of a fixed length is provided, labeled according to the sequenced position. Oligonucleotides are annealed and ligated; the preferential ligation by DNA ligase for matching sequences results in a signal corresponding to the complementary sequence at that position.

In some embodiments, sequencing libraries are constructed from the amplified DNA concatemers prior to sequencing analysis. The amplified DNA concatemers can be simultaneously fragmented and tagged with sequencing adapters. In some cases, the amplified DNA concatemers are fragmented, for example by sonication, and adapters are added to both ends of the fragments.

According to some embodiments, a sequence difference between sequencing reads and a reference sequence are called as a genuine sequence variant (e.g. existing in the sample prior to amplification or sequencing, and not a result of either of these processes) if it occurs in at least two different polynucleotides (e.g. two different circular polynucleotides, which can be distinguished as a result of having different junctions). Because sequence variants that are the result of amplification or sequencing errors are unlikely to be duplicated exactly (e.g. position and type) on two different polynucleotides comprising the same target sequence, adding this validation parameter greatly reduces the background of erroneous sequence variants, with a concurrent increase in the sensitivity and accuracy of detecting actual sequence variation in a sample. In some embodiments, a sequence variant having a frequency of about or less than about 5%, 4%, 3%, 2%, 1.5%, 1%, 0.75%, 0.5%, 0.25%, 0.1%, 0.075%, 0.05%, 0.04%, 0.03%, 0.02%, 0.01%, 0.005%, 0.001%, or lower is sufficiently above background to permit an accurate call. In some embodiments, the sequence variant occurs with a frequency of about or less than about 0.1%. In some embodiments, the frequency of a sequence variant is sufficiently above background when such frequency is statistically significantly above the background error rate (e.g. with a p-value of about or less than about 0.05, 0.01, 0.001, 0.0001, or lower). In some embodiments, the frequency of a sequence variant is sufficiently above background when such frequency is about or at least about 2-fold, 3-fold, 4-fold, 5-fold, 6-fold, 7-fold, 8-fold, 9-fold, 10-fold, 25-fold, 50-fold, 100-fold, or more above the background error rate (e.g. at least 5-fold higher). In some embodiments, the background error rate in accurately determining the sequence at a given position is about or less than about 1%, 0.5%, 0.1%, 0.05%, 0.01%, 0.005%, 0.001%, 0.0005%, or lower. In some embodiments, the error rate is lower than 0.001%.

In some embodiments, identifying a genuine sequence variant (also referred to as “calling” or “making a call”) comprises optimally aligning one or more sequencing reads with a reference sequence to identify differences between the two, as well as to identify junctions. In general, alignment involves placing one sequence along another sequence, iteratively introducing gaps along each sequence, scoring how well the two sequences match, and preferably repeating for various positions along the reference. The best-scoring match is deemed to be the alignment and represents an inference about the degree of relationship between the sequences. In some embodiments, a reference sequence to which sequencing reads are compared is a reference genome, such as the genome of a member of the same species as the subject. A reference genome may be complete or incomplete. In some embodiments, a reference genome consists only of regions containing target polynucleotides, such as from a reference genome or from a consensus generated from sequencing reads under analysis. In some embodiments, a reference sequence comprises or consists of sequences of polynucleotides of one or more organisms, such as sequences from one or more bacteria, archaea, viruses, protists, fungi, or other organism. In some embodiments, the reference sequence consists of only a portion of a reference genome, such as regions corresponding to one or more target sequences under analysis (e.g. one or more genes, or portions thereof). For example, for detection of a pathogen (such as in the case of contamination detection), the reference genome is the entire genome of the pathogen (e.g. HIV, HPV, or a harmful bacterial strain, e.g. E. coli), or a portion thereof useful in identification, such as of a particular strain or serotype. In some embodiments, sequencing reads are aligned to multiple different reference sequences, such as to screen for multiple different organisms or strains.

In a typical alignment, a base in a sequencing read alongside a non-matching base in the reference indicates that a substitution mutation has occurred at that point. Similarly, where one sequence includes a gap alongside a base in the other sequence, an insertion or deletion mutation (an “indel”) is inferred to have occurred. When it is desired to specify that one sequence is being aligned to one other, the alignment is sometimes called a pairwise alignment. Multiple sequence alignment generally refers to the alignment of two or more sequences, including, for example, by a series of pairwise alignments. In some embodiments, scoring an alignment involves setting values for the probabilities of substitutions and indels. When individual bases are aligned, a match or mismatch contributes to the alignment score by a substitution probability, which could be, for example, 1 for a match and 0.33 for a mismatch. An indel deducts from an alignment score by a gap penalty, which could be, for example, −1. Gap penalties and substitution probabilities can be based on empirical knowledge or a priori assumptions about how sequences mutate. Their values affect the resulting alignment. Examples of algorithms for performing alignments include, without limitation, the Smith-Waterman (SW) algorithm, the Needleman-Wunsch (NW) algorithm, algorithms based on the Burrows-Wheeler Transform (BWT), and hash function aligners such as Novoalign (Novocraft Technologies; available at www.novocraft.com), ELAND (Illumina, San Diego, Calif), SOAP (available at soap.genomics.org.cn), and Maq (available at maq.sourceforge.net). One exemplary alignment program, which implements a BWT approach, is Burrows-Wheeler Aligner (BWA) available from the SourceForge web site maintained by Geeknet (Fairfax, Va.). BWT typically occupies 2 bits of memory per nucleotide, making it possible to index nucleotide sequences as long as 4G base pairs with a typical desktop or laptop computer. The pre-processing includes the construction of BWT (i.e., indexing the reference) and the supporting auxiliary data structures. BWA includes two different algorithms, both based on BWT. Alignment by BWA can proceed using the algorithm bwa-short, designed for short queries up to about 200 by with low error rate (<3%) (Li H. and Durbin R. Bioinformatics, 25:1754-60 (2009)). The second algorithm, BWA-SW, is designed for long reads with more errors (Li H. and Durbin R. (2010). Fast and accurate long-read alignment with Burrows-Wheeler Transform. Bioinformatics, Epub.). The bwa-sw aligner is sometimes referred to as “bwa-long”, “bwa long algorithm”, or similar. An alignment program that implements a version of the Smith-Waterman algorithm is MUMmer, available from the SourceForge web site maintained by Geeknet (Fairfax, Va.). MUMmer is a system for rapidly aligning entire genomes, whether in complete or draft form (Kurtz, S., et al., Genome Biology, 5:R12 (2004); Delcher, A. L., et al., Nucl. Acids Res., 27:11 (1999)). For example, MUMmer 3.0 can find all 20-basepair or longer exact matches between a pair of 5-megabase genomes in 13.7 seconds, using 78 MB of memory, on a 2.4 GHz Linux desktop computer. MUMmer can also align incomplete genomes; it can easily handle the 100s or 1000s of contigs from a shotgun sequencing project, and will align them to another set of contigs or a genome using the NUCmer program included with the system. Other non-limiting examples of alignment programs include: BLAT from Kent Informatics (Santa Cruz, Calif.) (Kent, W. J., Genome Research 4: 656-664 (2002)); SOAP2, from Beijing Genomics Institute (Beijing, Conn.) or BGI Americas Corporation (Cambridge, Mass.); Bowtie (Langmead, et al., Genome Biology, 10:R25 (2009)); Efficient Large-Scale Alignment of Nucleotide Databases (ELAND) or the ELANDv2 component of the Consensus Assessment of Sequence and Variation (CASAVA) software (Illumina, San Diego, Calif.); RTG Investigator from Real Time Genomics, Inc. (San Francisco, Calif.); Novoalign from Novocraft (Selangor, Malaysia); Exonerate, European Bioinformatics Institute (Hinxton, UK) (Slater, G., and Birney, E., BMC Bioinformatics 6:31(2005)), Clustal Omega, from University College Dublin (Dublin, Ireland) (Sievers F., et al., Mol Syst Biol 7, article 539 (2011)); ClustalW or ClustalX from University College Dublin (Dublin, Ireland) (Larkin M. A., et al., Bioinformatics, 23, 2947-2948 (2007)); and FASTA, European Bioinformatics Institute (Hinxton, UK) (Pearson W. R., et al., PNAS 85(8):2444-8 (1988); Lipman, D. J., Science 227(4693):1435-41 (1985)).

In particular for embodiments, 3′ tailing reactions are used. In some cases, cell-free double stranded polynucleotides 1, 2, 3 . . . K, of a sample, each contain a genetic locus consisting of a single nucleotide, which may be occupied by a “G” or a rare variant “A”. A sample containing such polynucleotides may be a patient tissue sample, such as a blood or plasma sample, or the like. Typically, reference sequences (e.g. in human genome databases) are available to compare the polynucleotide sequences to. Each polynucleotide has four sequence regions corresponding to the sequences of the two complementary strands at each end. Thus, for example, a target polynucleotide can have sequence regions n1 and n2 at each end of strand and has complementary sequence regions n1′ and n2′ at the ends of complementary strand. Although sequence regions of the various polynucleotide strands are illustrated as small portions of strands, the sequence regions may comprise the entire segments from the end of a strand to genetic locus.

In some embodiments, to the target polynucleotides of the sample is added a 3′ tailing activity along with nucleic acid monomers and/or other reaction components to implement tailing reaction that extends the 3′ ends with one or more A's. In this embodiment, the extension of predetermined nucleotides is shown as “A . . . A” to indicate that one or more nucleotides are added, but that the exact number added to each strand may be undetermined (unless an exo⁻ polymerase is used, as noted below). The representation of the added nucleotide by “A . . . A” is not intended to limit the kind of added nucleotides to only A's. The added nucleotides are predetermined in the sense that the kind of nucleotide precursors used in a tailing reaction are known and selected as an assay design choice. For example, a factor in the selection of a kind of predetermined nucleotide for a particular embodiment may be the efficiency of the circularization step in view of the kind of nucleotide selected. In some embodiments, nucleotide precursors may be nucleoside triphosphates of any of the four nucleotides, either separately, so that homopolymer tails are produced, or in mixtures, so that bi- or tri-nucleotide tails are produced. In some cases, uracil, and/or nucleotide analogues may be used in addition to or in place of the four natural DNA bases. In some embodiments in which a CircLigase™ enzyme is used, predetermined nucleotides may be A's and/or T's. In some embodiments, an exo⁻ polymerase is used in a tailing reaction, and only a single deoxyadenylate is added to a 3′ end.

After tailing, and optional separation of the reaction products from the reaction mixture, individual strands are circularized using a circularization reaction to produce circles, each comprising a sequence element of the form “nⱼ-A . . . A-nⱼ₊₁”. After circularization, and optional separation of circles from the reaction mixture, primers are annealed to one or more primer binding sites of circles, after which they are extended to produce concatemers each containing copies of their respective nⱼ-A . . . A-nⱼ₊₁ sequence element. After sequencing, complementary strands, such as and, may be identified by matching sequence element components, nⱼ and nⱼ₊₁, with their respective complements, nⱼ′ and nⱼ₊₁′. Selection of primer binding sites on circles is a matter of design choice, or alternatively, random sequence primers may be used. In some embodiments, a single primer binding site is selected adjacent to genetic locus; in other embodiments, a plurality of primer binding sites are selected, each for a separate primer, to ensure amplification even if a boundary happens to occur in one of the primer binding sites. In some embodiments, two primers with separate primer binding sites are used to produce concatemers.

After identification of pairs of concatemers containing complementary strands, the concatemer sequences may be aligned and base calls at matching positions of the two strands may be compared. At some positions of concatemer pairs a base called at a given position in one member of a pair may not be complementary to the base called on the other member of the pair, indicating that an incorrect call has been made due to, for example, amplification error, sequencing error, or the like. In this case, the indeterminacy at the given position may be resolve by examining the base calls at corresponding positions of other copies within the concatemer pair. For example, a base call at the given position may be taken to be a consensus, or a majority, of the base calls made for the individual copies in a pair of concatemers. Other methods for making such determinations would be available to one of ordinary skill in the art, which may be used in place of or in addition to these methods to supplement efforts to resolve base calls when sequence information between complementary strands are not complementary. In some cases, where bases at a specified position in complementary strands originating from the same double-stranded molecule (e.g. as identified by the 3′ and 5′ end sequences) are not complementary, a base call is resolved in favor of the reference sequence to which the sample sequence is compared, such that the difference is not identified as a true sequence variant with respect to such reference sequence.

In other circumstances, the same error may appear in each copy of a target polynucleotide within a concatemer. Such data would suggest that the target polynucleotide was damaged before amplification or sequencing.

In still other circumstances, only a single concatemer may be identified; that is, a concatemer for which no match is found based on boundary information, such as, length of the segment of predetermined nucleotides, sequences of adjacent 3′ and 5′ ends, or the like. In some cases, target polynucleotides comprise single stranded polynucleotide 1 and double stranded polynucleotide 2, each encompassing genetic locus. Predetermined nucleotides (for example, adenylates) may be attached to both polynucleotides 1 and 2 in tailing reaction to form 3′ tailed polynucleotides. As described above, polynucleotides may then be circularized, amplified by RCA, and sequenced to give concatemer sequences. In case an observed variant is common in DNA damage, for example, C to T or G to T, such information from an unpaired concatemer will still be helpful in deciding if it is a true mutation versus just a DNA damage.

In some embodiments, primers each containing a molecular tag, e.g. MT1, MT2, and so on, may be annealed to each single stranded circle at predetermined primer binding sites in order to produce concatemers each with a unique tag. The presence of unique molecular tags will distinguish products of single stranded circles that happen to have the same boundary, or nⱼ-A . . . A-nⱼ₊₁ sequence element. Such tags may also be used for counting molecules to determine copy number variation at a genetic locus, for example, in accordance with methods described in Brenner et al, U.S. Pat. No. 7,537,897, or the like, which is incorporated herein by reference. In some embodiments, primers with molecular tags may be selected that have binding sites only on one strand of a target polynucleotide so that concatemers with molecular tags represent only one of the two strands of a target polynucleotide. In other embodiments, circles from complementary strands of a target polynucleotide may each be amplified using a primer having a molecular tag.

In some embodiments, the above steps for identifying complementary strands of target polynucleotides may be incorporated in a method for detecting rare variants at a genetic locus. In some embodiments, the method comprises the following steps: (a) extending by one or more predetermined nucleotides 3′ ends of the polynucleotides; (b) circularizing individual strands of the polynucleotides to form single stranded polynucleotide circles, the one or more predetermined nucleotides defining a boundary between 3′ sequences and 5′ sequences of each single stranded polynucleotide circle; (c) amplifying by rolling circle replication (RCR) the single stranded polynucleotide circles to form concatemers; (d) sequencing the concatemers; (e) identifying pairs of concatemers containing complementary strands of polynucleotides by the identity of 3′ sequences and 5′ sequences adjacent to the one or more predetermined nucleotides; and (f) determining the sequence of the genetic locus from the sequences of the pairs of concatemers comprising complementary strands of the same polynucleotide. In other embodiments, the step of amplifying by RCR the single stranded circles includes annealing a primer having a 5′-noncomplementary tail to the single stranded circles wherein such primer includes a unique molecular tag in the 5′-noncomplementary tail and extending such primer in accordance with an RCR protocol. The resulting product is a concatemer containing a unique molecular tag, which may be counted along with other molecular tags attached to circles from the same locus to provide a copy number measurement for the locus.

In some embodiments, the step of extending may be implemented by tailing by one or more predetermined nucleotides 3′ ends of the polynucleotides in a tailing reaction. In some embodiments, such tailing may be implemented by an untemplated 3′ nucleotide addition activity, such as a TdT activity, an exo− polymerase activity, or the like.

Using the steps described above, concatemer sequences can be identified from polynucleotide sequences. In large-scale-parallel-sequencing (also referred to as “next generation sequencing” or NGS), reads containing concatemers can be identified and used to perform error correction and find sequence variants. Junctions of the original input molecules (the start and the end of the DNA/RNA sequence) can be reconstructed from the concatemers by aligning them to reference sequences; and the junctions can be used to identify the original input molecule and to remove sequencing duplicates for more accurate counting. The strand identity of each read which may contain a concatemer can be computed by aligning the reads to reference sequences and checking the sequence element components, nⱼ and nⱼ₊₁. Variants found in both concatemers labeled as complementary strands have a higher statistical confidence level, which can be used to perform further error correction. Variant confirmation using strand identity may be carried out by (but is not limited to) the following steps: a) variants found in reads with complementary strand identities are considered more confident; b) reads carrying variants can be grouped by its junction identification, the variants are more confident when complementary strand identities are found in reads within a group of reads having the same junction identification; c) reads carrying variants can be grouped by their molecular barcodes or the combination of molecular barcodes and junction identifications. The variants are more confident when the complement strand identities are found in reads within a group of reads having the same molecular barcodes and/or junction identifications.

Error correction using molecular barcodes and junction identification can be used independently, or combined with the error correction with concatemer sequencing as described in the previous steps. a) Reads with different molecular barcodes (or junction identifications) can be grouped into different read families which represents reads originated from different input molecules; b) consensus sequences can be built from the family of reads; c) consensus can be used for variant calling; d) molecular barcodes and junction identifications can be combined to form a composite ID for reads, which will help identify the original input molecules. In some embodiments, a base call (e.g. a sequence difference with respect to a reference sequence) found in different read families are assigned a higher confidence. In some cases, a sequence difference is only identified as a true sequence variant representative of the original source polynucleotide (as opposed to an error of sample processing or analysis) if the sequence difference passes one or more filters that increase confidence of a base call, such as those described above. In some embodiments, a sequence difference is only identified as a true sequence variant if (a) it is identified on both strands of a double-stranded input molecule; (b) it occurs in the consensus sequence for the concatemer from which it originates (e.g. more than 50%, 80%, 90% or more of the repeats within the concatemer contain the sequence difference); and/or (c) it occurs in two different molecules (e.g. as identified by different 3′ and 5′ endpoints, and/or by an exogenous tag sequence).

Determining strand identity: 1) junctions of the original input molecules can be reconstructed from reads which may contain concatemer sequences by aligning the sequences to reference sequences; 2) the junctions can be located in the reads using the alignments; 3) the sequence element component, nⱼ and nⱼ₊₁, which represents the strand identity, can be extracted from the sequence based the junction locations in the reads; and in the case of concatemer, the sequence can be found between the junctions in the concatemer sequences; 4) the strand (positive or negative) of the reference sequence that the reads align to, combined with the strand identity sequences within the reads identified in step 3, can be used to identify the original strand that was incorporated into the sequence library and sequenced, and to identify which strand a sequence variant originated from. For example, suppose a strand identity sequence “AA” is added to the end of a strand of original input DNA fragment; after sequencing the read of the DNA fragment is aligned to the “+” strand of the reference and the strand identity sequence in the read is “AA”, we know the original input strand is “+”; if the strand identity sequence is “TT”, the read is reverse complementary to the original input strand and the original input strand is “−” strand. The strand identity determination allows a sequence variant to be distinguished from its reverse complementary counterpart, for example, C>T substitution from G>A substitution. The precise identification of allele changes can be used to carry out allele-specific error reduction in variant calling. For example, some DNA damage occurs more often as certain allele changes, and allele-specific error reduction can be carried out to suppress such damage; such error reduction can be done by various statistical methods, for example, 1) calculation of distribution of different allele changes in sequencing data (baseline), followed by 2) z-test or other statistical tests to determine if a observed allele change is different from the baseline distribution.

In some embodiments, the present disclosure provides a method of identifying a genetic variant on a particular strand at a genetic locus by comparing the frequency of a measured sequence, or one or more nucleotides, to a baseline frequency of nucleotide damage that results in the same sequence, or one or more nucleotides, as the measured sequence. In some embodiments, such a method may comprise the following steps: (a) extending by one or more predetermined nucleotides 3′ ends of the polynucleotides; (b) amplifying individual strands of the extended polynucleotides; (c) sequencing the amplified individual strands of the extended polynucleotides; (d) identifying complementary strands of polynucleotides by the identity of 3′ sequences and/or 5′ sequences adjacent to the one or more predetermined nucleotides and identifying nucleotides of each strand at the genetic locus; (e) determining a frequency of each of one or more nucleotides at the genetic locus from the identified concatemers for identifying the genetic variant. In some embodiments, this method may be used to distinguish a genetic variant from nucleotide damage by the following step: calling at least one of said one or more nucleotides at said genetic locus on said strand identified by said one or more predetermined nucleotides as said genetic variant whenever said frequency of strands displaying the at least one nucleotide exceeds by a predetermined factor a baseline frequency of strands having nucleotide damage that gives rise to the same nucleotide.

As mentioned above, in some embodiments, the step of amplifying may be carried out by (i) circularizing individual strands of the polynucleotides to form single stranded polynucleotide circles, the one or more predetermined nucleotides defining a boundary between 3′ sequences and 5′ sequences of the polynucleotides in each single stranded polynucleotide circle; and (ii) amplifying by rolling circle replication the single stranded polynucleotide circles to form concatemers of the single stranded polynucleotide circles.

A baseline frequency of strands having nucleotide damage may be based on prior measurements on samples from the same individual who is being tested by the method, or a baseline frequency may be based on prior measurements on a population of individuals other than the individual being tested. A baseline frequency may also depend on and/or be specific for the kind of steps or protocol used in preparing a sample for analysis by a method of the disclosure. By comparing measured frequencies with baseline frequencies a statistical measure may be obtained of a likelihood (or confidence level) that a measured or determined sequence is a genuine genetic variant and not damage or error due to processing.

Typically, the sequencing data is acquired from large scale, parallel sequencing reactions. Many of the next generation high-throughput sequencing systems export data as FASTQ files, although other formats may be used. In some embodiments, sequences are analyzed to identify repeat unit length (e.g. the monomer length), the junction formed by circularization, and any true variation with respect to a reference sequence, typically through sequence alignment. Identifying the repeat unit length can include computing the regions of the repeated units, finding the reference loci of the sequences (e.g. when one or more sequences are particularly targeted for amplification, enrichment, and/or sequencing), the boundaries of each repeated region, and/or the number of repeats within each sequencing run. Sequence analysis can include analyzing sequence data for both strands of a duplex. As noted above, in some embodiments, an identical variant that appears the sequences of reads from different polynucleotides from the sample (e.g. circularized polynucleotides having different junctions) is considered a confirmed variant. In some embodiments, a sequence variant may also be considered a confirmed, or genuine, variant if it occurs in more than one repeated unit of the same polynucleotide, as the same sequence variation is likewise unlikely to occur at the same position in a repeated target sequence within the same concatemer. The quality score of a sequence may be considered in identifying variants and confirmed variants, for example, the sequence and bases with quality scores lower than a threshold may be filtered out. Other bioinformatics methods can be used to further increase the sensitivity and specificity of the variant calls.

In some embodiments, statistical analyses may be applied to determination of variants (mutations) and quantitate the ratio of the variant in total DNA samples. Total measurement of a particular base can be calculated using the sequencing data. For example, from the alignment results calculated in previous steps, one can calculate the number of “effective reads,” that is, number of confirmed reads for each locus. The allele frequency of a variant can be normalized by the effective read count for the locus. The overall noise level, that is the average rate of observed variants across all loci, can be computed. The frequency of a variant and the overall noise level, combined with other factors, can be used to determine the confidence interval of the variant call. Statistical models such as Poisson distributions can be used to assess the confidence interval of the variant calls. The allele frequency of variants can also be used as an indicator of the relative quantity of the variant in the total sample.

In some embodiments, a microbial contaminant is identified based on the calling step. For example, a particular sequence variant may indicate contamination by a potentially infectious microbe. Sequence variants may be identified within a highly conserved polynucleotide for the purpose of identifying a microbe. Exemplary highly conserved polynucleotides useful in the phylogenetic characterization and identification of microbes comprise nucleotide sequences found in the 16S rRNA gene, 23S rRNA gene, 5S rRNA gene, 5.8S rRNA gene, 12S rRNA gene, 18S rRNA gene, 28S rRNA gene, gyrB gene, rpoB gene, fusA gene, recA gene, coxl gene and nifD gene. With eukaryotes, the rRNA gene can be nuclear, mitochondrial, or both. In some embodiments, sequence variants in the 16S-23S rRNA gene internal transcribed spacer (ITS) can be used for differentiation and identification of closely related taxa with or without the use of other rRNA genes. Due to structural constraints of 16S rRNA, specific regions throughout the gene have a highly conserved polynucleotide sequence although non-structural segments may have a high degree of variability. Identifying sequence variants can be used to identify operational taxonomic units (OTUs) that represent a subgenus, a genus, a subfamily, a family, a sub-order, an order, a sub-class, a class, a sub-phylum, a phylum, a sub-kingdom, or a kingdom, and optionally determine their frequency in a population. The detection of particular sequence variants can be used in detecting the presence, and optionally amount (relative or absolute), of a microbe indicative of contamination. Example applications include water quality testing for fecal or other contamination, testing for animal or human pathogens, pinpointing sources of water contamination, testing reclaimed or recycled water, testing sewage discharge streams including ocean discharge plumes, monitoring of aquaculture facilities for pathogens, monitoring beaches, swimming areas or other water related recreational facilities and predicting toxic algal blooms. Food monitoring applications include the periodic testing of production lines at food processing plants, surveying slaughter houses, inspecting the kitchens and food storage areas of restaurants, hospitals, schools, correctional facilities, and other institutions for food borne pathogens such as E. coli strains O157:H7 or 0111:B4, Listeria monocytogenes, or Salmonella enterica subsp. enterica serovar Enteritidis. Shellfish and shellfish producing waters can be surveyed for algae responsible for paralytic shellfish poisoning, neurotoxic shellfish poisoning, diarrhetic shellfish poisoning and amnesic shellfish poisoning. Additionally, imported foodstuffs can be screened while in customs before release to ensure food security. Plant pathogen monitoring applications include horticulture and nursery monitoring for instance the monitoring for Phytophthora ramorum, the microorganism responsible for Sudden Oak Death, crop pathogen surveillance and disease management and forestry pathogen surveillance and disease management. Manufacturing environments for pharmaceuticals, medical devices, and other consumables or critical components where microbial contamination is a major safety concern can be surveyed for the presence of specific pathogens like Pseudomonas aeruginosa, or Staphylococcus aureus, the presence of more common microorganisms associated with humans, microorganisms associated with the presence of water or others that represent the bioburden that was previously identified in that particular environment or in similar ones. Similarly, the construction and assembly areas for sensitive equipment including space craft can be monitored for previously identified microorganism that are known to inhabit or are most commonly introduced into such environments.

In some embodiments, the method comprises identifying a sequence variant in a nucleic acid sample comprising less than 50 ng of polynucleotides, each polynucleotide having a 5′ end and a 3′ end. In some embodiments, the method comprises: (a) circularizing with a ligase individual polynucleotides in said sample to form a plurality of circular polynucleotides; (b) upon separating said ligase from said circular polynucleotides, amplifying the circular polynucleotides to form concatemers; (c) sequencing the concatemers to produce a plurality of sequencing reads; (d) identifying sequence differences between the plurality of sequencing reads and a reference sequence; and (e) calling a sequence difference that occurs with a frequency of 0.05% or higher in said plurality of reads from said nucleic acid sample of less than 50 ng polynucleotides as the sequence variant.

The starting amount of polynucleotides in a sample may be small. In some embodiments, the amount of starting polynucleotides is less than 100 ng. In some embodiments, the amount of starting material is less than 75 ng. In some embodiments, the amount of starting material is less than 50 ng, such as less than 45 ng, 40 ng, 35 ng, 30 ng, 25 ng, 20 ng, 15 ng, 10 ng, 5 ng, 4 ng, 3 ng, 2 ng, 1 ng, 0.5 ng, 0.1 ng, or less. In some embodiments, the amount of starting polynucleotides is in the range of 0.1-100 ng, such as between 1-75 ng, 5-50 ng, or 10-20 ng. In general, lower starting material increases the importance of increased recovery from various processing steps. Processes that reduce the amount of polynucleotides in a sample for participation in a subsequent reaction decrease the sensitivity with which rare mutations can be detected. For example, methods described by Lou et al. (PNAS, 2013, 110 (49)) are expected to recover only 10-20% of the starting material. For large amounts of starting material (e.g. as purified from lab-cultured bacteria), this may not be a substantial obstacle. However, for samples where the starting material is substantially lower, recovery in this low range can be a substantial obstacle to detection of sufficiently rare variants. Accordingly, in some embodiments, sample recovery from one step to another in a method of the disclosure (e.g. the mass fraction of input into a circularization step available for input into a subsequent amplification step or sequencing step) is about or more than about 50%, 60%, 75%, 80%, 85%, 90%, 95%, or more. Recovery from a particular step may be close to 100%. Recovery may be with respect to a particular form, such as recovery of circular polynucleotides from an input of non-circular polynucleotides.

The polynucleotides may be from any suitable sample, such as a sample described herein with respect to the various aspects of the disclosure. Polynucleotides from a sample may be any of a variety of polynucleotides, including but not limited to, DNA, RNA, ribosomal RNA (rRNA), transfer RNA (tRNA), micro RNA (miRNA), messenger RNA (mRNA), fragments of any of these, or combinations of any two or more of these. In some embodiments, samples comprise DNA. In some embodiments, the polynucleotides are single-stranded, either as obtained or by way of treatment (e.g. denaturation). Further examples of suitable polynucleotides are described herein, such as with respect to any of the various aspects of the disclosure. In some embodiments, polynucleotides are subjected to subsequent steps (e.g. circularization and amplification) without an extraction step, and/or without a purification step. For example, a fluid sample may be treated to remove cells without an extraction step to produce a purified liquid sample and a cell sample, followed by isolation of DNA from the purified fluid sample. A variety of procedures for isolation of polynucleotides are available, such as by precipitation or non-specific binding to a substrate followed by washing the substrate to release bound polynucleotides. Where polynucleotides are isolated from a sample without a cellular extraction step, polynucleotides will largely be extracellular or “cell-free” polynucleotides, such as cell-free DNA and cell-free RNA, which may correspond to dead or damaged cells. The identity of such cells may be used to characterize the cells or population of cells from which they are derived, such as in a microbial community. If a sample is treated to extract polynucleotides, such as from cells in a sample, a variety of extraction methods are available, examples of which are provided herein (e.g. with regard to any of the various aspects of the disclosure).

The sequence variant in the nucleic acid sample can be any of a variety of sequence variants. Multiple non-limiting examples of sequence variants are described herein, such as with respect to any of the various aspects of the disclosure. In some embodiments the sequence variant is a single nucleotide polymorphism (SNP). In some embodiments, the sequence variant occurs with a low frequency in the population (also referred to as a “rare” sequence variant). For example, the sequence variant may occur with a frequency of about or less than about 5%, 4%, 3%, 2%, 1.5%, 1%, 0.75%, 0.5%, 0.25%, 0.1%, 0.075%, 0.05%, 0.04%, 0.03%, 0.02%, 0.01%, 0.005%, 0.001%, or lower. In some embodiments, the sequence variant occurs with a frequency of about or less than about 0.1%.

According to some embodiments, polynucleotides of a sample are circularized, such as by use of a ligase. Circularization can include joining the 5′ end of a polynucleotide to the 3′ end of the same polynucleotide, to the 3′ end of another polynucleotide in the sample, or to the 3′ end of a polynucleotide from a different source (e.g. an artificial polynucleotide, such as an oligonucleotide adapter). In some embodiments, the 5′ end of a polynucleotide is joined to the 3′ end of the same polynucleotide (also referred to as “self-joining”). Non-limiting examples of circularization processes (e.g. with and without adapter oligonucleotides), reagents (e.g. types of adapters, use of ligases), reaction conditions (e.g. favoring self-joining), and optional additional processing (e.g. post-reaction purification) are provided herein, such as with regard to any of the various aspects of the disclosure.

As previously described, joining ends of a polynucleotide to one-another to form a circular polynucleotide (either directly, or with one or more intermediate adapter oligonucleotides) generally produces a junction having a junction sequence. Where the 5′ end and 3′ end of a polynucleotide are joined via an adapter polynucleotide, the term “junction” can refer to a junction between the polynucleotide and the adapter (e.g. one of the 5′ end junction or the 3′ end junction), or to the junction between the 5′ end and the 3′ end of the polynucleotide as formed by and including the adapter polynucleotide. Where the 5′ end and the 3′ end of a polynucleotide are joined without an intervening adapter (e.g. the 5′ end and 3′ end of a single-stranded DNA), the term “junction” refers to the point at which these two ends are joined. A junction may be identified by the sequence of nucleotides comprising the junction (also referred to as the “junction sequence”). In some embodiments, samples comprise polynucleotides having a mixture of ends formed by natural degradation processes (such as cell lysis, cell death, and other processes by which DNA is released from a cell to its surrounding environment in which it may be further degraded, such as in cell-free polynucleotides, such as cell-free DNA), fragmentation that is a byproduct of sample processing (such as fixing, staining, and/or storage procedures), and fragmentation by methods that cleave DNA without restriction to specific target sequences (e.g. mechanical fragmentation, such as by sonication; non-sequence specific nuclease treatment, such as DNase I, fragmentase). Where samples comprise polynucleotides having a mixture of ends, the likelihood that two polynucleotides will have the same 5′ end or 3′ end is low, and the likelihood that two polynucleotides will independently have both the same 5′ end and 3′ end is extremely low. Accordingly, in some embodiments, junctions may be used to distinguish different polynucleotides, even where the two polynucleotides comprise a portion having the same target sequence. Where polynucleotide ends are joined without an intervening adapter, a junction sequence may be identified by alignment to a reference sequence. For example, where the order of two component sequences appears to be reversed with respect to the reference sequence, the point at which the reversal appears to occur may be an indication of a junction at that point. Where polynucleotide ends are joined via one or more adapter sequences, a junction may be identified by proximity to the known adapter sequence, or by alignment as above if a sequencing read is of sufficient length to obtain sequence from both the 5′ and 3′ ends of the circularized polynucleotide. In some embodiments, the formation of a particular junction is a sufficiently rare event such that it is unique among the circularized polynucleotides of a sample.

After circularization, reaction products may be purified prior to amplification or sequencing to increase the relative concentration or purity of circularized polynucleotides available for participating in subsequent steps (e.g. by isolation of circular polynucleotides or removal of one or more other molecules in the reaction). For example, a circularization reaction or components thereof may be treated to remove single-stranded (non-circularized) polynucleotides, such as by treatment with an exonuclease. As a further example, a circularization reaction or portion thereof may be subjected to size exclusion chromatography, whereby small reagents are retained and discarded (e.g. unreacted adapters), or circularization products are retained and released in a separate volume. A variety of kits for cleaning up ligation reactions are available, such as kits provided by Zymo oligo purification kits made by Zymo Research. In some embodiments, purification comprises treatment to remove or degrade ligase used in the circularization reaction, and/or to purify circularized polynucleotides away from such ligase. In some embodiments, treatment to degrade ligase comprises treatment with a protease. Suitable proteases are available from prokaryotes, viruses, and eukaryotes. Examples of proteases include proteinase K (from Tritirachium album), pronase E (from Streptomyces griseus), Bacillus polymyxa protease, theromolysin (from thermophilic bacteria), trypsin, subtilisin, furin, and the like. In some embodiments, the protease is proteinase K. Protease treatment may follow manufacturer protocols, or subjected to standard conditions (e.g. as provided in Sambrook and Green, Molecular Cloning: A Laboratory Manual, 4th Edition (2012)). Protease treatment may also be followed by extraction and precipitation. In one example, circularized polynucleotides are purified by proteinase K (Qiagen) treatment in the presence of 0.1% SDS and 20 mM EDTA, extracted with 1:1 phenol/chloroform and chloroform, and precipitated with ethanol or isopropanol. In some embodiments, precipitation is in ethanol.

As described with respect to other aspects of the disclosure, circularization may be followed directly by sequencing the circularized polynucleotides. Alternatively, sequencing may be preceded by one or more amplification reactions. A variety of methods of amplifying polynucleotides (e.g. DNA and/or RNA) are available. Amplification may be linear, exponential, or involve both linear and exponential phases in a multi-phase amplification process. Amplification methods may involve changes in temperature, such as a heat denaturation step, or may be isothermal processes that do not require heat denaturation. Non-limiting examples of suitable amplification processes are described herein, such as with regard to any of the various aspects of the disclosure. In some embodiments, amplification comprises rolling circle amplification (RCA). As described elsewhere herein, a typical RCA reaction mixture comprises one or more primers, a polymerase, and dNTPs, and produces concatemers. Typically, the polymerase in an RCA reaction is a polymerase having strand-displacement activity. A variety of such polymerases are available, non-limiting examples of which include exonuclease minus DNA Polymerase I large (Klenow) Fragment, Phi29 DNA polymerase, Taq DNA Polymerase, and the like. In general, a concatemer is a polynucleotide amplification product comprising two or more copies of a target sequence from a template polynucleotide (e.g. about or more than about 2, 3, 4, 5, 6, 7, 8, 9, 10, or more copies of the target sequence; in some embodiments, about or more than about 2 copies). Amplification primers may be of any suitable length, such as about or at least about 5, 10, 15, 20, 25, 30, 35, 40, 45, 50, 55, 60, 65, 70, 75, 80, 90, 100, or more nucleotides, any portion or all of which may be complementary to the corresponding target sequence to which the primer hybridizes (e.g. about, or at least about 5, 10, 15, 20, 25, 30, 35, 40, 45, 50, or more nucleotides). Examples of various RCA processes are described herein, such as the use of random primers, target-specific primers, and adapter-targeted primers.

Where circularized polynucleotides are amplified prior to sequencing (e.g. to produce concatemers), amplified products may be subjected to sequencing directly without enrichment, or subsequent to one or more enrichment steps. Non-limiting examples of suitable enrichment processes are described herein, such as with respect to any of the various aspects of the disclosure (e.g. use of B2B primers for a second amplification step). According to some embodiments, circularized polynucleotides (or amplification products thereof, which may have optionally been enriched) are subjected to a sequencing reaction to generate sequencing reads. Sequencing reads produced by such methods may be used in accordance with other methods disclosed herein. A variety of sequencing methodologies are available, particularly high-throughput sequencing methodologies. Examples include, without limitation, sequencing systems manufactured by Illumina (sequencing systems such as HiSeq® and MiSeq®), Life Technologies (Ion Torrent®, SOLiD®, etc.), Roche's 454 Life Sciences systems, Pacific Biosciences systems, etc. In some embodiments, sequencing comprises use of HiSeq® and MiSeq® systems to produce reads of about or more than about 50, 75, 100, 125, 150, 175, 200, 250, 300, or more nucleotides in length. Additional non-limiting examples of amplification platforms and methodologies are described herein, such as with respect to any of the various aspects of the disclosure.

According to some embodiments, a sequence difference between sequencing reads and a reference sequence are called as a genuine sequence variant (e.g. existing in the sample prior to amplification or sequencing, and not a result of either of these processes) if it occurs in at least two different polynucleotides (e.g. two different circular polynucleotides, which can be distinguished as a result of having different junctions or two different polynucleotides having a different 5′ end and/or a different 3′ end). Because sequence variants that are the result of amplification or sequencing errors are unlikely to be duplicated exactly (e.g. position and type) on two different polynucleotides comprising the same target sequence, adding this validation parameter greatly reduces the background of erroneous sequence variants, with a concurrent increase in the sensitivity and accuracy of detecting actual sequence variation in a sample. In some embodiments, a sequence variant having a frequency of about or less than about 5%, 4%, 3%, 2%, 1.5%, 1%, 0.75%, 0.5%, 0.25%, 0.1%, 0.075%, 0.05%, 0.04%, 0.03%, 0.02%, 0.01%, 0.005%, 0.001%, or lower is sufficiently above background to permit an accurate call. In some embodiments, the sequence variant occurs with a frequency of about or less than about 0.1%. In some embodiments, the method comprises calling as a genuine sequence variant, those sequence differences having a frequency in the range of about 0.0005% to about 3%, such as between 0.001%-2%, or 0.01%-1%. In some embodiments, the frequency of a sequence variant is sufficiently above background when such frequency is statistically significantly above the background error rate (e.g. with a p-value of about or less than about 0.05, 0.01, 0.001, 0.0001, or lower). In some embodiments, the frequency of a sequence variant is sufficiently above background when such frequency is about or at least about 2-fold, 3-fold, 4-fold, 5-fold, 6-fold, 7-fold, 8-fold, 9-fold, 10-fold, 25-fold, 50-fold, 100-fold, or more above the background error rate (e.g. at least 5-fold higher). In some embodiments, the background error rate in accurately determining the sequence at a given position is about or less than about 1%, 0.5%, 0.1%, 0.05%, 0.01%, 0.005%, 0.001%, 0.0005%, or lower. In some embodiments, the error rate is lower than 0.001%. Methods for determining frequency and error rate are described herein, such as with regard to any of the various aspects of the disclosure.

In some embodiments, identifying a genuine sequence variant (also referred to as “calling” or “making a call”) comprises optimally aligning one or more sequencing reads with a reference sequence to identify differences between the two, as well as to identify junctions. In general, alignment involves placing one sequence along another sequence, iteratively introducing gaps along each sequence, scoring how well the two sequences match, and preferably repeating for various positions along the reference. The best-scoring match is deemed to be the alignment and represents an inference about the degree of relationship between the sequences. A variety of alignment algorithms and aligners implementing them are available, non-limiting examples of which are described herein, such as with respect to any of the various aspects of the disclosure. In some embodiments, a reference sequence to which sequencing reads are compared is a known reference sequence, such as a reference genome (e.g. the genome of a member of the same species as the subject). A reference genome may be complete or incomplete. In some embodiments, a reference genome consists only of regions containing target polynucleotides, such as from a reference genome or from a consensus generated from sequencing reads under analysis. In some embodiments, a reference sequence comprises or consists of sequences of polynucleotides of one or more organisms, such as sequences from one or more bacteria, archaea, viruses, protists, fungi, or other organism. In some embodiments, the reference sequence consists of only a portion of a reference genome, such as regions corresponding to one or more target sequences under analysis (e.g. one or more genes, or portions thereof). For example, for detection of a pathogen (such as in the case of contamination detection), the reference genome is the entire genome of the pathogen (e.g. HIV, HPV, or a harmful bacterial strain, e.g. E. coli), or a portion thereof useful in identification, such as of a particular strain or serotype. In some embodiments, sequencing reads are aligned to multiple different reference sequences, such as to screen for multiple different organisms or strains. Additional non-limiting examples of reference sequences with respect to which sequence differences may be identified (and sequence variants called) are described herein, such as with respect to any of the various aspects of the disclosure.

In one aspect, the disclosure provides a method of amplifying in a reaction mixture a plurality of different concatemers comprising two or more copies of a target sequence, wherein the target sequence comprises sequence A and sequence B oriented in a 5′ to 3′ direction. In some embodiments, the method comprises subjecting the reaction mixture to a nucleic acid amplification reaction, wherein the reaction mixture comprises: (a) the plurality of concatemers, wherein individual concatemers in the plurality comprise different junctions formed by circularizing individual polynucleotides having a 5′ end and a 3′ end; (b) a first primer comprising sequence A′, wherein the first primer specifically hybridizes to sequence A of the target sequence via sequence complementarity between sequence A and sequence A′; (c) a second primer comprising sequence B, wherein the second primer specifically hybridizes to sequence B′ present in a complementary polynucleotide comprising a complement of the target sequence via sequence complementarity between sequence B and B′; and (d) a polymerase that extends the first primer and the second primer to produce amplified polynucleotides; wherein the distance between the 5′ end of sequence A and the 3′ end of sequence B of the target sequence is 75nt or less.

In a related aspect, the disclosure provides a method of amplifying in a reaction mixture a plurality of different circular polynucleotides comprising a target sequence, wherein the target sequence comprises sequence A and sequence B oriented in a 5′ to 3′ direction. In some embodiments, the method comprises subjecting the reaction mixture to a nucleic acid amplification reaction, wherein the reaction mixture comprises: (a) the plurality of circular polynucleotides, wherein individual circular polynucleotides in the plurality comprise different junctions formed by circularizing individual polynucleotides having a 5′ end and a 3′ end; (b) a first primer comprising sequence A′, wherein the first primer specifically hybridizes to sequence A of the target sequence via sequence complementarity between sequence A and sequence A′; (c) a second primer comprising sequence B, wherein the second primer specifically hybridizes to sequence B′ present in a complementary polynucleotide comprising a complement of the target sequence via sequence complementarity between sequence B and B′; and (d) a polymerase that extends the first primer and the second primer to produce amplified polynucleotides; wherein sequence A and sequence B are endogenous sequences, and the distance between the 5′ end of sequence A and the 3′ end of sequence B of the target sequence is 75nt or less.

Whether amplifying circular polynucleotides or concatemers, such polynucleotides may be from any suitable sample sources (either directly, or indirectly, such as by amplification). A variety of suitable sample sources, optional extraction processes, types of polynucleotides, and types of sequence variants are described herein, such as with respect to any of the various aspects of the disclosure. Circular polynucleotides may be derived from circularizing non-circular polynucleotides. Non-limiting examples of circularization processes (e.g. with and without adapter oligonucleotides), reagents (e.g. types of adapters, use of ligases), reaction conditions (e.g. favoring self-joining), optional additional processing (e.g. post-reaction purification), and the junctions formed thereby are provided herein, such as with regard to any of the various aspects of the disclosure. Concatemers may be derived from amplification of circular polynucleotides. A variety of methods of amplifying polynucleotides (e.g. DNA and/or RNA) are available, non-limiting examples of which have also been described herein. In some embodiments, concatemers are generated by rolling circle amplification of circular polynucleotides.

In an example arrangement of the first and second primer with respect to a target sequence in the context of a single repeat (which will typically not be amplified unless circular) with concatemers comprising multiple copies of the target sequence. As noted with regard to other aspects described herein, this arrangement of primers may be referred to as “back to back” (B2B) or “inverted” primers. Amplification with B2B primers facilitates enrichment of circular and/or concatemeric templates. Moreover, this orientation combined with a relatively smaller footprint (total distance spanned by a pair of primers) permits amplification of a wider variety of fragmentation events around a target sequence, as a junction is less likely to occur between primers than in the arrangement of primers found in a typical amplification reaction (facing one another, spanning a target sequence). In some embodiments, the distance between the 5′ end of sequence A and the 3′ end of sequence B is about or less than about 200, 150, 100, 75, 50, 40, 30, 25, 20, 15, or fewer nucleotides. In some embodiments, sequence A is the complement of sequence B. In some embodiments, multiple pairs of B2B primers directed to a plurality of different target sequences are used in the same reaction to amplify a plurality of different target sequences in parallel (e.g. about or at least about 10, 50, 100, 150, 200, 250, 300, 400, 500, 1000, 2500, 5000, 10000, 15000, or more different target sequences). Primers can be of any suitable length, such as described elsewhere herein. Amplification may comprise any suitable amplification reaction under appropriate conditions, such as an amplification reaction described herein. In some embodiments, amplification is a polymerase chain reaction.

In some embodiments, B2B primers comprise at least two sequence elements, a first element that hybridizes to a target sequence via sequence complementarity, and a 5′ “tail” that does not hybridize to the target sequence during a first amplification phase at a first hybridization temperature during which the first element hybridizes (e.g. due to lack of sequence complementarity between the tail and the portion of the target sequence immediately 3′ with respect to where the first element binds). For example, the first primer comprises sequence C 5′ with respect to sequence A′, the second primer comprises sequence D 5′ with respect to sequence B, and neither sequence C nor sequence D hybridize to the plurality of concatemers (or circular polynucleotides) during a first amplification phase at a first hybridization temperature. In some embodiments in which such tailed primers are used, amplification can comprise a first phase and a second phase; the first phase comprises a hybridization step at a first temperature, during which the first and second primers hybridize to the concatemers (or circular polynucleotides) and primer extension; and the second phase comprises a hybridization step at a second temperature that is higher than the first temperature, during which the first and second primers hybridize to amplification products comprising extended first or second primers, or complements thereof, and primer extension. The number of amplification cycles at each of the two temperatures can be adjusted based on the products desired. Typically, the first temperature will be used for a relatively low number of cycles, such as about or less than about 15, 10, 9, 8, 7, 6, 5, or fewer cycles. The number of cycles at the higher temperature can be selected independently of the number of cycles at the first temperature, but will typically be as many or more cycles, such as about or at least about 5, 6, 7, 8, 9, 10, 15, 20, 25, or more cycles. The higher temperature favors hybridization between the first element and tail element of the primer in primer extension products over shorter fragments formed by hybridization between only the first element in a primer and an internal target sequence within a concatemer. Accordingly, the two-phase amplification may be used to reduce the extent to which short amplification products might otherwise be favored, thereby maintaining a relatively higher proportion of amplification products having two or more copies of a target sequence. For example, after 5 cycles (e.g. at least 5, 6, 7, 8, 9, 10, 15, 20, or more cycles) of hybridization at the second temperature and primer extension, at least 5% (e.g. at least 5%, 6%, 7%, 8%, 9%, 10%, 15%, 20%, 25%, 30%, or more) of amplified polynucleotides in the reaction mixture comprise two or more copies of the target sequence.

In some embodiments, amplification is under conditions that are skewed to increase the length of amplicons from concatemers. For example, the primer concentration can be lowered, such that not every priming site will hybridize a primer, thus making the PCR products longer. Similarly, decreasing the primer hybridization time during the cycles will similarly allow fewer primers to hybridize, thus also making the average PCR amplicon size increase. Furthermore, increasing the temperature and/or extension time of the cycles may similarly increase the average length of the PCR amplicons. Any combination of these techniques can be used.

In some embodiments, particularly where an amplification with B2B primers has been performed, amplification products are treated to filter the resulting amplicons on the basis of size to reduce and/or eliminate the number of monomers a mixture comprising concatemers. This can be done using a variety of available techniques, including, but not limited to, fragment excision from gels and gel filtration (e.g. to enrich for fragments larger than about 300, 400, 500, or more nucleotides in length); as well as SPRI beads (Agencourt AMPure XP) for size selection by fine-tuning the binding buffer concentration. For example, the use of 0.6× binding buffer during mixing with DNA fragments may be used to preferentially bind DNA fragments larger than about 500 base pairs (bp).

In some embodiments, the first primer comprises sequence C 5′ with respect to sequence A′, the second primer comprises sequence D 5′ with respect to sequence B, and neither sequence C nor sequence D hybridize to the plurality of circular polynucleotides during a first amplification phase at a first hybridization temperature. Amplification may comprise a first phase and a second phase; wherein the first phase comprises a hybridization step at a first temperature, during which the first and second primer hybridize to the circular polynucleotides or amplification products thereof prior to primer extension; and the second phase comprises a hybridization step at a second temperature that is higher than the first temperature, during which the first and second primers hybridize to amplification products comprising extended first or second primers or complements thereof. For example, the first temperature may be selected as about or more than about the Tm of sequence A′, sequence B, or the average of these, or a temperature that is greater than 1° C., 2° C., 3° C., 4° C., 5° C., 6° C., 7° C., 8° C., 9° C., 10° C., or higher than one of these Tm's. In this example, the second temperature may be selected to be about or more than about the Tm of the combined sequence (A′+C), the combine sequence (B+D), or the average of these, or a temperature that is greater than 1° C., 2° C., 3° C., 4° C., 5° C., 6° C., 7° C., 8° C., 9° C., 10° C., or higher than one of these Tm's. The term “Tm” is also referred to as the “melting temperature,” and generally represents the temperature at which 50% of an oligonucleotide consisting of a reference sequence (which may in fact be a sub-sequence within a larger polynucleotide) and its complementary sequence are hybridized (or separated). In general, Tm increases with increasing length, and as such, the Tm of sequence A′ is expected to be lower than the Tm of combination sequence (A′+C).

In one aspect, the disclosure provides a system for detecting a sequence variant. In some embodiments, the system comprises (a) a computer configured to receive a user request to perform a detection reaction on a sample; (b) an amplification system that performs a nucleic acid amplification reaction on the sample or a portion thereof in response to the user request, wherein the amplification reaction comprises the steps of (i) circularizing individual polynucleotides in a plurality of polynucleotides to form a plurality of circular polynucleotides using a ligase enzyme, each polynucleotide of the plurality having a junction between the 5′ end and 3′ end prior to ligation; (ii) degrading the ligase enzyme; and (ii) amplifying the circular polynucleotides after degrading the ligase enzyme to produce amplified polynucleotides; wherein polynucleotides are not purified or isolated between steps (i) and (iii); (c) a sequencing system that generates sequencing reads for polynucleotides amplified by the amplification system, identifies sequence differences between sequencing reads and a reference sequence, and calls a sequence difference that occurs in at least two circular polynucleotides having different junctions as the sequence variant; and (d) a report generator that sends a report to a recipient, wherein the report contains results for detection of the sequence variant. In some embodiments, the recipient is the user.

A computer for use in the system can comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules, and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc. A client-server, relational database architecture can be used in embodiments of the system. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments, the server computer handles all of the database functionality. The client computer can have software that handles all the front-end data management and can also receive data input from users.

The system can be configured to receive a user request to perform a detection reaction on a sample. The user request may be direct or indirect. Examples of direct request include those transmitted by way of an input device, such as a keyboard, mouse, or touch screen. Examples of indirect requests include transmission via a communication medium, such as over the internet (either wired or wireless).

The system can further comprise an amplification system that performs a nucleic acid amplification reaction on the sample or a portion thereof in response to the user request. A variety of methods of amplifying polynucleotides (e.g. DNA and/or RNA) are available. Amplification may be linear, exponential, or involve both linear and exponential phases in a multi-phase amplification process. Amplification methods may involve changes in temperature, such as a heat denaturation step, or may be isothermal processes that do not require heat denaturation. Non-limiting examples of suitable amplification processes are described herein, such as with regard to any of the various aspects of the disclosure. In some embodiments, amplification comprises rolling circle amplification (RCA). A variety of systems for amplifying polynucleotides are available, and may vary based on the type of amplification reaction to be performed. For example, for amplification methods that comprise cycles of temperature changes, the amplification system may comprise a thermocycler. An amplification system can comprise a real-time amplification and detection instrument, such as systems manufactured by Applied Biosystems, Roche, and Strategene. In some embodiments, the amplification reaction comprises the steps of (i) circularizing individual polynucleotides to form a plurality of circular polynucleotides, each of which having a junction between the 5′ end and 3′ end; and (ii) amplifying the circular polynucleotides. Samples, polynucleotides, primers, polymerases, and other reagents can be any of those described herein, such as with regard to any of the various aspects. Non-limiting examples of circularization processes (e.g. with and without adapter oligonucleotides), reagents (e.g. types of adapters, use of ligases), reaction conditions (e.g. favoring self-joining), optional additional processing (e.g. post-reaction purification), and the junctions formed thereby are provided herein, such as with regard to any of the various aspects of the disclosure. Systems can be selected and or designed to execute any such methods.

Systems may further comprise a sequencing system that generates sequencing reads for polynucleotides amplified by the amplification system, identifies sequence differences between sequencing reads and a reference sequence, and calls a sequence difference that occurs in at least two circular polynucleotides having different junctions as the sequence variant. The sequencing system and the amplification system may be the same, or comprise overlapping equipment. For example, both the amplification system and sequencing system may utilize the same thermocycler. A variety of sequencing platforms for use in the system are available, and may be selected based on the selected sequencing method. Examples of sequencing methods are described herein. Amplification and sequencing may involve the use of liquid handlers. Several commercially available liquid handling systems can be utilized to run the automation of these processes (see for example liquid handlers from Perkin-Elmer, Beckman Coulter, Caliper Life Sciences, Tecan, Eppendorf, Apricot Design, Velocity 11 as examples). A variety of automated sequencing machines are commercially available, and include sequencers manufactured by Life Technologies (SOLiD platform, and pH-based detection), Roche (454 platform), Illumina (e.g. flow cell based systems, such as Genome Analyzer devices). Transfer between 2, 3, 4, 5, or more automated devices (e.g. between one or more of a liquid handler and a sequencing device) may be manual or automated.

Methods for identifying sequence differences and calling sequence variants with respect to a reference sequence are described herein, such as with regard to any of the various aspects of the disclosure. The sequencing system will typically comprise software for performing these steps in response to an input of sequencing data and input of desired parameters (e.g. selection of a reference genome). Examples of alignment algorithms and aligners implementing these algorithms are described herein, including but not limited to the Needleman-Wunsch algorithm (see e.g. the EMBOSS Needle aligner available at www.ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa emboss_water/nucleotide.html, optionally with default settings). Optimal alignment may be assessed using any suitable parameters of a chosen algorithm, including default parameters. Such alignment algorithms may form part of the sequencing system.

The system can further comprise a report generator that sends a report to a recipient, wherein the report contains results for detection of the sequence variant. A report may be generated in real-time, such as during a sequencing read or while sequencing data is being analyzed, with periodic updates as the process progresses. In addition, or alternatively, a report may be generated at the conclusion of the analysis. The report may be generated automatically, such when the sequencing system completes the step of calling all sequence variants. In some embodiments, the report is generated in response to instructions from a user. In addition to the results of detection of the sequence variant, a report may also contain an analysis based on the one or more sequence variants. For example, where one or more sequence variants are associated with a particular contaminant or phenotype, the report may include information concerning this association, such as a likelihood that the contaminant or phenotype is present, at what level, and optionally a suggestion based on this information (e.g. additional tests, monitoring, or remedial measures). The report can take any of a variety of forms. It is envisioned that data relating to the present disclosure can be transmitted over such networks or connections (or any other suitable means for transmitting information, including but not limited to mailing a physical report, such as a print-out) for reception and/or for review by a receiver. The receiver can be but is not limited to an individual, or electronic system (e.g. one or more computers, and/or one or more servers).

In one aspect, the disclosure provides a computer-readable medium comprising codes that, upon execution by one or more processors, implement a method of detecting a sequence variant. In some embodiments, the implemented method comprises: (a) receiving a customer request to perform a detection reaction on a sample; (b) performing a nucleic acid amplification reaction on the sample or a portion thereof in response to the customer request, wherein the amplification reaction comprises the steps of (i) circularizing individual polynucleotides in a plurality of polynucleotides to form a plurality of circular polynucleotides using a ligase enzyme, wherein each polynucleotide of the plurality of polynucleotides has a 5′ end and 3′ end prior to ligation; (ii) degrading the ligase enzyme; and (ii) amplifying the circular polynucleotides after degrading the ligase enzyme to produce amplified polynucleotides; wherein polynucleotides are not purified or isolated between steps (i) and (iii); (c) performing a sequencing analysis comprising the steps of (i) generating sequencing reads for polynucleotides amplified in the amplification reaction; (ii) identifying sequence differences between sequencing reads and a reference sequence; and (iii) calling a sequence difference that occurs in at least two circular polynucleotides having different junctions as the sequence variant; and (d) generating a report that contains results for detection of the sequence variant.

A machine readable medium comprising computer-executable code may take many forms, including but not limited to, a tangible storage medium, a carrier wave medium or physical transmission medium. Non-volatile storage media include, for example, optical or magnetic disks, such as any of the storage devices in any computers) or the like, such as may be used to implement the databases, etc. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device comprising a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

In some embodiments of any of the various aspects disclosed herein, the methods, compositions, and systems have therapeutic applications, such as in the characterization of a patient sample and optionally diagnosis of a condition of a subject. Therapeutic applications may also include informing the selection of therapies to which a patient may be most responsive (also referred to as “theranostics”), and actual treatment of a subject in need thereof, based on the results of a method described herein. In particular, methods and compositions disclosed herein may be used to diagnose tumor presence, progression and/or metastasis of tumors, especially when the polynucleotides analyzed comprise or consist of cfDNA, ctDNA, cfRNA, or fragmented tumor DNA. In some embodiments, a subject is monitored for treatment efficacy. For example, by monitoring ctDNA over time, a decrease in ctDNA can be used as an indication of efficacious treatment, while increases can facilitate selection of different treatments or different dosages. Other uses include evaluations of organ rejection in transplant recipients (where increases in the amount of circulating DNA corresponding to the transplant donor genome is used as an early indicator of transplant rejection), and genotyping/isotyping of pathogen infections, such as viral or bacterial infections. Detection of sequence variants in circulating fetal DNA may be used to diagnose a condition of a fetus.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. Typically, prophylactic benefit includes reducing the incidence and/or worsening of one or more diseases, conditions, or symptoms under treatment (e.g. as between treated and untreated populations, or between treated and untreated states of a subject). Improving a treatment outcome may include diagnosing a condition of a subject in order to identify the subject as one that will or will not benefit from treatment with one or more therapeutic agents, or other therapeutic intervention (such as surgery). In such diagnostic applications, the overall rate of successful treatment with the one or more therapeutic agents may be improved, relative to its effectiveness among patients grouped without diagnosis according to a method of the present disclosure (e.g. an improvement in a measure of therapeutic efficacy by at least about 10%, 20%, 30%, 40%, 50%, 60%, 70%, 80%, 90%, 95%, or more).

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells, and their progeny of a biological entity obtained in vivo or cultured in vitro are also encompassed.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder, or pathological condition.

In some embodiments of the various methods described herein, the sample is from a subject. A subject can be any organism, non-limiting examples of which include plants, animals, fungi, protists, monerans, viruses, mitochondria, and chloroplasts. Sample polynucleotides can be isolated from a subject, such as a cell sample, tissue sample, bodily fluid sample, or organ sample (or cell cultures derived from any of these), including, for example, cultured cell lines, biopsy, blood sample, cheek swab, or fluid sample containing a cell (e.g. saliva). In some cases, the sample does not comprise intact cells, is treated to remove cells, or polynucleotides are isolated without a cellular extractions step (e.g. to isolate cell-free polynucleotides, such as cell-free DNA). Other examples of sample sources include those from blood, urine, feces, nares, the lungs, the gut, other bodily fluids or excretions, materials derived therefrom, or combinations thereof. The subject may be an animal, including but not limited to, a cow, a pig, a mouse, a rat, a chicken, a cat, a dog, etc., and is usually a mammal, such as a human. In some embodiments, the sample comprises tumor cells, such as in a sample of tumor tissue from a subject. In some embodiments, the sample is a blood sample or a portion thereof (e.g. blood plasma or serum). Serum and plasma may be of particular interest, due to the relative enrichment for tumor DNA associated with the higher rate of malignant cell death among such tissues. A sample may be a fresh sample, or a sample subjected to one or more storage processes (e.g. paraffin-embedded samples, particularly formalin-fixed paraffin-embedded (FFPE) sample). In some embodiments, a sample from a single individual is divided into multiple separate samples (e.g. 2, 3, 4, 5, 6, 7, 8, 9, 10, or more separate samples) that are subjected to methods of the disclosure independently, such as analysis in duplicate, triplicate, quadruplicate, or more. Where a sample is from a subject, the reference sequence may also be derived from the subject, such as a consensus sequence from the sample under analysis or the sequence of polynucleotides from another sample or tissue of the same subject. For example, a blood sample may be analyzed for ctDNA mutations, while cellular DNA from another sample (e.g. buccal or skin sample) is analyzed to determine the reference sequence.

Polynucleotides may be extracted from a sample, with or without extraction from cells in a sample, according to any suitable method. A variety of kits are available for extraction of polynucleotides, selection of which may depend on the type of sample, or the type of nucleic acid to be isolated. Examples of extraction methods are provided herein, such as those described with respect to any of the various aspects disclosed herein. In one example, the sample may be a blood sample, such as a sample collected in an EDTA tube (e.g. BD Vacutainer). Plasma can be separated from the peripheral blood cells by centrifugation (e.g. 10 minutes at 1900×g at 4° C.). Plasma separation performed in this way on a 6 mL blood sample will typically yield 2.5 to 3 mL of plasma. Circulating cell-free DNA can be extracted from a plasma sample, such as by using a QIAmp Circulating Nucleic Acid Kit (Qiagene), according the manufacturer's protocol. DNA may then be quantified (e.g. on an Agilent 2100 Bioanalyzer with High Sensitivity DNA kit (Agilent)). As an example, yield of circulating DNA from such a plasma sample from a healthy person may range from 1 ng to 10 ng per mL of plasma, with significantly more in cancer patient samples.

Polynucleotides can also be derived from stored samples, such frozen or archived samples. One common method for storing samples is to formalin-fix and paraffin-embed them. However, this process is also associated with degradation of nucleic acids. Polynucleotides processed and analyzed from an FFPE sample may include short polynucleotides, such as fragments in the range of 50-200 base pairs, or shorter. A number of techniques exist for the purification of nucleic acids from fixed paraffin-embedded samples, such as those described in WO2007133703, and methods described by Foss, et al Diagnostic Molecular Pathology, (1994) 3:148-155 and Paska, C., et al Diagnostic Molecular Pathology, (2004) 13:234-240. Commercially available kits may be used for purifying polynucleotides from FFPE samples, such as Ambion's Recoverall Total Nucleic acid Isolation kit. Typical methods start with a step that removes the paraffin from the tissue via extraction with Xylene or other organic solvent, followed by treatment with heat and a protease like proteinase K which cleaves the tissue and proteins and helps to release the genomic material from the tissue. The released nucleic acids can then be captured on a membrane or precipitated from solution, washed to removed impurities and for the case of mRNA isolation, a DNase treatment step is sometimes added to degrade unwanted DNA. Other methods for extracting FFPE DNA are available and can be used in the methods of the present disclosure.

In some embodiments, the plurality of polynucleotides comprise cell-free polynucleotides, such as cell-free DNA (cfDNA), cell-free RNA (cfRNA), circulating tumor DNA (ctDNA), or circulating tumor RNA (ctRNA). Cell-free DNA circulates in both healthy and diseased individuals. Cell-free RNA circulates in both healthy and diseased individuals. cfDNA from tumors (ctDNA) is not confined to any specific cancer type, but appears to be a common finding across different malignancies. According to some measurements, the free circulating DNA concentration in plasma is about 14-18 ng/ml in control subjects and about 180-318 ng/ml in patients with neoplasias. Apoptotic and necrotic cell death contribute to cell-free circulating DNA in bodily fluids. For example, significantly increased circulating DNA levels have been observed in plasma of prostate cancer patients and other prostate diseases, such as Benign Prostate Hyperplasia and Prostatits. In addition, circulating tumor DNA is present in fluids originating from the organs where the primary tumor occurs. Thus, breast cancer detection can be achieved in ductal lavages; colorectal cancer detection in stool; lung cancer detection in sputum, and prostate cancer detection in urine or ejaculate. Cell-free DNA may be obtained from a variety of sources. One common source is blood samples of a subject. However, cfDNA or other fragmented DNA may be derived from a variety of other sources. For example, urine and stool samples can be a source of cfDNA, including ctDNA. Cell-free RNA may be obtained from a variety of sources.

In some embodiments, methods herein are used in detection of minimal residual disease (MRD). In some embodiments, detecting MRD comprises sequencing a tumor sample from a subject to identify one or more tumor specific variants compared with a healthy sample from the subject. In some cases, specific variants are sequenced in a sample from the subject after treatment, such as a sample from the subject comprising cfDNA. In some cases, identification of the one or more tumor specific variant in sequence obtained from the sample from the subject (e.g., cfDNA from the subject) after treatment indicates that MRD is present in the subject. In some cases, when the one or more tumor specific variant is not identified in sequence obtained from the sample from the subject (e.g., cfDNA from the subject) after treatment indicates that MRD is not present in the subject. In some cases, when MRD is observed in the subject, the subject is given additional treatment for the cancer.

In some embodiments, polynucleotides are subjected to subsequent steps (e.g. circularization and amplification) without an extraction step, and/or without a purification step. For example, a fluid sample may be treated to remove cells without an extraction step to produce a purified liquid sample and a cell sample, followed by isolation of DNA from the purified fluid sample. A variety of procedures for isolation of polynucleotides are available, such as by precipitation or non-specific binding to a substrate followed by washing the substrate to release bound polynucleotides. Where polynucleotides are isolated from a sample without a cellular extraction step, polynucleotides will largely be extracellular or “cell-free” polynucleotides. For example, cell-free polynucleotides may include cell-free DNA (also called “circulating” DNA). In some embodiments, the circulating DNA is circulating tumor DNA (ctDNA) from tumor cells, such as from a body fluid or excretion (e.g. blood sample). Cell-free polynucleotides may include cell-free RNA (also called “circulating” RNA). In some embodiments, the circulating RNA is circulating tumor RNA (ctRNA) from tumor cells. Tumors frequently show apoptosis or necrosis, such that tumor nucleic acids are released into the body, including the blood stream of a subject, through a variety of mechanisms, in different forms and at different levels. Typically, the size of the ctDNA can range between higher concentrations of smaller fragments, generally 70 to 200 nucleotides in length, to lower concentrations of large fragments of up to thousands kilobases.

In some embodiments of any of the various aspects described herein, detecting a sequence variant comprises detecting mutations (e.g. rare somatic mutations) with respect to a reference sequence or in a background of no mutations, where the sequence variant is correlated with disease. In general, sequence variants for which there is statistical, biological, and/or functional evidence of association with a disease or trait are referred to as “causal genetic variants.” A single causal genetic variant can be associated with more than one disease or trait. In some embodiments, a causal genetic variant can be associated with a Mendelian trait, a non-Mendelian trait, or both. Causal genetic variants can manifest as variations in a polynucleotide, such 1, 2, 3, 4, 5, 6, 7, 8, 9, 10, 20, 50, or more sequence differences (such as between a polynucleotide comprising the causal genetic variant and a polynucleotide lacking the causal genetic variant at the same relative genomic position). Non-limiting examples of types of causal genetic variants include single nucleotide polymorphisms (SNP), deletion/insertion polymorphisms (DIP), copy number variants (CNV), short tandem repeats (STR), restriction fragment length polymorphisms (RFLP), simple sequence repeats (SSR), variable number of tandem repeats (VNTR), randomly amplified polymorphic DNA (RAPD), amplified fragment length polymorphisms (AFLP), inter-retrotransposon amplified polymorphisms (IRAP), long and short interspersed elements (LINE/SINE), long tandem repeats (LTR), mobile elements, retrotransposon microsatellite amplified polymorphisms, retrotransposon-based insertion polymorphisms, sequence specific amplified polymorphism, and heritable epigenetic modification (for example, DNA methylation). A causal genetic variant may also be a set of closely related causal genetic variants. Some causal genetic variants may exert influence as sequence variations in RNA polynucleotides. At this level, some causal genetic variants are also indicated by the presence or absence of a species of RNA polynucleotides. Also, some causal genetic variants result in sequence variations in protein polypeptides. A number of causal genetic variants have been reported. An example of a causal genetic variant that is a SNP is the Hb S variant of hemoglobin that causes sickle cell anemia. An example of a causal genetic variant that is a DIP is the delta508 mutation of the CFTR gene which causes cystic fibrosis. An example of a causal genetic variant that is a CNV is trisomy 21, which causes Down's syndrome. An example of a causal genetic variant that is an STR is tandem repeat that causes Huntington's disease. Non-limiting examples of causal genetic variants and diseases with which they are associated are provided in Table 1. Additional non-limiting examples of causal genetic variants are described in WO2014015084. Further examples of genes in which mutations are associated with diseases, and in which sequence variants may be detected according to a method of the disclosure, are provided in Table 2.

TABLE 1

| Disease | Gene | Variant Name |
|---|---|---|
| 21-Hydroxylase Deficiency | CYP21A2 | F306+t |
| 21-Hydroxylase Deficiency | CYP21A2 | F306+t |
| 21-Hydroxylase Deficiency | CYP21A3 | g.655A/C > G |
| 21-Hydroxylase Deficiency | CYP21A4 | g.655A/C > G |
| 21-Hydroxylase Deficiency | CYP21A6 | G110del8nt |
| 21-Hydroxylase Deficiency | CYP21A5 | G110del8nt |
| 21-Hydroxylase Deficiency | CYP21A7 | I172N, rs34607927 |
| 21-Hydroxylase Deficiency | CYP21A2 | I236N |
| 21-Hydroxylase Deficiency | CYP21A2 | M239K, rs6476 |
| 21-Hydroxylase Deficiency | CYP21A2 | P30L |
| 21-Hydroxylase Deficiency | CYP21A2 | P453S |
| 21-Hydroxylase Deficiency | CYP21A2 | Q318X |
| 21-Hydroxylase Deficiency | CYP21A2 | R356W |
| 21-Hydroxylase Deficiency | CYP21A2 | V237E, rs12530380 |
| 21-Hydroxylase Deficiency | CYP21A2 | V281L, rs6471 |
| ABCC8-Related Hyperinsulinism | ABCC8 | 3992-9G > A |
| ABCC8-Related Hyperinsulinism | ABCC8 | delF1388 |
| ABCC8-Related Hyperinsulinism | ABCC8 | delF1388 |
| ABCC8-Related Hyperinsulinism | ABCC8 | V187D |
| Achondroplasia | FGFR3 | G375C |
| Achondroplasia | FGFR3 | G380R, rs28931614 |
| Achromatopsia | CNGB3 | c.1148delC |
| Achromatopsia | CNGB3 | c.1148delC |
| Achromatopsia | CNGB3 | c.819-826del8 |
| Achromatopsia | CNGB3 | c.819-826del8 |
| Achromatopsia | CNGB3 | c.886-896del11insT |
| Achromatopsia | CNGB3 | c.886-896del11insT |
| Achromatopsia | CNGB3 | c.991-3T > G |
| Achromatopsia | CNGB3 | p.Arg403Gln |
| Achromatopsia | CNGB3 | p.Glu336X |
| Adenosine Monophosphate Deaminase | AMPD1 | P48L |
| 1 | | |
| Adenosine Monophosphate Deaminase | AMPD1 | Q12X, rs17602729 |
| 1 | | |
| Agenesis of Corpus Callosum with | SLC12A6 | c.2436delG |
| Neuronopathy | | |
| Agenesis of Corpus Callosum with | SLC12A6 | c.2436delG |
| Neuronopathy | | |
| Alkaptonuria | HGD | c.174delA |
| Alkaptonuria | HGD | c.174delA |
| Alkaptonuria | HGD | c.457_458insG |
| Alkaptonuria | HGD | c.457_458insG |
| Alkaptonuria | HGD | G161R |
| Alkaptonuria | HGD | G270R |
| Alkaptonuria | HGD | IVS1−1G > A |
| Alkaptonuria | HGD | IVS5+1G > A |
| Alkaptonuria | HGD | Met368Val |
| Alkaptonuria | HGD | P230S |
| Alkaptonuria | HGD | S47L |
| Alkaptonuria | HGD | V300G |
| Alpha-1-Antitrypsin Deficiency | SERPINA1 | Arg101His, rs709932 |
| Alpha-1-Antitrypsin Deficiency | SERPINA1 | Glu264Val |
| Alpha-1-Antitrypsin Deficiency | SERPINA1 | Glu342Lys, rs28929474 |
| Alpha-1-Antitrypsin Deficiency | SERPINA1 | Glu376Asp, rs1303 |
| Alpha-Mannosidosis | MAN2B1 | IVS14+1G > C |
| Alpha-Mannosidosis | MAN2B1 | p.L809P |
| Alpha-Mannosidosis | MAN2B1 | p.R750W |
| Alpha-Sarcoglycanopathy | SGCA | R77C, rs28933693 |
| Alpha-Thalassemia | HBA2 | H19D |
| Alpha-Thalassemia | HBA1 | HbQ |
| Alpha-Thalassemia | HBA1, HBA2 | 3.7 kb (type I) deletion alpha-2 |
| Alpha-Thalassemia | HBA1, HBA2 | 3.7 kb (type I) deletion alpha-2 |
| Alpha-Thalassemia | HBA1, HBA2 | HbVar database id # 1086, -- |
| | | (SEA); deletion of ~20 kb |
| | | including both alpha-globin |
| | | genes alpha-Thal-1 |
| Alpha-Thalassemia | HBA1, HBA2 | HbVar database id # 1086, -- |
| | | (SEA); deletion of ~20 kb |
| | | including both alpha-globin |
| | | genes alpha-Thal-1 |
| Alpha-Thalassemia | HBA1, HBA2 | HbVar database id # 1087, -- |
| | | (MED-I); deletion of ~17.5 kb |
| | | including both alpha-globin |
| | | genes alpha-Thal-1 |
| Alpha-Thalassemia | HBA1, HBA2 | HbVar database id # 1087, -- |
| | | (MED-I); deletion of ~17.5 kb |
| | | including both alpha-globin |
| | | genes alpha-Thal-1 |
| Alpha-Thalassemia | HBA1, HBA2 | HbVar database id # 1088, - |
| | | (alpha)20.5; this 20.5 kb |
| | | deletion involves alpha2 and |
| | | the 5′ end of alphal; alpha- |
| | | Thal-1 |
| Alpha-Thalassemia | HBA1, HBA2 | HbVar database id # 1088, - |
| | | (alpha)20.5; this 20.5 kb |
| | | deletion involves alpha2 and |
| | | the 5′ end of alphal; alpha- |
| | | Thal-1 |
| Alpha-Thalassemia | HBA1, HBA2 | HbVar database id # 1094, -- |
| | | (FIL); a deletion of 30-34 kb |
| | | involving the alphal, alpha2, |
| | | and zeta genes alpha-Thal-1 |
| Alpha-Thalassemia | HBA1, HBA2 | HbVar database id # 1094, -- |
| | | (FIL); a deletion of 30-34 kb |
| | | involving the alphal, alpha2, |
| | | and zeta genes alpha-Thal-1 |
| Alpha-Thalassemia | HBA1, HBA2 | HbVar database id # 1095, -- |
| | | (THAI); a deletion of 34-38 kb |
| | | involving the alphal, alpha2, |
| | | and zeta genes alpha-Thal-1 |
| Alpha-Thalassemia | HBA1, HBA2 | HbVar database id # 1095, -- |
| | | (THAI); a deletion of 34-38 kb |
| | | involving the alphal, alpha2, |
| | | and zeta genes alpha-Thal-1 |
| Alpha-Thalassemia | HBA1, HBA2 | HbVar database id # 1097, -- |
| | | (MED-II); a deletion of 26.5 kb |
| | | involving the two alpha and |
| | | zeta genes alpha-Thal-1 |
| Alpha-Thalassemia | HBA1, HBA2 | HbVar database id # 1097, -- |
| | | (MED-II); a deletion of 26.5 kb |
| | | involving the two alpha and |
| | | zeta genes alpha-Thal-1 |
| Alpha-Thalassemia | HBA2 | HbVar database id # 187 |
| Alpha-Thalassemia | HBA2 | HbVar database id # 2598, IVS |
| | | I-5 (G > A) |
| Alpha-Thalassemia | HBA1, HBA2 | HbVar database id # 703 |
| Alpha-Thalassemia | HBA1, HBA2 | HbVar database id # 704 |
| Alpha-Thalassemia | HBA1, HBA2 | HbVar database id # 705, Hb |
| | | Koya Dora |
| Alpha-Thalassemia | HBA1, HBA2 | HbVar database id # 707, |
| | | rs41412046 |
| Alpha-Thal assemia | HBA1 | HbVar database id # 87 |
| Alpha-Thalassemia | HBA1, HBA2 | HbVar database id # 969, Poly |
| | | A (A->G); AATAAA-> |
| | | AATGAA beta+ |
| Alpha-Thalassemia | HBA1, HBA2 | HbVar database id # 971, Poly |
| | | A (A->G); AATAAA-> |
| | | AATAAG beta+ |
| Alpha-Thal assemia | HBA2 | MIT |
| Alpha-Thalassemia | HBA1 | W14X |
| Angiotensin II Receptor, Type 1 | AGTR1 | A1166C |
| Apolipoprotein E Genotyping | APOE | p.C112R, rs429358 |
| Apolipoprotein E Genotyping | APOE | p.R158C, rs7412 |
| Argininosuccinicaciduria | ASL | R385C |
| ARSACS | SACS | 5254C > T |
| ARSACS | SACS | 6594delT |
| ARSACS | SACS | 6594delT |
| Aspartylglycosaminuria | AGA | c.199_200delGA |
| Aspartylglycosaminuria | AGA | c.199_200delGA |
| Aspartylglycosaminuria | AGA | C163S |
| Ataxia with Vitamin E Deficiency | TTPA | 744delA |
| Ataxia with Vitamin E Deficiency | TTP A | 744delA |
| Ataxia-Telangiectasia | ATM | R35X |
| Autoimmune Polyendocrinopathy | AIRE | c.1163_1164insA |
| Syndrome Type 1 | | |
| Autoimmune Polyendocrinopathy | AIRE | c.1163_1164insA |
| Syndrome Type 1 | | |
| Autoimmune Polyendocrinopathy | AIRE | c.769C > T |
| Syndrome Type 1 | | |
| Autoimmune Polyendocrinopathy | AIRE | c.967_979del |
| Syndrome Type 1 | | |
| Autoimmune Polyendocrinopathy | AIRE | c.967_979del |
| Syndrome Type 1 | | |
| Autoimmune Polyendocrinopathy | AIRE | Y85C |
| Syndrome Type 1 | | |
| Bardet-Biedl Syndrome | BBS1 | M390R |
| Bardet-Biedl Syndrome | BBS10 | p.C91LfsX4 |
| Bardet-Biedl Syndrome | BBS10 | p.C91LfsX4 |
| Best Vitelliform Macular Dystrophy | BEST1 | c.G383C |
| Beta-Sarcoglycanopathy | SGCB | S114F |
| Beta-Thalassemia | HBB | −28 (A->G) beta+ |
| Beta-Thalassemia | HBB | −29 (A->G) beta+ |
| Beta-Thalassemia | HBB | −29A > G |
| Beta-Thalassemia | HBB | −30 (T->A) beta+ |
| Beta-Thalassemia | HBB | −87 (C->G) beta+ |
| Beta-Thalassemia | HBB | −88C > T |
| Beta-Thalassemia | HBB | CAP+1 (A->C) beta+ |
| Beta-Thalassemia | HBB | Codon 15 (G->A); TGG(Trp)-> |
| | | TAG(stop codon) beta0, |
| | | rs34716011 |
| Beta-Thalassemia | HBB | Codon 15 (G->A); TGG(Trp)-> |
| | | TAG(stop codon) beta0, |
| | | rs34716011 |
| Beta-Thal assemia | HBB | Codon 16 (−C); GGC(Gly)-> |
| | | GG-beta0 |
| Beta-Thalassemia | HBB | Codon 16 (−C); GGC(Gly)-> |
| | | GG-beta0 |
| Beta-Thal assemia | HBB | Codon 17 (A->T); AAG(Lys)-> |
| | | TAG(stop codon) beta0 |
| Beta-Thalassemia | HBB | Codon 24 (T->A); GGT(Gly)-> |
| | | GGA(Gly) beta+ |
| Beta-Thalassemia | HBB | Codon 39 (C->T); CAG(Gln)-> |
| | | TAG(stop codon) beta0 |
| Beta-Thalassemia | HBB | Codon 5 (−CT); CCT(Pro)->C -- |
| | | beta0 |
| Beta-Thalassemia | HBB | Codon 5 (−CT); CCT(Pro)->C -- |
| | | beta0 |
| Beta-Thalassemia | HBB | Codon 6 (−A); GAG(Glu)->G- |
| | | G beta0 |
| Beta-Thalassemia | HBB | Codon 6 (−A); GAG(Glu)->G- |
| | | G beta0 |
| Beta-Thalassemia | HBB | Codon 8 (−AA); AAG(Lys)->-- |
| | | G beta0 |
| Beta-Thalassemia | HBB | Codon 8 (−AA); AAG(Lys)->-- |
| | | G beta0 |
| Beta-Thalassemia | HBB | Codons 41/42 (−TTCT); |
| | | TTCTTT(Phe-Phe)->---- TT |
| | | beta0 |
| Beta-Thalassemia | HBB | Codons 41/42 (−TTCT); |
| | | TTCTTT(Phe-Phe)->---- TT |
| | | beta0 |
| Beta-Thalassemia | HBB | Codons 71/72 (+A); TTT |
| | | AGT(Phe Ser)->TIT A AGT; |
| | | beta0 |
| Beta-Thalassemia | HBB | Codons 71/72 (+A); TTT |
| | | AGT(Phe Ser)->TTT A AGT; |
| | | beta0 |
| Beta-Thalassemia | HBB | Codons 8/9 (+G); AAG |
| | | TCT(Lys;Ser)->AAG G TCT |
| | | beta0 |
| Beta-Thalassemia | HBB | Codons 8/9 (+G); AAG |
| | | TCT(Lys;Ser)->AAG G TCT |
| | | beta0 |
| Beta-Thalassemia | HBB | HbVar database id # 889, IVS- |
| | | II-654 (C->T); |
| | | AAGGCAATA-> |
| | | AAG\hat{ }GTAATA |
| | | beta+(severe) |
| Beta-Thalassemia | HBB | HbVar database id # 890, IVS- |
| | | II-705 (T->G); |
| | | GATGTAAGA-> |
| | | GAG\hat{ }GTAAGA beta+ |
| Beta-Thalassemia | HBB | HbVar database id # 891, IVS- |
| | | II-745 (C->G); |
| | | CAGCTACCAT-> |
| | | CAG\hat{ }GTACCAT beta+ |
| Beta-Thalassemia | HBB | HbVar database id # 979, 619 |
| | | bp deletion beta0 |
| Beta-Thalassemia | HBB | 619 bp deletion beta0 |
| Beta-Thalassemia | HBB | IVS-I-1 (G->A); |
| | | AG\hat{ }GTTGGT->AGATTGGT |
| | | beta0 |
| Beta-Thalassemia | HBB | IVS-I-1 (G->T); |
| | | AG\hat{ }GTTGGT->AGTTTGGT |
| | | beta0 |
| Beta-Thalassemia | HBB | IVS-I-110 (G->A) beta+; the |
| | | mutation is 21 nucleotides 5′ to |
| | | the acceptor splice site AG\hat{ }GC |
| Beta-Thalassemia | HBB | IVS-I-5 (G->C) beta+(severe) |
| Beta-Thalassemia | HBB | IVS-II-1 (G->A); beta0 |
| Beta-Thalassemia | HBB | IVS-II-844 (C->G); beta+ |
| Beta-Thalassemia | HBB | IVS1+6T > C |
| Beta-Thalassemia | HBB | IVS11-849A > C |
| Beta-Thalassemia | HBB | IVS11-849A > G |
| Biotinidase Deficiency | BTD | A171T, rs13073139 |
| Biotinidase Deficiency | BTD | D252G, rs28934601 |
| Biotinidase Deficiency | BTD | D444H, rs13078881 |
| Biotinidase Deficiency | BTD | F403V |
| Biotinidase Deficiency | BTD | G98:d713 |
| Biotinidase Deficiency | BTD | G98:d713 |
| Biotinidase Deficiency | BTD | Q456H |
| Biotinidase Deficiency | BTD | R157H |
| Biotinidase Deficiency | BTD | R538C |
| Blau Syndrome | NOD2 | E383K |
| Blau Syndrome | NOD2 | L469F |
| Blau Syndrome | NOD2 | R334Q |
| Blau Syndrome | NOD2 | R334W |
| Bloom Syndrome | BLM | 2407insT |
| Bloom Syndrome | BLM | 2407insT |
| Bloom Syndrome | BLM | 736delATCTGAinsTAGATTC |
| | | (2281del6/ins7) |
| Bloom Syndrome | BLM | 736delATCTGAinsTAGATTC |
| | | (2281del6/ins7) |
| BRCA1 Hereditary Breast/Ovarian | BRCA1 | 185delAG |
| Cancer | | |
| BRCA1 Hereditary Breast/Ovarian | BRCA1 | 185delAG |
| Cancer | | |
| BRCA1 Hereditary Breast/Ovarian | BRCA1 | 5382insC |
| Cancer | | |
| BRCA1 Hereditary Breast/Ovarian | BRCA1 | 5382insC |
| Cancer | | |
| BRCA1 Hereditary Breast/Ovarian | BRCA1 | Tyr978X |
| Cancer | | |
| BRCA2 Hereditary Breast/Ovarian | BRCA2 | 6174delT |
| Cancer | | |
| BRCA2 Hereditary Breast/Ovarian | BRCA2 | 6174delT |
| Cancer | | |
| BRCA2 Hereditary Breast/Ovarian | BRCA2 | 8765delAG |
| Cancer | | |
| BRCA2 Hereditary Breast/Ovarian | BRCA2 | 8765delAG |
| Cancer | | |
| Canavan Disease | ASPA | A305E (914C > A), rs28940574 |
| Canavan Disease | ASPA | E285A (854A > C), rs28940279 |
| Canavan Disease | ASPA | IVS2-2A > G (433-2A > G) |
| Canavan Disease | ASPA | Y231X (693C > A) |
| Carnitine Palmitoyltransferase IA | CPTIA | G710E |
| Deficiency | | |
| Carnitine Palmitoyltransferase IA | CPTIA | P479L |
| Deficiency | | |
| Carnitine Palmitoyltransferase II | CPT2 | G549D |
| Deficiency | | |
| Carnitine Palmitoyltransferase II | CPT2 | L178F 534 ins/25 bp del |
| Deficiency | | |
| Carnitine Palmitoyltransferase II | CPT2 | L178F 534 ins/25 bp del |
| Deficiency | | |
| Carnitine Palmitoyltransferase II | CPT2 | P227L |
| Deficiency | | |
| Carnitine Palmitoyltransferase II | CPT2 | P50H |
| Deficiency | | |
| Carnitine Palmitoyltransferase II | CPT2 | P604S |
| Deficiency | | |
| Carnitine Palmitoyltransferase II | CPT2 | Q413fs |
| Deficiency | | |
| Carnitine Palmitoyltransferase II | CPT2 | Q413fs |
| Deficiency | | |
| Carnitine Palmitoyltransferase II | CPT2 | Q550R |
| Deficiency | | |
| Carnitine Palmitoyltransferase II | CPT2 | R124X |
| Deficiency | | |
| Carnitine Palmitoyltransferase II | CPT2 | R503C |
| Deficiency | | |
| Carnitine Palmitoyltransferase II | CPT2 | R631C |
| Deficiency | | |
| Carnitine Palmitoyltransferase II | CPT2 | S113L |
| Deficiency | | |
| Carnitine Palmitoyltransferase II | CPT2 | s38fs |
| Deficiency | | |
| Carnitine Palmitoyltransferase II | CPT2 | s38fs |
| Deficiency | | |
| Carnitine Palmitoyltransferase II | CPT2 | Y628S, rs28936673 |
| Deficiency | | |
| Cartilage-Hair Hypoplasia | RMRP | g.262G > T |
| Cartilage-Hair Hypoplasia | RMPR | g.70A > G |
| CFTR-Related Disorders | CFTR | 1811+1.6 kbA->G |
| CFTR-Related Disorders | CFTR | 2183AA > G |
| CFTR-Related Disorders | CFTR | 2183AA > G |
| CFTR-Related Disorders | CFTR | 3849+10 kbC > T |
| CFTR-Related Disorders | CFTR | A455E |
| CFTR-Related Disorders | CFTR | A559T |
| CFTR-Related Disorders | CFTR | C524X |
| CFTR-Related Disorders | CFTR | 574delA, 574delA |
| CFTR-Related Disorders | CFTR | 574delA, 574delA |
| CFTR-Related Disorders | CFTR | 2108delA, 2108delA |
| CFTR-Related Disorders | CFTR | 2108delA, 2108delA |
| CFTR-Related Disorders | CFTR | 3171delC, 3171delC |
| CFTR-Related Disorders | CFTR | 3171delC, 3171delC |
| CFTR-Related Disorders | CFTR | 621+1G->T |
| CFTR-Related Disorders | CFTR | 2105-2117del13insAGAAA |
| CFTR-Related Disorders | CFTR | 2105-2117del13insAGAAA |
| CFTR-Related Disorders | CFTR | 711+1G->T |
| CFTR-Related Disorders | CFTR | 711+5G->A |
| CFTR-Related Disorders | CFTR | 712−1G->T |
| CFTR-Related Disorders | CFTR | 1288insTA, 1288insTA |
| CFTR-Related Disorders | CFTR | 1288insTA, 1288insTA |
| CFTR-Related Disorders | CFTR | 936delTA |
| CFTR-Related Disorders | CFTR | 936delTA |
| CFTR-Related Disorders | CFTR | [delta]F311 |
| CFTR-Related Disorders | CFTR | [delta]F311 |
| CFTR-Related Disorders | CFTR | 1078delT, 1078delT |
| CFTR-Related Disorders | CFTR | 1078delT, 1078delT |
| CFTR-Related Disorders | CFTR | 1161delC, 1161delC |
| CFTR-Related Disorders | CFTR | 1161delC, 1161delC |
| CFTR-Related Disorders | CFTR | 1609delCA, 1609delCA |
| CFTR-Related Disorders | CFTR | 1609delCA, 1609delCA |
| CFTR-Related Disorders | CFTR | [delta]I507 |
| CFTR-Related Disorders | CFTR | [delta]I507 |
| CFTR-Related Disorders | CFTR | rs332, [delta]F508 |
| CFTR-Related Disorders | CFTR | rs332, [delta]F508 |
| CFTR-Related Disorders | CFTR | 1677delTA, 1677delTA |
| CFTR-Related Disorders | CFTR | 1677delTA, 1677delTA |
| CFTR-Related Disorders | CFTR | 1717−1G->A |
| CFTR-Related Disorders | CFTR | 1812−1G->A |
| CFTR-Related Disorders | CFTR | 1898+1G->A |
| CFTR-Related Disorders | CFTR | 1898+1G->T |
| CFTR-Related Disorders | CFTR | 1898+5G->T |
| CFTR-Related Disorders | CFTR | 1949del84, 1949del84 |
| CFTR-Related Disorders | CFTR | 1949del84, 1949del84 |
| CFTR-Related Disorders | CFTR | 2043delG, 2043delG |
| CFTR-Related Disorders | CFTR | 2043delG, 2043delG |
| CFTR-Related Disorders | CFTR | 2055del9->A |
| CFTR-Related Disorders | CFTR | 2055del9->A |
| CFTR-Related Disorders | CFTR | 2143delT, 2143delT |
| CFTR-Related Disorders | CFTR | 2143delT, 2143delT |
| CFTR-Related Disorders | CFTR | 2184delA, 2184delA |
| CFTR-Related Disorders | CFTR | 2184delA, 2184delA |
| CFTR-Related Disorders | CFTR | 2184insA, 2184insA |
| CFTR-Related Disorders | CFTR | 2184insA, 2184insA |
| CFTR-Related Disorders | CFTR | 2307insA, 2307insA |
| CFTR-Related Disorders | CFTR | 2307insA, 2307insA |
| CFTR-Related Disorders | CFTR | 296+12T->C |
| CFTR-Related Disorders | CFTR | 2789+5G->A |
| CFTR-Related Disorders | CFTR | 2869insG, 2869insG |
| CFTR-Related Disorders | CFTR | 2869insG, 2869insG |
| CFTR-Related Disorders | CFTR | 3120G->A |
| CFTR-Related Disorders | CFTR | 3120+1G->A |
| CFTR-Related Disorders | CFTR | 3272−26A->G |
| CFTR-Related Disorders | CFTR | 3659delC, 3659delC |
| CFTR-Related Disorders | CFTR | 3659delC, 3659delC |
| CFTR-Related Disorders | CFTR | 3667del4, 3667del4 |
| CFTR-Related Disorders | CFTR | 3667del4, 3667del4 |
| CFTR-Related Disorders | CFTR | 3791delC, 3791delC |
| CFTR-Related Disorders | CFTR | 3791delC, 3791delC |
| CFTR-Related Disorders | CFTR | 3821delT, 3821delT |
| CFTR-Related Disorders | CFTR | 3821delT, 3821delT |
| CFTR-Related Disorders | CFTR | 3905insT, 3905insT |
| CFTR-Related Disorders | CFTR | 3905insT, 3905insT |
| CFTR-Related Disorders | CFTR | 4016insT, 4016insT |
| CFTR-Related Disorders | CFTR | 4016insT, 4016insT |
| CFTR-Related Disorders | CFTR | 394delTT, 394delTT |
| CFTR-Related Disorders | CFTR | 394delTT, 394delTT |
| CFTR-Related Disorders | CFTR | 405+1G->A |
| CFTR-Related Disorders | CFTR | 405+3A->C |
| CFTR-Related Disorders | CFTR | 444delA |
| CFTR-Related Disorders | CFTR | 444delA |
| CFTR-Related Disorders | CFTR | 3876delA, 3876delA |
| CFTR-Related Disorders | CFTR | 3876delA, 3876delA |
| CFTR-Related Disorders | CFTR | 457TAT->G |
| CFTR-Related Disorders | CFTR | 457TAT->G |
| CFTR-Related Disorders | CFTR | 3199del6, 3199del6 |
| CFTR-Related Disorders | CFTR | 3199del6, 3199del6 |
| CFTR-Related Disorders | CFTR | 406-1G->A |
| CFTR-Related Disorders | CFTR | 663delT, 663delT |
| CFTR-Related Disorders | CFTR | 663delT, 663delT |
| CFTR-Related Disorders | CFTR | 935delA, 935delA |
| CFTR-Related Disorders | CFTR | 935delA, 935delA |
| CFTR-Related Disorders | CFTR | CFTR dele2,3 (21 kb) |
| CFTR-Related Disorders | CFTR | CFTR dele2,3 (21 kb) |
| CFTR-Related Disorders | CFTR | D1152H |
| CFTR-Related Disorders | CFTR | E60X |
| CFTR-Related Disorders | CFTR | E92X |
| CFTR-Related Disorders | CFTR | F508C, rs1800093 |
| CFTR-Related Disorders | CFTR | G178R |
| CFTR-Related Disorders | CFTR | G330X |
| CFTR-Related Disorders | CFTR | G480C |
| CFTR-Related Disorders | CFTR | G542X |
| CFTR-Related Disorders | CFTR | G551D |
| CFTR-Related Disorders | CFTR | G622D |
| CFTR-Related Disorders | CFTR | G85E |
| CFTR-Related Disorders | CFTR | G91R |
| CFTR-Related Disorders | CFTR | I148T, rs35516286 |
| CFTR-Related Disorders | CFTR | I506V |
| CFTR-Related Disorders | CFTR | IVS8-5T |
| CFTR-Related Disorders | CFTR | IVS8-7T |
| CFTR-Related Disorders | CFTR | IVS8-9T |
| CFTR-Related Disorders | CFTR | K710X |
| CFTR-Related Disorders | CFTR | L206W |
| CFTR-Related Disorders | CFTR | M1101K, rs36210737 |
| CFTR-Related Disorders | CFTR | N1303K |
| CFTR-Related Disorders | CFTR | P574H |
| CFTR-Related Disorders | CFTR | Q1238X |
| CFTR-Related Disorders | CFTR | Q359K/T360K_wt |
| CFTR-Related Disorders | CFTR | Q493X |
| CFTR-Related Disorders | CFTR | Q552X |
| CFTR-Related Disorders | CFTR | Q890X |
| CFTR-Related Disorders | CFTR | R1066C |
| CFTR-Related Disorders | CFTR | R1070Q |
| CFTR-Related Disorders | CFTR | R1158X |
| CFTR-Related Disorders | CFTR | R1162X |
| CFTR-Related Disorders | CFTR | R117C |
| CFTR-Related Disorders | CFTR | R117H |
| CFTR-Related Disorders | CFTR | R1283M |
| CFTR-Related Disorders | CFTR | R334W |
| CFTR-Related Disorders | CFTR | R347H |
| CFTR-Related Disorders | CFTR | R347P |
| CFTR-Related Disorders | CFTR | R352Q |
| CFTR-Related Disorders | CFTR | R553X |
| CFTR-Related Disorders | CFTR | R560T |
| CFTR-Related Disorders | CFTR | R709X |
| CFTR-Related Disorders | CFTR | R75X |
| CFTR-Related Disorders | CFTR | R764X |
| CFTR-Related Disorders | CFTR | S1196X |
| CFTR-Related Disorders | CFTR | S1235R, rs34911792 |
| CFTR-Related Disorders | CFTR | S1251N |
| CFTR-Related Disorders | CFTR | S1255X |
| CFTR-Related Disorders | CFTR | S364P |
| CFTR-Related Disorders | CFTR | S549I |
| CFTR-Related Disorders | CFTR | S549N |
| CFTR-Related Disorders | CFTR | S549R |
| CFTR-Related Disorders | CFTR | S549R |
| CFTR-Related Disorders | CFTR | T338I |
| CFTR-Related Disorders | CFTR | V520F |
| CFTR-Related Disorders | CFTR | W1089X |
| CFTR-Related Disorders | CFTR | W1204X |
| CFTR-Related Disorders | CFTR | W1204X |
| CFTR-Related Disorders | CFTR | W1282X |
| CFTR-Related Disorders | CFTR | Y1092X |
| CFTR-Related Disorders | CFTR | Y122X |
| Choroideremia | CHM | c.1609+2dupT |
| Choroideremia | CHM | c.1609+2dupT |
| CLN3-Related Neuronal Ceroid- | CLN3 | c.461_677del |
| Lipofuscinosis | | |
| CLN3-Related Neuronal Ceroid- | CLN3 | c.461_677del |
| Lipofuscinosis | | |
| CLN3-Related Neuronal Ceroid- | CLN3 | c.791_1056del |
| Lipofuscinosis | | |
| CLN3-Related Neuronal Ceroid- | CLN3 | c.791_1056del |
| Lipofuscinosis | | |
| CLN5-Related Neuronal Ceroid- | CLN5 | c.1175_1176delAT |
| Lipofuscinosis | | |
| CLN5-Related Neuronal Ceroid- | CLN5 | c.1175_1176delAT |
| Lipofuscinosis | | |
| CLN5-Related Neuronal Ceroid- | CLN5 | c.225G > A |
| Lipofuscinosis | | |
| CLN8-Related Neuronal Ceroid- | CLN8 | c.70C > G |
| Lipofuscinosis | | |
| Cohen Syndrome | VPS13B | c.3348_3349delCT |
| Cohen Syndrome | VPS13B | c.3348_3349delCT |
| Congenital Cataracts, Facial | CTDP1 | IVS6+389C > T |
| Dysmorphism, and Neuropathy | | |
| Congenital Disorder of Glycosylation | PMM2 | p.F119L |
| Ia | | |
| Congenital Disorder of Glycosylation | PMM2 | p.R141H |
| Ia | | |
| Congenital Disorder of Glycosylation | MPI | R295H, rs28928906 |
| Ib | | |
| Congenital Finnish Nephrosis | NPHS1 | c.121_122del |
| Congenital Finnish Nephrosis | NPHS1 | c.121_122del |
| Congenital Finnish Nephrosis | NPHS1 | c.3325C > T |
| Crohn Disease | NOD2 | 3020 ins C |
| Crohn Disease | NOD2 | 3020 ins C |
| Crohn Disease | NOD2 | G908R, rs2066845 |
| Crohn Disease | NOD2 | R702W, rs2066844 |
| Cystinosis | CTNS | 1035insC |
| Cystinosis | CTNS | 1035insC |
| Cystinosis | CTNS | 537del21 |
| Cystinosis | CTNS | 537del21 |
| Cystinosis | CTNS | 57 kb deletion |
| Cystinosis | CTNS | 57 kb deletion |
| Cystinosis | CTNS | D205N |
| Cystinosis | CTNS | L158P |
| Cystinosis | CTNS | W138X |
| DFNA 9 (COCH) | COCH | P51S |
| Diabetes and Hearing Loss | mtDNA | 3234A > G |
| Diabetes and Hearing Loss | mtDNA | 3271T > C |
| Diabetes and Hearing Loss | mtDNA | G8363A |
| Diabetes and Hearing Loss | mtDNA | T14709C |
| Early-Onset Primary Dystonia (DYT1) | TORIA | 904_906delGAG |
| Early-Onset Primary Dystonia (DYT1) | TORIA | 904_906delGAG |
| Epidermolysis Bullosa Junctional, | LAMB3 | 3024delT |
| Herlitz-Pearson Type | | |
| Epidermolysis Bullosa Junctional, | LAMB3 | 3024delT |
| Herlitz-Pearson Type | | |
| Epidermolysis Bullosa Junctional, | LAMB3 | p.Q243X |
| Herlitz-Pearson Type | | |
| Epidermolysis Bullosa Junctional, | LAMB3 | R144X |
| Herlitz-Pearson Type | | |
| Epidermolysis Bullosa Junctional, | LAMB3 | R42X |
| Herlitz-Pearson Type | | |
| Epidermolysis Bullosa Junctional, | LAMB3 | R635X |
| Herlitz-Pearson Type | | |
| Epidermolysis Bullosa Junctional, | LAMA3 | R650X |
| Herlitz-Pearson Type | | |
| Epidermolysis Bullosa Junctional, | LAMC2 | R95X |
| Herlitz-Pearson Type | | |
| Factor V Leiden Thrombophilia | F5 | H1299R |
| Factor V Leiden Thrombophilia | F5 | R506Q, rs6025 |
| Factor V R2 Mutation Thrombophilia | F5 | rs6027 |
| Factor XI Deficiency | F11 | E117X (576G > T) |
| Factor XI Deficiency | F11 | F283L (1074T > C) |
| Factor XI Deficiency | F11 | IVS14 +1G > A |
| Factor XI Deficiency | F11 | IVS14del14 |
| Factor XI Deficiency | F11 | IVS14del14 |
| Factor XIII Deficiency | F13A1 | V34L, rs5985 |
| Familial Adenomatous Polyposis | APC | I1307K, rs1801155 |
| Familial Dysautonomia | IKBKAP | 2507+6T > C |
| Familial Dysautonomia | IKBKAP | P914L |
| Familial Dysautonomia | IKBKAP | R696P |
| Familial Hypercholesterolemia Type B | APOB | R3500Q, rs5742904 |
| Familial Hypercholesterolemia Type B | APOB | R3500W |
| Familial Hypercholesterolemia Type B | APOB | R3531C, rs12713559 |
| Familial Mediterranean Fever | MEFV | A744S (2230G > T) |
| Familial Mediterranean Fever | MEFV | delI692 (del2076_2078) |
| Familial Mediterranean Fever | MEFV | delI692 (del2076_2078) |
| Familial Mediterranean Fever | MEFV | E148Q (442 G > C), rs3743930 |
| Familial Mediterranean Fever | MEFV | E167D (501 G > C) |
| Familial Mediterranean Fever | MEFV | F479L (1437 C > G) |
| Familial Mediterranean Fever | MEFV | K695R (2084A > G) |
| Familial Mediterranean Fever | MEFV | M680I (2040G > C) |
| Familial Mediterranean Fever | MEFV | M694I (2082G > A), |
| | | rs28940578 |
| Familial Mediterranean Fever | MEFV | M694V (2080A > G) |
| Familial Mediterranean Fever | MEFV | P369S (1105 C > T), |
| | | rs11466023 |
| Familial Mediterranean Fever | MEFV | R408Q (1223G > A), |
| | | rs11466024 |
| Familial Mediterranean Fever | MEFV | R653H (1958G > A) |
| Familial Mediterranean Fever | MEFV | R761H (2282G > A) |
| Familial Mediterranean Fever | MEFV | T2671 (800 C > T) |
| Familial Mediterranean Fever | MEFV | V726A (2177T > C), |
| | | rs28940579 |
| FANCC-Related Fanconi Anemia | FANCC | 322delG |
| FANCC-Related Fanconi Anemia | FANCC | 322delG |
| FANCC-Related Fanconi Anemia | FANCC | IVS4+4A > T (711 +4A > T) |
| FANCC-Related Fanconi Anemia | FANCC | Q13X (37C > T) |
| FANCC-Related Fanconi Anemia | FANCC | R547X |
| FGFR1-Related Craniosynostosis | FGFR1 | P252R |
| FGFR2-Related Craniosynostosis | FGFR2 | P253R |
| FGFR2-Related Craniosynostosis | FGFR2 | S252W |
| FGFR3-Related Craniosynostosis | FGFR3 | A391E, rs28931615 |
| FGFR3-Related Craniosynostosis | FGFR3 | P250R, rs4647924 |
| Free Sialic Acid Storage Disorders | SLC17A5 | c.1007_1008delTA |
| Free Sialic Acid Storage Disorders | SLC17A5 | c.1007_1008delTA |
| Free Sialic Acid Storage Disorders | SLC17A5 | c.115C > T |
| Frontotemporal Dementia with | MAPT | IVS10+16 |
| Parkinsonism-17 | | |
| Frontotemporal Dementia with | MAPT | P301L |
| Parkinsonism-17 | | |
| Frontotemporal Dementia with | MAPT | P301S |
| Parkinsonism-17 | | |
| Frontotemporal Dementia with | MAPT | R406W |
| Parkinsonism-17 | | |
| Fumarase deficiency | FH | c.1431_1433dupAAA |
| Fumarase deficiency | FH | c.1431_1433dupAAA |
| Galactosemia | GALT | 5.0 Kb gene deletion |
| Galactosemia | GALT | 5.0 Kb gene deletion |
| Galactosemia | GALT | 5′UTR-119del |
| Galactosemia | GALT | 5′UTR-119del |
| Galactosemia | GALT | IVS2-2 A > G |
| Galactosemia | GALT | K285N |
| Galactosemia | GALT | L195P T > C |
| Galactosemia | GALT | L218L |
| Galactosemia | GALT | N314D, rs2070074 |
| Galactosemia | GALT | Phel 71Ser |
| Galactosemia | GALT | Q169K |
| Galactosemia | GALT | Q188R |
| Galactosemia | GALT | S135L |
| Galactosemia | GALT | T138M C > T |
| Galactosemia | GALT | X380R |
| Galactosemia | GALT | Y209C A > G |
| Gaucher Disease | GBA | 1035insG |
| Gaucher Disease | GBA | 1035insG |
| Gaucher Disease | GBA | 84insG |
| Gaucher Disease | GBA | 84insG |
| Gaucher Disease | GBA | D409H, rs1064651 |
| Gaucher Disease | GBA | D409V |
| Gaucher Disease | GBA | IVS2(+1)G > A |
| Gaucher Disease | GBA | L444P (1448T > C), rs35095275 |
| Gaucher Disease | GBA | N370S |
| Gaucher Disease | GBA | R463C |
| Gaucher Disease | GBA | R463H |
| Gaucher Disease | GBA | R496H (1604G > A) |
| Gaucher Disease | GBA | V394L |
| GJB2-Related DFNA 3 Nonsyndromic | GJB2 | 167delT |
| Hearing Loss and Deafness | | |
| GJB2-Related DFNA 3 Nonsyndromic | GJB2 | 167delT |
| Hearing Loss and Deafness | | |
| GJB2-Related DFNA 3 Nonsyndromic | GJB2 | 235delC |
| Hearing Loss and Deafness | | |
| GJB2-Related DFNA 3 Nonsyndromic | GJB2 | 235delC |
| Hearing Loss and Deafness | | |
| GJB2-Related DFNA 3 Nonsyndromic | GJB2 | 35delG |
| Hearing Loss and Deafness | | |
| GJB2-Related DFNA 3 Nonsyndromic | GJB2 | 35delG |
| Hearing Loss and Deafness | | |
| GJB2-Related DFNA 3 Nonsyndromic | GJB2 | IVS1+1G > A |
| Hearing Loss and Deafness | | |
| GJB2-Related DFNB 1 Nonsyndromic | GJB2 | 101delAG |
| Hearing Loss and Deafness | | |
| GJB2-Related DFNB 1 Nonsyndromic | GJB2 | 313del14 |
| Hearing Loss and Deafness | | |
| GJB2-Related DFNB 1 Nonsyndromic | GJB2 | 313del14 |
| Hearing Loss and Deafness | | |
| GJB2-Related DFNB 1 Nonsyndromic | GJB2 | delE120 |
| Hearing Loss and Deafness | | |
| GJB2-Related DFNB 1 Nonsyndromic | GJB2 | delE120 |
| Hearing Loss and Deafness | | |
| GJB2-Related DFNB 1 Nonsyndromic | GJB2 | M34T, rs35887622 |
| Hearing Loss and Deafness | | |
| GJB2-Related DFNB 1 Nonsyndromic | GJB2 | Q124X |
| Hearing Loss and Deafness | | |
| GJB2-Related DFNB 1 Nonsyndromic | GJB2 | R184P |
| Hearing Loss and Deafness | | |
| GJB2-Related DFNB 1 Nonsyndromic | GJB2 | V37I |
| Hearing Loss and Deafness | | |
| GJB2-Related DFNB 1 Nonsyndromic | GJB2 | W24X |
| Hearing Loss and Deafness | | |
| GJB2-Related DFNB 1 Nonsyndromic | GJB2 | W77R |
| Hearing Loss and Deafness | | |
| GJB2-Related DFNB 1 Nonsyndromic | GJB2 | W77X |
| Hearing Loss and Deafness | | |
| Glucose-6-Phosphate Dehydrogenase | G6PD | A335V |
| Deficiency | | |
| Glucose-6-Phosphate Dehydrogenase | G6PD | R459L |
| Deficiency | | |
| Glucose-6-Phosphate Dehydrogenase | G6PD | R459P |
| Deficiency | | |
| Glucose-6-Phosphate Dehydrogenase | G6PD | rs1050828, rs1050828 |
| Deficiency | | |
| Glucose-6-Phosphate Dehydrogenase | G6PD | rs1050829, rs1050829 |
| Deficiency | | |
| Glucose-6-Phosphate Dehydrogenase | G6PD | rs5030868, rs5030868 |
| Deficiency | | |
| Glutaricacidemia Type 1 | GCDH | A421V |
| Glutaricacidemia Type 1 | GCDH | R402W |
| Glycogen Storage Disease Type la | G6PC | 459insTA |
| Glycogen Storage Disease Type la | G6PC | 459insTA |
| Glycogen Storage Disease Type la | G6PC | 727G/T |
| Glycogen Storage Disease Type la | G6PC | del F327 |
| Glycogen Storage Disease Type la | G6PC | del F327 |
| Glycogen Storage Disease Type la | G6PC | G188R |
| Glycogen Storage Disease Type la | G6PC | G270V |
| Glycogen Storage Disease Type la | G6PC | Q242X |
| Glycogen Storage Disease Type la | G6PC | Q27fsdelC |
| Glycogen Storage Disease Type la | G6PC | Q27fsdelC |
| Glycogen Storage Disease Type la | G6PC | Q347X |
| Glycogen Storage Disease Type la | G6PC | R83C |
| Glycogen Storage Disease Type la | G6PC | R83H |
| Glycogen Storage Disease Type 1b | G6PT1 | 1211delCT |
| Glycogen Storage Disease Type 1b | G6PT1 | A367T |
| Glycogen Storage Disease Type 1b | G6PT1 | G339C |
| Glycogen Storage Disease Type 1b | G6PT1 | G339D |
| Glycogen Storage Disease Type 1b | G6PT1 | W118R |
| Glycogen Storage Disease Type II | GAA | Arg854X |
| Glycogen Storage Disease Type II | GAA | Asp645Glu, rs28940868 |
| Glycogen Storage Disease Type II | GAA | IVS1(−13t > g) |
| Glycogen Storage Disease Type III | AGL | 1484delT |
| Glycogen Storage Disease Type III | AGL | 1484delT |
| Glycogen Storage Disease Type III | AGL | 17delAG |
| Glycogen Storage Disease Type III | AGL | 17delAG |
| Glycogen Storage Disease Type III | AGL | Q6X |
| Glycogen Storage Disease Type V | PYGM | G204S |
| Glycogen Storage Disease Type V | PYGM | K542T |
| Glycogen Storage Disease Type V | PYGM | K542X |
| Glycogen Storage Disease Type V | PYGM | R49X |
| GNE-Related Myopathies | GNE | M712T, rs28937594 |
| Gracile Syndrome | BCS1L | c.232A > G, rs28937590 |
| Hemoglobin S Beta-Thalassemia | HBB | c.19G > A |
| Hemoglobin S Beta-Thalassemia | HBB | c.20A > T |
| Hemoglobin S Beta-Thalassemia | HBB | c.79G > A |
| Hemoglobin S Beta-Thalassemia | HBB | Hb CS |
| Hemoglobin S Beta-Thalassemia | HBB | Hb D |
| Hemoglobin S Beta-Thalassemia | HBB | Hb O |
| Hereditary Fructose Intolerance | ALDOB | A149P, rs1800546 |
| Hereditary Fructose Intolerance | ALDOB | A174D |
| Hereditary Fructose Intolerance | ALDOB | Delta4E4 |
| Hereditary Fructose Intolerance | ALDOB | Delta4E4 |
| Hereditary Fructose Intolerance | ALDOB | N334K |
| Hereditary Fructose Intolerance | ALDOB | Y203X |
| Hereditary Pancreatitis | PRSS1 | A16V |
| Hereditary Pancreatitis | SPINK1 | MIT |
| Hereditary Pancreatitis | PRSS1 | N29I |
| Hereditary Pancreatitis | SPINKI | N34S, rs17107315 |
| Hereditary Pancreatitis | PRSS1 | R122C |
| Hereditary Pancreatitis | PRSS1 | R122H |
| Hereditary Thymine-Uraciluria | DPYD | rs3918290 |
| Hexosaminidase A Deficiency | HEXA | 1278insTATC |
| Hexosaminidase A Deficiency | HEXA | 1278insTATC |
| Hexosaminidase A Deficiency | HEXA | G269S (805G > A) |
| Hexosaminidase A Deficiency | HEXA | IVS12 +1G > C |
| Hexosaminidase A Deficiency | HEXA | IVS7 +1G > A |
| Hexosaminidase A Deficiency | HEXA | IVS9 +1G > A |
| Hexosaminidase A Deficiency | HEXA | R178C |
| Hexosaminidase A Deficiency | HEXA | R178H |
| Hexosaminidase A Deficiency | HEXA | R247W (739C > T) |
| Hexosaminidase A Deficiency | HEXA | R249W (745C > T) |
| HFE-Associated Hereditary | HFE | E168Q |
| Hemochromatosis | | |
| HFE-Associated Hereditary | HFE | E168X |
| Hemochromatosis | | |
| HFE-Associated Hereditary | HFE | HM971246, H63H |
| Hemochromatosis | | |
| HFE-Associated Hereditary | HFE | P160delC |
| Hemochromatosis | | |
| HFE-Associated Hereditary | HFE | P160delC |
| Hemochromatosis | | |
| HFE-Associated Hereditary | HFE | Q127H, rs28934595 |
| Hemochromatosis | | |
| HFE-Associated Hereditary | HFE | Q283P |
| Hemochromatosis | | |
| HFE-Associated Hereditary | HFE | rs1799945, rs1799945 |
| Hemochromatosis | | |
| HFE-Associated Hereditary | HFE | rs1800562, rs1800562 |
| Hemochromatosis | | |
| HFE-Associated Hereditary | HFE | rs1800730, rs1800730 |
| Hemochromatosis | | |
| HFE-Associated Hereditary | HFE | V53M |
| Hemochromatosis | | |
| HFE-Associated Hereditary | HFE | V59M |
| Hemochromatosis | | |
| HFE-Associated Hereditary | HFE | W169X |
| Hemochromatosis | | |
| Hidrotic Ectodermal Dysplasia 2 | GJB6 | A88V, rs28937872 |
| Hidrotic Ectodermal Dysplasia 2 | GJB6 | G11R |
| Hidrotic Ectodermal Dysplasia 2 | GJB6 | V37E |
| Homocystinuria Caused by | CBS | G307S 919G->A |
| Cystathionine Beta-Synthase | | |
| Deficiency | | |
| Homocystinuria Caused by | CBS | I278T 833T->C, rs5742905 |
| Cystathionine Beta-Synthase | | |
| Deficiency | | |
| Hyperkalemic Periodic Paralysis Type | SCN4A | I693T |
| 1 | | |
| Hyperkalemic Periodic Paralysis Type | SCN4A | L689I |
| 1 | | |
| Hyperkalemic Periodic Paralysis Type | SCN4A | L689V |
| 1 | | |
| Hyperkalemic Periodic Paralysis Type | SCN4A | M1360V |
| 1 | | |
| Hyperkalemic Periodic Paralysis Type | SCN4A | M1592V |
| 1 | | |
| Hyperkalemic Periodic Paralysis Type | SCN4A | p.A1156T |
| 1 | | |
| Hyperkalemic Periodic Paralysis Type | SCN4A | p.M1370V |
| 1 | | |
| Hyperkalemic Periodic Paralysis Type | SCN4A | p.R1448C |
| 1 | | |
| Hyperkalemic Periodic Paralysis Type | SCN4A | p.T1313M |
| 1 | | |
| Hyperkalemic Periodic Paralysis Type | SCN4A | R675G |
| 1 | | |
| Hyperkalemic Periodic Paralysis Type | SCN4A | R675Q |
| 1 | | |
| Hyperkalemic Periodic Paralysis Type | SCN4A | R675W |
| 1 | | |
| Hyperkalemic Periodic Paralysis Type | SCN4A | T704M |
| 1 | | |
| Hyperkalemic Periodic Paralysis Type | SCN4A | V781I |
| 1 | | |
| Hyperornithinemia-Hyperammonemia- | SLC25A15 | F188del |
| Homocitrullinuria Syndrome | | |
| Hyperornithinemia-Hyperammonemia- | SLC25A15 | F188del |
| Homocitrullinuria Syndrome | | |
| Hyperoxaluria, Primary, Type 1 | AGXT | 33insC |
| Hyperoxaluria, Primary, Type 1 | AGXT | 33insC |
| Hyperoxaluria, Primary, Type 1 | AGXT | F152I |
| Hyperoxaluria, Primary, Type 1 | AGXT | G170R |
| Hyperoxaluria, Primary, Type 1 | AGXT | I244T |
| Hyperoxaluria, Primary, Type 2 | GRHPR | 103delG |
| Hyperoxaluria, Primary, Type 2 | GRHPR | 103delG |
| Hypochondroplasia | FGFR3 | Asn328Ile |
| Hypochondroplasia | FGFR3 | I538V |
| Hypochondroplasia | FGFR3 | K650M |
| Hypochondroplasia | FGFR3 | K650N 1950G > T |
| Hypochondroplasia | FGFR3 | K650Q |
| Hypochondroplasia | FGFR3 | N540K 1620C > A |
| Hypochondroplasia | FGFR3 | N540S |
| Hypochondroplasia | FGFR3 | N540T |
| Hypokalemic Periodic Paralysis Type 1 | CACNAIS | R528G |
| Hypokalemic Periodic Paralysis Type 1 | CACNAIS | R528H |
| Hypokalemic Periodic Paralysis Type 1 | CACNAIS | rs28930068, rs28930068 |
| Hypokalemic Periodic Paralysis Type 1 | CACNAIS | rs28930069, rs28930069 |
| Hypokalemic Periodic Paralysis Type 2 | SCN4A | R669H |
| Hypokalemic Periodic Paralysis Type 2 | SCN4A | R672C |
| Hypokalemic Periodic Paralysis Type 2 | SCN4A | R672G |
| Hypokalemic Periodic Paralysis Type 2 | SCN4A | R672H |
| Hypokalemic Periodic Paralysis Type 2 | SCN4A | R672S |
| Hypophosphatasia | ALPL | Asp361Val |
| Hypophosphatasia | ALPL | c.1559delT |
| Hypophosphatasia | ALPL | c.1559delT |
| Hypophosphatasia | ALPL | E174K |
| Hypophosphatasia | ALPL | G317D |
| Hypophosphatasia | ALPL | Phe310Leu |
| Isovaleric Acidemia | IVD | A282V |
| Isovaleric Acidemia | IVD | rs28940889 |
| Krabbe Disease | GALC | EX11-17DEL |
| Krabbe Disease | GALC | EX11-17DEL |
| Krabbe Disease | GALC | G270D |
| Krabbe Disease | GALC | rs1805078, rs1805078 |
| Krabbe Disease | GALC | rs398607 |
| Leber Hereditary Optic Neuropathy | mtDNA | 14484T > C |
| Leber Hereditary Optic Neuropathy | mtDNA | 15257G > A |
| Leber Hereditary Optic Neuropathy | mtDNA | G14459A |
| Leber Hereditary Optic Neuropathy | mtDNA | G3460A |
| Leber Hereditary Optic Neuropathy | mtDNA | m.11778G > A |
| Leber Hereditary Optic Neuropathy | mtDNA | m.13708G > A |
| Leber Hereditary Optic Neuropathy | mtDNA | m.15812G > A |
| Leber Hereditary Optic Neuropathy | mtDNA | m.3394T > C |
| Leber Hereditary Optic Neuropathy | mtDNA | m.4216T > C |
| Leber Hereditary Optic Neuropathy | mtDNA | m.4917A > G |
| Leigh Syndrome, French-Canadian | LRPPRC | A354V |
| Type | | |
| LGMD2I | FKRP | L276I, rs28937900 |
| Long Chain 3-Hydroxyacyl-CoA | HADHA | E474Q c.1528G > C |
| Dehydrogenase Deficiency | | |
| Long Chain 3-Hydroxyacyl-CoA | HADHA | Q342X 1132C > T |
| Dehydrogenase Deficiency | | |
| Maple Syrup Urine Disease Type 1A | BCKDHA | Y438N |
| Maple Syrup Urine Disease Type 1B | BCKDHB | E372X |
| Maple Syrup Urine Disease Type 1B | BCKDHB | G278S |
| Maple Syrup Urine Disease Type 1B | BCKDHB | R183P |
| McCune-Albright Syndrome | GNAS | R201C |
| McCune-Albright Syndrome | GNAS | R201G |
| McCune-Albright Syndrome | GNAS | R201H |
| Medium Chain Acyl-Coenzyme A | ACADM | 244insT |
| Dehydrogenase Deficiency | | |
| Medium Chain Acyl-Coenzyme A | ACADM | 244insT |
| Dehydrogenase Deficiency | | |
| Medium Chain Acyl-Coenzyme A | ACADM | 250C > T |
| Dehydrogenase Deficiency | | |
| Medium Chain Acyl-Coenzyme A | ACADM | 583G > A |
| Dehydrogenase Deficiency | | |
| Medium Chain Acyl-Coenzyme A | ACADM | 616C > T |
| Dehydrogenase Deficiency | | |
| Medium Chain Acyl-Coenzyme A | ACADM | 617G > A |
| Dehydrogenase Deficiency | | |
| Medium Chain Acyl-Coenzyme A | ACADM | 799G > A |
| Dehydrogenase Deficiency | | |
| Medium Chain Acyl-Coenzyme A | ACADM | K304E |
| Dehydrogenase Deficiency | | |
| Medium Chain Acyl-Coenzyme A | ACADM | Y42H |
| Dehydrogenase Deficiency | | |
| Megalencephalic | MLC1 | 135insC |
| Leukoencephalopathy with Subcortical | | |
| Cysts | | |
| Megalencephalic | MLC1 | 135insC |
| Leukoencephalopathy with Subcortical | | |
| Cysts | | |
| MELAS | mtDNA | 3243A > G |
| MELAS | mtDNA | 3250T > C |
| MELAS | mtDNA | 3252A > G |
| MELAS | mtDNA | A12770G |
| MELAS | mtDNA | C3256T |
| MELAS | mtDNA | G13513A |
| MELAS | mtDNA | T3291C |
| MELAS | mtDNA | T8356C |
| MELAS | mtDNA | T9957C |
| MERRF | mtDNA | 8361G > A |
| MERRF | mtDNA | A8296G |
| MERRF | mtDNA | m.8344A > G |
| Metachromatic Leukodystrophy | ARSA | c.459+1G > A |
| Metachromatic Leukodystrophy | ARSA | p.P426L, rs28940893 |
| Metachromatic Leukodystrophy | ARSA | p.T274M |
| Metachromatic Leukodystrophy | ARSA | P377L |
| Mitochondrial Cardiomyopathy | mtDNA | A3260T |
| Mitochondrial Cardiomyopathy | mtDNA | A4300G |
| Mitochondrial Cardiomyopathy | mtDNA | C3303T |
| Mitochondrial Cardiomyopathy | mtDNA | T9997C |
| Mitochondrial DNA-Associated Leigh | mtDNA | 5537insT |
| Syndrome and NARP | | |
| Mitochondrial DNA-Associated Leigh | mtDNA | 5537insT |
| Syndrome and NARP | | |
| Mitochondrial DNA-Associated Leigh | mtDNA | 8993T > C |
| Syndrome and NARP | | |
| Mitochondrial DNA-Associated Leigh | mtDNA | 8993T > G |
| Syndrome and NARP | | |
| Mitochondrial DNA-Associated Leigh | mtDNA | C11777A |
| Syndrome and NARP | | |
| Mitochondrial DNA-Associated Leigh | mtDNA | T10158C |
| Syndrome and NARP | | |
| Mitochondrial DNA-Associated Leigh | mtDNA | T10191C |
| Syndrome and NARP | | |
| Mitochondrial DNA-Associated Leigh | mtDNA | T8851C |
| Syndrome and NARP | | |
| Mitochondrial DNA-Associated Leigh | mtDNA | T9176C |
| Syndrome and NARP | | |
| Mitochondrial DNA-Associated Leigh | mtDNA | T9176G |
| Syndrome and NARP | | |
| MTHFR Deficiency | MTHFR | 1298A > C |
| MTHFR Deficiency | MTHFR | rs1801133, rs1801133 |
| MTRNR1-Related Hearing Loss and | mtDNA | 1095T > C |
| Deafness | | |
| MTRNR1-Related Hearing Loss and | mtDNA | 1494C > T |
| Deafness | | |
| MTRNR1-Related Hearing Loss and | mtDNA | 1555A > G |
| Deafness | | |
| MTRNR1-Related Hearing Loss and | mtDNA | 961T > G |
| Deafness | | |
| MTRNR1-Related Hearing Loss and | mtDNA | A7445G |
| Deafness | | |
| MTTS1-Related Hearing Loss and | mtDNA | 7443A > G |
| Deafness | | |
| MTTS1-Related Hearing Loss and | mtDNA | 7444G > A |
| Deafness | | |
| MTTS1-Related Hearing Loss and | mtDNA | 7472insC |
| Deafness | | |
| MTTS1-Related Hearing Loss and | mtDNA | 7472insC |
| Deafness | | |
| MTTS1-Related Hearing Loss and | mtDNA | 7510T > C |
| Deafness | | |
| MTTS1-Related Hearing Loss and | mtDNA | 7511T > C |
| Deafness | | |
| MTTS1-Related Hearing Loss and | mtDNA | 7512T > C |
| Deafness | | |
| Mucolipidosis IV | MCOLN1 | delEx1 3 Ex7 (511 > 6944del) |
| Mucolipidosis IV | MCOLN1 | delEx1 3 Ex7 (511 > 6944del) |
| Mucolipidosis IV | MCOLNI | IVS-2A > G |
| Mucopolysaccharidosis Type I | IDUA | c.46_57del |
| Mucopolysaccharidosis Type I | IDUA | c.46_57del |
| Mucopolysaccharidosis Type I | IDUA | p.A327P |
| Mucopolysaccharidosis Type I | IDUA | p.P533R |
| Mucopolysaccharidosis Type I | IDUA | Q70X |
| Mucopolysaccharidosis Type I | IDUA | W402X |
| Mucopolysaccharidosis Type IIIA | SGSH | p.R245H |
| Mucopolysaccharidosis Type IIIA | SGSH | p.R74C |
| Mucopolysaccharidosis Type IIIA | SGSH | p.S66W |
| Mucopolysaccharidosis Type VII | GUSB | p.D152N |
| Multiple Endocrine Neoplasia Type 2 | RET | 2047T > A |
| Multiple Endocrine Neoplasia Type 2 | RET | 2047T > A |
| Multiple Endocrine Neoplasia Type 2 | RET | 2047T > C |
| Multiple Endocrine Neoplasia Type 2 | RET | 2047T > G |
| Multiple Endocrine Neoplasia Type 2 | RET | 2048G > A |
| Multiple Endocrine Neoplasia Type 2 | RET | A883F 2647 G > T |
| Multiple Endocrine Neoplasia Type 2 | RET | Glu768Asp G > C |
| Multiple Endocrine Neoplasia Type 2 | RET | M918T |
| Muscle-Eye-Brain Disease | POMGNT1 | c.1539+1G > A |
| MYH-Associated Polyposis | MUTYH | c.1376C > A |
| MYH-Associated Polyposis | MUTYH | c.494A > G, rs34612342 |
| MYH-Associated Polyposis | GENE SYMBOL | rs36053993, rs36053993 |
| | TBD | |
| Niemann-Pick Disease Due to | SMPD1 | c.990delC |
| Sphingomyelinase Deficiency | | |
| Niemann-Pick Disease Due to | SMPD1 | c.990delC |
| Sphingomyelinase Deficiency | | |
| Niemann-Pick Disease Due to | SMPD1 | fsP330 |
| Sphingomyelinase Deficiency | | |
| Niemann-Pick Disease Due to | SMPD1 | fsP330 |
| Sphingomyelinase Deficiency | | |
| Niemann-Pick Disease Due to | SMPD1 | L302P |
| Sphingomyelinase Deficiency | | |
| Niemann-Pick Disease Due to | SMPD1 | R496L |
| Sphingomyelinase Deficiency | | |
| Niemann-Pick Disease Due to | SMPD1 | R608del |
| Sphingomyelinase Deficiency | | |
| Niemann-Pick Disease Type C1 | NPC1 | I1061T |
| Nijmegen Breakage Syndrome | NBN | 657del5 |
| Nijmegen Breakage Syndrome | NBN | 657del5 |
| Pallister-Hall Syndrome | GLI3 | 2012delG |
| Pallister-Hall Syndrome | GLI3 | 2012delG |
| Pallister-Hall Syndrome | GLI3 | 2023delG |
| Pallister-Hall Syndrome | GLI3 | 2023delG |
| Pendred Syndrome | SLC26A4 | 1197delT |
| Pendred Syndrome | SLC26A4 | 1197delT |
| Pendred Syndrome | SLC26A4 | E384G |
| Pendred Syndrome | SLC26A4 | IV58+1(G->A) |
| Pendred Syndrome | SLC26A4 | L236P |
| Pendred Syndrome | SLC26A4 | T416P |
| Peroxisomal Bifunctional Enzyme | HSD17B4 | c.302+1G > C |
| Deficiency | | |
| Peroxisomal Bifunctional Enzyme | HSD17B4 | c.303−1G > A |
| Deficiency | | |
| Pervasive Developmental Disorders | NLGN4X | D396X |
| Pervasive Developmental Disorders | NLGN4X | D396X |
| Pervasive Developmental Disorders | NLGN4X | NLGN4X:1253delAG |
| Pervasive Developmental Disorders | NLGN4X | NLGN4X:1253delAG |
| Pervasive Developmental Disorders | NLGN3 | R451C |
| Phenylalanine Hydroxylase Deficiency | PAH | G272X |
| Phenylalanine Hydroxylase Deficiency | PAH | I65T |
| Phenylalanine Hydroxylase Deficiency | PAH | IVS12+1G > T |
| Phenylalanine Hydroxylase Deficiency | PAH | L48S, rs5030841 |
| Phenylalanine Hydroxylase Deficiency | PAH | R158Q, rs5030843 |
| Phenylalanine Hydroxylase Deficiency | PAH | R252W, rs5030847 |
| Phenylalanine Hydroxylase Deficiency | PAH | R261Q, rs5030849 |
| Phenylalanine Hydroxylase Deficiency | PAH | R408Q, rs5030859 |
| Phenylalanine Hydroxylase Deficiency | PAH | R408W, rs5030858 |
| Phenylalanine Hydroxylase Deficiency | PAH | rs5030855 |
| Phenylalanine Hydroxylase Deficiency | PAH | rs5030861 |
| Phenylalanine Hydroxylase Deficiency | PAH | Y414C, rs5030860 |
| Plasminogen Activator Inhibitor I | SERPINE1 | −844 G > A |
| Plasminogen Activator Inhibitor I | SERPINE1 | 4G/5G |
| Polycystic Kidney Disease, Autosomal | PKHD1 | c.10412T > G |
| Recessive | | |
| Polycystic Kidney Disease, Autosomal | PKHD1 | c.107C > T |
| Recessive | | |
| Polycystic Kidney Disease, Autosomal | PKHD1 | c.1486C > T |
| Recessive | | |
| Polycystic Kidney Disease, Autosomal | PKHD1 | c.5895dupA |
| Recessive | | |
| Polycystic Kidney Disease, Autosomal | PKHD1 | c.5895dupA |
| Recessive | | |
| Polycystic Kidney Disease, Autosomal | PKHD1 | c.9689del A |
| Recessive | | |
| Polycystic Kidney Disease, Autosomal | PKHD1 | c.9689del A |
| Recessive | | |
| PPT1-Related Neuronal Ceroid- | PPT1 | c.364A > T |
| Lipofuscinosis | | |
| PPT1-Related Neuronal Ceroid- | PPT1 | p.L10X |
| Lipofuscinosis | | |
| PPT1-Related Neuronal Ceroid- | PPT1 | p.R151X |
| Lipofuscinosis | | |
| PPT1-Related Neuronal Ceroid- | PPT1 | T75P |
| Lipofuscinosis | | |
| PROP1-related pituitary hormone | PROP1 | 301-302delAG |
| deficiency | | |
| PROP1-related pituitary hormone | PROP1 | 301-302delAG |
| deficiency | | |
| Prothrombin Thrombophilia | F2 | rs1799963 |
| Prothrombin Thrombophilia | F2 | rs6025, rs6025 |
| Pseudovitamin D Deficiency Rickets | CYP27B1 | 7bp duplication in exon8 |
| Pseudovitamin D Deficiency Rickets | CYP27B1 | 7bp duplication in exon8 |
| Pseudovitamin D Deficiency Rickets | CYP27B1 | 958delG |
| Pseudovitamin D Deficiency Rickets | CYP27B1 | 958delG |
| Rett Syndrome | MECP2 | 806delG |
| Rett Syndrome | MECP2 | 806delG |
| Rett Syndrome | MECP2 | A140V, rs28934908 |
| Rett Syndrome | MECP2 | P152R |
| Rett Syndrome | MECP2 | P225R |
| Rett Syndrome | MECP2 | R106W, rs28934907 |
| Rett Syndrome | MECP2 | R133C |
| Rett Syndrome | MECP2 | R168X |
| Rett Syndrome | MECP2 | R255X |
| Rett Syndrome | MECP2 | R270X |
| Rett Syndrome | MECP2 | R294X |
| Rett Syndrome | MECP2 | R306C, rs28935468 |
| Rett Syndrome | MECP2 | S134C |
| Rett Syndrome | MECP2 | T158M, rs28934906 |
| Rhizomelic Chondrodysplasia Punctata | PEX7 | p.A218V |
| Type 1 | | |
| Rhizomelic Chondrodysplasia Punctata | PEX7 | p.G217R |
| Type 1 | | |
| Rhizomelic Chondrodysplasia Punctata | PEX7 | p.L292X, rs1805137 |
| Type 1 | | |
| Short Chain Acyl-CoA Dehydrogenase | ACADS | c.511C > T, rs1800556 |
| Deficiency | | |
| Short Chain Acyl-CoA Dehydrogenase | ACADS | c.625G > A |
| Deficiency | | |
| Short Chain Acyl-CoA Dehydrogenase | ACADS | R107C |
| Deficiency | | |
| Shwachman-Diamond Syndrome | SBDS | 183_184TA > CT |
| Shwachman-Diamond Syndrome | SBDS | 183_184TA > CT |
| Shwachman-Diamond Syndrome | SBDS | 258+2T > C |
| Sjogren-Larsson Syndrome | ALDH3 A2 | c.943C > T |
| Smith-Lemli-Opitz Syndrome | DHCR7 | C380Y |
| Smith-Lemli-Opitz Syndrome | DHCR7 | IVS8-1G > C |
| Smith-Lemli-Opitz Syndrome | DHCR7 | L109P |
| Smith-Lemli-Opitz Syndrome | DHCR7 | L157P |
| Smith-Lemli-Opitz Syndrome | DHCR7 | R352Q |
| Smith-Lemli-Opitz Syndrome | DHCR7 | R352W |
| Smith-Lemli-Opitz Syndrome | DHCR7 | R404C |
| Smith-Lemli-Opitz Syndrome | DHCR7 | R446Q |
| Smith-Lemli-Opitz Syndrome | DHCR7 | T93M |
| Smith-Lemli-Opitz Syndrome | DHCR7 | V326L |
| Smith-Lemli-Opitz Syndrome | DHCR7 | W151X |
| Smith-Lemli-Opitz Syndrome | DHCR7 | W151X |
| Spastic Paraplegia 13 | HSPD1 | V72I |
| Sulfate Transporter-Related | SLC26A2 | 340delV |
| Osteochondrodysplasia | | |
| Sulfate Transporter-Related | SLC26A2 | 340delV |
| Osteochondrodysplasia | | |
| Sulfate Transporter-Related | SLC26A2 | c.837C > T |
| Osteochondrodysplasia | | |
| Sulfate Transporter-Related | SLC26A2 | C653S |
| Osteochondrodysplasia | | |
| Sulfate Transporter-Related | SLC26A2 | IVS1+2T > C |
| Osteochondrodysplasia | | |
| Sulfate Transporter-Related | SLC26A2 | R178X |
| Osteochondrodysplasia | | |
| TFR2-Related Hereditary | TFR2 | AVAQ594-597del |
| Hemochromatosis | | |
| TFR2-Related Hereditary | TFR2 | AVAQ594-597del |
| Hemochromatosis | | |
| TFR2-Related Hereditary | TFR2 | E60X |
| Hemochromatosis | | |
| TFR2-Related Hereditary | TFR2 | E60X |
| Hemochromatosis | | |
| TFR2-Related Hereditary | TFR2 | M172K |
| Hemochromatosis | | |
| TFR2-Related Hereditary | TFR2 | Y250X |
| Hemochromatosis | | |
| Thanatophoric Dysplasia | FGFR3 | G370C |
| Thanatophoric Dysplasia | FGFR3 | K650E |
| Thanatophoric Dysplasia | FGFR3 | R248C |
| Thanatophoric Dysplasia | FGFR3 | S249C |
| Thanatophoric Dysplasia | FGFR3 | S371C |
| Thanatophoric Dysplasia | FGFR3 | X807C A > T |
| Thanatophoric Dysplasia | FGFR3 | X807G |
| Thanatophoric Dysplasia | FGFR3 | X807L |
| Thanatophoric Dysplasia | FGFR3 | X807R |
| Thanatophoric Dysplasia | FGFR3 | X807S |
| Thanatophoric Dysplasia | FGFR3 | X807W |
| Thanatophoric Dysplasia | FGFR3 | Y373C |
| TPP1-Related Neuronal Ceroid- | TPP1 | c.509-1G > A |
| Lipofuscinosis | | |
| TPP1-Related Neuronal Ceroid- | TPP1 | c.509-1G > C |
| Lipofuscinosis | | |
| TPP1-Related Neuronal Ceroid- | TPP1 | G284V |
| Lipofuscinosis | | |
| TPP1-Related Neuronal Ceroid- | TPP1 | p.R208X |
| Lipofuscinosis | | |
| Transthyretin Amyloidosis | TTR | c.148G > A |
| Tyrosine Hydroxylase-Deficient DRD | TH | L205P |
| Tyrosine Hydroxylase-Deficient DRD | TH | R202H |
| Tyrosinemia Type I | FAH | E357X |
| Tyrosinemia Type I | FAH | IVS12+5 G > A |
| Tyrosinemia Type I | FAH | IVS7−6 T > G |
| Tyrosinemia Type I | FAH | IVS8−1G > C |
| Tyrosinemia Type I | FAH | p.W262X |
| Tyrosinemia Type I | FAH | P261L |
| Tyrosinemia Type I | FAH | Q64H |
| Wilson Disease | ATP7B | 1340del4 |
| Wilson Disease | ATP7B | 3402delC |
| Wilson Disease | ATP7B | 3402delC |
| Wilson Disease | ATP7B | H1069Q |
| Wilson Disease | ATP7B | R778G |
| Wilson Disease | ATP7B | W779X |
| Wilson Disease | ATP7B | W779X |
| X-Linked Juvenile Retinoschisis | RS1 | E72K |
| X-Linked Juvenile Retinoschisis | RS1 | G109R |
| X-Linked Juvenile Retinoschisis | RS1 | G74V |
| Zellweger Syndrome Spectrum | PEX1 | c.2097_2098insT |
| Zellweger Syndrome Spectrum | PEX1 | c.2097_2098insT |
| Zellweger Syndrome Spectrum | PEX1 | c.2916delA |
| Zellweger Syndrome Spectrum | PEX1 | c.2916delA |
| Zellweger Syndrome Spectrum | PEX1 | p.G843D |

TABLE 2

| Disease/Disorder | Gene(s) |
|---|---|
| Neoplasia | PTEN: ATM; ATR; EGFR; ERBB2; ERBB3; ERBB4; |
| | Notch1; Notch2; Notch3; Notch4; AKT, AKT2; AKT3; HIF; |
| | HIF1a: HIF3a; Met; HRG; Bcl2; PPAR alpha; PPAR |
| | gamma; WT1 (Wilms Tumor); FGF Receptor Family |
| | members (5 members: 1, 2, 3, 4, 5); CDKN2a; APC; RB |
| | (retinoblastoma); MEN1; VHL; BRCA1; BRCA2; AR |
| | (Androgen Receptor); TSG101; IGF; IGF Receptor; Igf1 (4 |
| | variants); Igf2 (3 variants); Igf 1 Receptor; Igf 2 Receptor; |
| | Bax; Bcl2; caspases family (9 members: |
| | 1, 2, 3, 4, 6, 7, 8, 9, 12); Kras; Apc |
| Age-related | Aber; Ccl2; Cc2; cp (ceruloplasmin); Timp3; catbepsinD; |
| Macular | Vldlr; Ccr2 |
| Degeneration | |
| Schizophrenia | Neuregulin1 (Nrg1); Erb4 (receptor for Neuregulin); |
| | Complexin1 (Cplx1); Tphl Tryptophan hydroxylase; Tph2 |
| | Tryptophan hydroxylase 2; Neurexin 1; GSK3; GSK3a; |
| | GSK3b |
| Trinucleotide | HTT (Huntington's Dx); SBMA/SMAX1/AR (Kennedy's |
| Repeat | Dx); FXN/X25 (Friedrich's Ataxia); ATX3 (Machado- |
| Disorders | Joseph's Dx): ATXN1 and ATXN2 (spinocerebellar |
| | ataxias): DMPK (myotonic dystrophy); Atrophin-1 and Atnl |
| | (DRPLA Dx), CBP (Creb-BP - global instability); VLDLR |
| | (Alzheimer's); Atxn7; Atxn10 |
| Fragile X | FMR2; FXRI; FXR2; mGLURS |
| Syndrome | |
| Secretase Related | APH-1 (alpha and beta); Presenilin (Psen1); nicastrin |
| Disorders | (Nestn); PEN-2 |
| Prion - related | Prp |
| disorders | |
| ALS | SOD1; ALS2; STEX; FUS; TARDBP; VEGF (VEGF-a; |
| | VEGF-b; VEGF-c) |
| Drug addiction | Prkce (alcohol); Drd2; Drd4; ABAT (alcohol); GRIA2; |
| | Grm5; Grin1; Htrlb; Grin2a; Drd3; Pdyn; Grial (alcohol) |
| Autism | Mecp2; BZRAP1; MDGA2; SemaSA; Neurexin 1; Fragile X |
| | (FMR2 (AFF2); FXR1; FXR2; Mglur5) |
| Alzheimer's Disease | E1; CHIP; UCH; UBB; Tau; LRP; PICALM; Clusterin; PS1; |
| | SORL1; CR1; Vldlr; Uba1; Uba3; CHIP28 (Aqp1, |
| | Aquaporin 1); Uchl1; Uchl3; APP |
| Inflammation | IL-10; IL-1 (IL-1a; IL-1b); IL-13; IL-17 (IL-17a (CTLA8); IL- |
| | 17b; IL-17c; IL-17d; IL-17f); II-23; Cx3cr1; ptpn22; TNFa; |
| | NOD2/CARD15 for IBD; IL-6; IL-12 (IL-12a; IL-12b); |
| | CTLA4: Cx3cl1 |
| Parkinson's Disease | x-Synuclein; DJ-1; LRRK2; Parkin; PINK1 |

In some embodiments, a method further comprises the step of diagnosing a subject based on a calling step, such as diagnosing the subject with a disease associated with a detected causal genetic variant, or reporting a likelihood that the patient has or will develop such disease. Examples of diseases, associated genes, and associated sequence variants are provided herein. In some embodiments, a result is reported via a report generator, such as described herein.

In some embodiments, one or more causal genetic variants are sequence variants associated with a particular type or stage of cancer, or of cancer having a particular characteristic (e.g. metastatic potential, drug resistance, drug responsiveness). In some embodiments, the disclosure provides methods for the determination of prognosis, such as where certain mutations are known to be associated with patient outcomes. For example, ctDNA has been shown to be a better biomarker for breast cancer prognosis than the traditional cancer antigen 53 (CA-53) and enumeration of circulating tumor cells (see e.g. Dawson, et al., N Engl J Med 368:1199 (2013)). Additionally, the methods of the present disclosure can be used in therapeutic decisions, guidance, and monitoring, as well as development and clinical trials of cancer therapies. For example, treatment efficacy can be monitored by comparing patient ctDNA samples from before, during, and after treatment with particular therapies such as molecular targeted therapies (monoclonal drugs), chemotherapeutic drugs, radiation protocols, etc. or combinations of these. For example, the ctDNA can be monitored to see if certain mutations increase or decrease, new mutations appear, etc., after treatment, which can allow a physician to alter a treatment (continue, stop, or change treatment, for example) in a much shorter period of time than afforded by methods of monitoring that track patient symptoms. In some embodiments, a method further comprises the step of diagnosing a subject based on a calling step, such as diagnosing the subject with a particular stage or type of cancer associated with a detected sequence variant, or reporting a likelihood that the patient has or will develop such cancer.

For example, for therapies that are specifically targeted to patients on the basis of molecular markers (e.g. Herceptin and her2/neu status), patients are tested to find out if certain mutations are present in their tumor, and these mutations can be used to predict response or resistance to the therapy and guide the decision whether to use the therapy. Therefore, detecting and monitoring ctDNA during the course of treatment can be very useful in guiding treatment selections. Some primary (before treatment) or secondary (after treatment) cancer mutations are found to be responsible for the resistance of cancers to some therapies (Misale et al., Nature 486(7404):532 (2012)).

A variety of sequence variants that are associated with one or more kinds of cancer that may be useful in diagnosis, prognosis, or treatment decisions are known. Suitable target sequences of oncological significance that find use in the methods of the disclosure include, but are not limited to, alterations in the TP53 gene, the ALK gene, the KRAS gene, the PIK3CA gene, the BRAF gene, the EGFR gene, and the KIT gene. A target sequence the may be specifically amplified, and/or specifically analyzed for sequence variants may be all or part of a cancer-associated gene. In some embodiments, one or more sequence variants are identified in the TP53 gene. TP53 is one of the most frequently mutated genes in human cancers, for example, TP53 mutations are found in 45% of ovarian cancers, 43% of large intestinal cancers, and 42% of cancers of the upper aerodigestive track (see e.g. M. Olivier, et, al. TP53 Mutations in Human Cancers: Origins, Consequences, and Clinical Use. Cold Spring Harb Perspect Biol. 2010 January; 2(1). Characterization of the mutation status of TP53 can aid in clinical diagnosis, provide prognostic value, and influence treatment for cancer patients. For example, TP53 mutations may be used as a predictor of a poor prognosis for patients in CNS tumors derived from glial cells and a predictor of rapid disease progression in patients with chronic lymphocytic leukemia (see e.g. McLendon R E, et al. Cancer. 2005 Oct. 15; 1 04(8): 1693-9; Dicker F, et al. Leukemia. 2009 January; 23(1):117-24). Sequence variation can occur anywhere within the gene. Thus, all or part of the TP53 gene can be evaluated herein. That is, as described elsewhere herein, when target specific components (e.g. target specific primers) are used, a plurality of TP53 specific sequences can be used, for example to amplify and detect fragments spanning the gene, rather than just one or more selected subsequences (such as mutation “hot spots”) as may be used for selected targets. Alternatively, target-specific primers may be designed that hybridize upstream or downstream of one or more selected subsequences (such a nucleotide or nucleotide region associated with an increased rate of mutation among a class of subjects, also encompassed by the term “hot spot”). Standard primers spanning such a subsequence may be designed, and/or B2B primers that hybridize upstream or downstream of such a subsequence may be designed.

In some embodiments, one or more sequence variants are identified in the all or part of the ALK gene. ALK fusions have been reported in as many as 7% of lung tumors, some of which are associated with EGFR tyrosine kinase inhibitor (TKI) resistance (see e.g. Shaw et al., J Clin Oncol. Sep. 10, 2009; 27(26): 4247-4253). Up to 2013, several different point mutations spanning across the entire ALK tyrosine kinase domain have been found in patients with secondary resistance to the ALK tyrosine kinase inhibitor (TKI) (Katayama R 2012 Sci Transl Med. 2012 Feb. 8; 4(120)). Thus, mutation detection in ALK gene can be used to aid cancer therapy decisions.

In some embodiments, one or more sequence variants are identified in the all or part of the KRAS gene. Approximately 15-25% of patients with lung adenocarcinoma and 40% of patients with colorectal cancer have been reported as harboring tumor associated KRAS mutations (see e.g. Neuman 2009, Pathol Res Pract. 2009; 205(12):858-62). Most of the mutations are located at codons 12, 13, and 61 of the KRAS gene. These mutations activate KRAS signaling pathways, which trigger growth and proliferation of tumor cells. Some studies indicate that patients with tumors harboring mutations in KRAS are unlikely to benefit from anti-EGFR antibody therapy alone or in combination with chemotherapy (see e.g. Amado et al. 2008 J Clin On col. 2008 Apr. 1; 26(10): 1626-34, Bokemeyer et al. 2009 J Clin Oncol. 2009 Feb. 10; 27(5):663-71). One particular “hot spot” for sequence variation that may be targeted for identifying sequence variation is at position 35 of the gene. Identification of KRAS sequence variants can be used in treatment selection, such as in treatment selection for a subject with colorectal cancer.

In some embodiments, one or more sequence variants are identified in the all or part of the PIK3CA gene. Somatic mutations in PIK3CA have been frequently found in various type of cancers, for example, in 10-30% of colorectal cancers (see e.g. Samuels et al. 2004 Science. 2004 Apr. 23; 304(5670):554.). These mutations are most commonly located within two “hotspot” areas within exon 9 (the helical domain) and exon 20 (the kinase domain), which may be specifically targeted for amplification and/or analysis for the detection sequence variants. Position 3140 may also be specifically targeted.

In some embodiments, one or more sequence variants are identified in the all or part of the BRAF gene. Near 50% of all malignant melanomas have been reported as harboring somatic mutations in BRAF (see e.g. Maldonado et al., J Natl Cancer Inst. 2003 Dec. 17; 95(24):1878-90). BRAF mutations are found in all melanoma subtypes but are most frequent in melanomas derived from skin without chronic sun-induced damage. Among the most common BRAF mutations in melanoma are missense mutations V600E, which substitutes valine at position 600 with glutamine. BRAF V600E mutations are associated with clinical benefit of BRAF inhibitor therapy. Detection of BRAF mutation can be used in melanoma treatment selection and studies of the resistance to the targeted therapy.

In some embodiments, one or more sequence variants are identified in the all or part of the EGFR gene. EGFR mutations are frequently associated with Non-Small Cell Lung Cancer (about 10% in the US and 35% in East Asia; see e.g. Pao et al., Proc Natl Acad Sci US A. 2004 Sep. 7; 101(36):13306-11). These mutations typically occur within EGFR exons 18-21, and are usually heterozygous. Approximately 90% of these mutations are exon 19 deletions or exon 21 L858R point mutations.

In some embodiments, one or more sequence variants are identified in the all or part of the KIT gene. Near 85% of Gastrointestinal Stromal Tumor (GIST) have been reported as harboring KIT mutations (see e.g. Heinrich et al. 2003 J Clin Oncol. 2003 December I; 21 (23):4342-9). The majority of KIT mutations are found in juxtamembrane domain (exon 11, 70%), extracellular dimerization motif(exon 9, 10-15%), tyrosine kinase I (TKI) domain (exon 13, 1-3%), and tyrosine kinase 2 (TK2) domain and activation loop (exon 17, 1-3%). Secondary KIT mutations are commonly identified after target therapy imatinib and after patients have developed resistance to the therapy.

Additional non-limiting examples of genes associated with cancer, all or a portion of which may be analyzed for sequence variants according to a method described herein include, but are not limited to PTEN; ATM; ATR; EGFR; ERBB2; ERBB3; ERBB4; Notch1; Notch2; Notch3; Notch4; AKT; AKT2; AKT3; HIF; HIF1a; HIF3a; Met; HRG; Bcl2; PPAR alpha; PPAR gamma; WTT (Wilms Tumor); FGF Receptor Family members (5 members: 1, 2, 3, 4, 5); CDKN2a; APC; RB (retinoblastoma); MEN1; VHL; BRCAT; BRCA2; AR; (Androgen Receptor); TSG101; IGF; IGF Receptor; Igf1 (4 variants); Igf2 (3 variants); Igf 1 Receptor; Igf 2 Receptor; Bax; Bcl2; caspases family (9 members: 1, 2, 3, 4, 6, 7, 8, 9, 12); Kras; and Apc. Further examples are provided elsewhere herein. Examples of cancers that may be diagnosed based on calling one or more sequence variants in accordance with a method disclosed herein include, without limitation, Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof. Non-limiting examples of specific sequence variants associated with cancer are provided in Table 3.

TABLE 3

| | Mutation | COSMIC | |
|---|---|---|---|
| | Amino acid | mutation % in | |
| Gene | Nucleotide | melanoma | Clinical relevance |
| BRAF | V600E | 50% (80-90% among | Increase sensitivity to BRAF |
| | 1799T > A | BRAF mutations) | inhibitors |
| | | | Increase sensitivity to MEK |
| | | | inhibitors |
| BRAF | V600E | 50% (80-90% among | Increase sensitivity to BRAF |
| | 1799_1800delTGinsAA | BRAF mutations) | inhibitors |
| | | | Increase sensitivity to MEK |
| | | | inhibitors |
| BRAF | V600R | 50% (<5% among | Increase sensitivity to BRAF |
| | 1798_1799delGTinsAG | BRAF mutations) | inhibitors |
| | | | Respond to MEK inhibitors |
| BRAF | V600M | 50% (<1% among | Increase sensitivity to BRAF |
| | 1798G > A | BRAF mutations) | inhibitors |
| | | | Respond to MEK inhibitors |
| BRAF | V600K | 50% (5% among BRAF | Increase sensitivity to BRAF |
| | 1798_1799delGTinsAA | mutations) | inhibitors |
| | | | Increase sensitivity to MEK |
| | | | inhibitors |
| BRAF | V600G | 50% (<1% among | Increase sensitivity BRAF |
| | 1799T > G | BRAF mutations) | inhibitors |
| | | | Respond to MEK inhibitors |
| BRAF | V600D | 50% (<5% among | Increase sensitivity to BRAF |
| | 1799-800delTGinsAT | BRAF mutations) | inhibitors |
| | | | Respond to MEK inhibitors |
| BRAF | L597V | 50% (1% among BRAF | Respond to BRAF inhibitors |
| | 1789C > G | mutations) | Respond to MEK inhibitors |
| BRAF | L597S | 50% (<1% among | Respond to BRAF inhibitors |
| | 1789 1790delCTinsTC | BRAF mutations) | Respond to MEK inhibitors |
| BRAF | L597Q | 50% (<1% among | Respond to BRAF inhibitors |
| | 1790T > A | BRAF mutations) | Respond to MEK inhibitors |
| BRAF | L597R | 50% (<1% among | Respond to BRAF inhibitors |
| | 1790T > G | BRAF mutations) | Respond to MEK inhibitors |
| BRAF | D594N | 50% (<1% among | Not to BRAF inhibitors |
| | 1799_1780delTGinsGA | BRAF mutations) | May respond to MEK |
| | | | inhibitors |
| BRAF | D594H | 50% (<1% among | Not to BRAF inhibitors |
| | 1780C > G | BRAF mutations) | May respond to MEK |
| | | | inhibitors |
| BRAF | D594E | 50% (<1% among | Not to BRAF inhibitors |
| | 1782T > A | BRAF mutations) | May respond to MEK |
| | | | inhibitors |
| BRAF | D594E | 50% (<1% among | Not to BRAF inhibitors |
| | 1782T > G | BRAF mutations) | May respond to MEK |
| | | | inhibitors |
| BRAF | D594G | 50% (<1% among | Not to BRAF inhibitors |
| | 1781A > G | BRAF mutations) | May respond to MEK |
| | | | inhibitors |
| BRAF | D594V | 50% (<1% among | Not to BRAF inhibitors |
| | 1781A > T | BRAF mutations) | May respond to MEK |
| | | | inhibitors |
| NRAS | G12D | 13-25% (4% among | cytotoxic chemotherapy |
| | 35G > A | NRAS mutations) | |
| NRAS | G13R | 13-25% (2% among | cytotoxic chemotherapy |
| | 37G > C | NRAS mutations) | Not to BRAF inhibitors |
| | | | May respond to MEK |
| | | | inhibitors |
| NRAS | G13D | 13-25% (2% among | cytotoxic chemotherapy |
| | 38G > A | NRAS mutations) | Not to BRAF inhibitors |
| | | | May respond to MEK |
| | | | inhibitors |
| NRAS | G13V | 13-25% (2% among | cytotoxic chemotherapy |
| | 38G > T | NRAS mutations) | Not to BRAF inhibitors |
| | | | May respond to MEK |
| | | | inhibitors |
| NRAS | Q61K | 13-25% (34% among | Respond to MEK inhibitors |
| | 181C > A | NRAS mutations) | |
| NRAS | Q61L | 13-25% (8% among | Respond to MEK inhibitors |
| | 182A > T | NRAS mutations) | |
| NRAS | Q61R | 13-25% (35% among | Respond to MEK inhibitors |
| | 182A > G | NRAS mutations) | |
| NRAS | Q61H | 13-25% (2% among | Respond to MEK inhibitors |
| | 183A > C | NRAS mutations) | |
| NRAS | Q61H | 13-25% (2% among | Respond to MEK inhibitors |
| | 183A > T | NRAS mutations) | |
| CTNNB1 | S37F | ~2-3% in primary uveal | |
| | 110C > T | melanoma (46% among | |
| | | CTNNB1 mutations) | |
| CNA11 | Q209L | 34% in primary uveal | |
| | 626A > T | melanoma (92% among | |
| | | CNA11 mutations) | |
| CNA11 | Q209P | 34% in primary uveal | |
| | 626A > C | melanoma (1% among | |
| | | CNA11 mutations) | |
| GNAQ | Q209L | ~50% in primary uveal | Sensitive to MEK inhibitors |
| | 626A > T | melanoma | |
| | | (~33% among CNAQ | |
| | | mutations) | |
| GNAQ | Q209P | ~50% in primary uveal | Sensitive to MEK inhibitors |
| | 626A > C | melanoma | Sensitive to MEK inhibitors |
| | | (~64% among CNAQ | |
| | | mutations) | |
| GNAQ | Q209R | ~50% in primary uveal | |
| | 626A > G | melanoma | |
| | | (~2% among CNAQ | |
| | | mutations) | |
| KIT | K624E | ~20% among KIT | Increase sensitivity to KIT |
| | 1924A > G | mutant malignant | inhibitor |
| | | melanomas | |
| KIT | D816H | ~5% among KIT | Increase sensitivity to KIT |
| | 2446G > C | mutant malignant | inhibitor |
| | | melanomas | |
| KIT | L567P | ~25% among KIT | Increase sensitivity to KIT |
| | 1727T > C | mutant malignant | inhibitor |
| | | melanomas | |
| KIT | V559A | ~20% among KIT | Increase sensitivity to KIT |
| | 1676T > C | mutant malignant | inhibitor |
| | | melanomas | |
| KIT | V559D | ~5% among KIT | Increase sensitivity to KIT |
| | 1676T > A | mutant malignant | inhibitor |
| | | melanomas | |
| KIT | W557R | ~10% among KIT | Increase sensitivity to KIT |
| | 1669T > C | mutant malignant | inhibitor |
| | | melanomas | |
| KIT | W557R | ~10% among KIT | Increase sensitivity to KIT |
| | 1669T > A | mutant malignant | inhibitor |
| | | melanomas | |

In addition, the methods and compositions disclosed herein may be useful in discovering new, rare mutations that are associated with one or more cancer types, stages, or cancer characteristics. For example, populations of individuals sharing a characteristic under analysis (e.g. a particular disease, type of cancer, stage of cancer, etc.) may be subjected to a method of detection sequence variants according to the disclosure so as to identify sequence variants or types of sequence variants (e.g. mutations in particular genes or parts of genes). Sequence variants identified as occurring with a statistically significantly greater frequency among the group of individuals sharing the characteristic than in individuals without the characteristic may be assigned a degree of association with that characteristic. The sequence variants or types of sequence variants so identified may then be used in diagnosing or treating individuals discovered to harbor them.

Other therapeutic applications include use in non-invasive fetal diagnostics. Fetal DNA can be found in the blood of a pregnant woman. Methods and compositions described herein can be used to identify sequence variants in circulating fetal DNA, and thus may be used to diagnose one or more genetic diseases in the fetus, such as those associated with one or more causal genetic variants. Non-limiting examples of causal genetic variants are described herein, and include trisomies, cystic fibrosis, sickle-cell anemia, and Tay-Saks disease. In this embodiment, the mother may provide a control sample and a blood sample to be used for comparison. The control sample may be any suitable tissue, and will typically be processed to extract cellular DNA, which can then be sequenced to provide a reference sequence. Sequences of cfDNA corresponding to fetal genomic DNA can then be identified as sequence variants relative to the maternal reference. The father may also provide a reference sample to aid in identifying fetal sequences, and sequence variants.

Still further therapeutic applications include detection of exogenous polynucleotides, such as from pathogens (e.g. bacteria, viruses, fungi, and microbes), which information may inform a diagnosis and treatment selection. For example, some HIV subtypes correlate with drug resistance (see e.g. hivdb.stanford.edu/pages/genotype-rx). Similarly, HCV typing, subtyping and isotype mutations can also be done using the methods and compositions of the present disclosure. Moreover, where an HPV subtype is correlated with a risk of cervical cancer, such diagnosis may further inform an assessment of cancer risk. Further non-limiting examples of viruses that may be detected include Hepadnavirus hepatitis B virus (HBV), woodchuck hepatitis virus, ground squirrel (Hepadnaviridae) hepatitis virus, duck hepatitis B virus, heron hepatitis B virus, Herpesvirus herpes simplex virus (HSV) types 1 and 2, varicella-zoster virus, cytomegalovirus (CMV), human cytomegalovirus (HCMV), mouse cytomegalovirus (MCMV), guinea pig cytomegalovirus (GPCMV), Epstein-Barr virus (EBV), human herpes virus 6 (HHV variants A and B), human herpes virus 7 (HHIV-7), human herpes virus 8 (HHIV-8), Kaposi's sarcoma-associated herpes virus (KSHV), B virus Poxvirus vaccinia virus, variola virus, smallpox virus, monkeypox virus, cowpox virus, camelpox virus, ectromelia virus, mousepox virus, rabbitpox viruses, raccoonpox viruses, molluscum contagiosum virus, orf virus, milker's nodes virus, bovine papullar stomatitis virus, sheeppox virus, goatpox virus, lumpy skin disease virus, fowlpox virus, canarypox virus, pigeonpox virus, sparrowpox virus, myxoma virus, hare fibroma virus, rabbit fibroma virus, squirrel fibroma viruses, swinepox virus, tanapox virus, Yabapox virus, Flavivirus dengue virus, hepatitis C virus (HCV), GB hepatitis viruses (GBV-A, GBV-B and GBV-C), West Nile virus, yellow fever virus, St. Louis encephalitis virus, Japanese encephalitis virus, Powassan virus, tick-borne encephalitis virus, Kyasanur Forest disease virus, Togavirus, Venezuelan equine encephalitis (VEE) virus, chikungunya virus, Ross River virus, Mayaro virus, Sindbis virus, rubella virus, Retrovirus human immunodeficiency virus (HIV) types 1 and 2, human T cell leukemia virus (HTLV) types 1, 2, and 5, mouse mammary tumor virus (MMTV), Rous sarcoma virus (RSV), lentiviruses, Coronavirus, severe acute respiratory syndrome (SARS) virus, Filovirus Ebola virus, Marburg virus, Metapneumoviruses (MPV) such as human metapneumovirus (HMPV), Rhabdovirus rabies virus, vesicular stomatitis virus, Bunyavirus, Crimean-Congo hemorrhagic fever virus, Rift Valley fever virus, La Crosse virus, Hantaan virus, Orthomyxovirus, influenza virus (types A, B, and C), Paramyxovirus, parainfluenza virus (PIV types 1, 2 and 3), respiratory syncytial virus (types A and B), measles virus, mumps virus, Arenavirus, lymphocytic choriomeningitis virus, Junin virus, Machupo virus, Guanarito virus, Lassa virus, Ampari virus, Flexal virus, Ippy virus, Mobala virus, Mopeia virus, Latino virus, Parana virus, Pichinde virus, Punta toro virus (PTV), Tacaribe virus, and Tamiami virus.

Examples of bacterial pathogens that may be detected by methods of the disclosure include, without limitation, Specific examples of bacterial pathogens include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Moraxella catarrhalis, Morganella sp., Mobiluncus sp., Micrococcus sp., Mycobacterium sp. (such as Mycobacterium leprae, Mycobacterium tuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Plesiomonas shigelloides. Prevotella sp., Porphyromonas sp., Prevotella melaminogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia among others.

In some embodiments, the methods and compositions of the disclosure are used in monitoring organ transplant recipients. Typically, polynucleotides from donor cells will be found in circulation in a background of polynucleotides from recipient cells. The level of donor circulating DNA will generally be stable if the organ is well accepted, and the rapid increase of donor DNA (e.g. as a frequency in a given sample) can be used as an early sign of transplant rejection. Treatment can be given at this stage to prevent transplant failure. Rejection of the donor organ has been shown to result in increased donor DNA in blood; see Snyder et al., PNAS 108(15):6629 (2011). The present disclosure provides significant sensitivity improvements over prior techniques in this area. In this embodiment, a recipient control sample (e.g. cheek swab, etc.) and a donor control sample can be used for comparison. The recipient sample can be used to provide that reference sequence, while sequences corresponding to the donor's genome can be identified as sequence variants relative to that reference. Monitoring may comprise obtaining samples (e.g. blood samples) from the recipient over a period of time. Early samples (e.g. within the first few weeks) can be used to establish a baseline for the fraction of donor cfDNA. Subsequent samples can be compared to the baseline. In some embodiments %, an increase in the fraction of donor cfDNA of about or at least about 10%, 20%, 30%, 40%, 50%, 60%, 70%, 80%, 90%, 95%, 100%, 250%, 500%, 1000%, or more may serve as an indication that a recipient is in the process of rejecting donor tissue.

The practice of some embodiments disclosed herein employ, unless otherwise indicated, conventional techniques of immunology, biochemistry, chemistry, molecular biology, microbiology, cell biology, genomics, and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. Sequence identity, such as for the purpose of assessing percent complementarity, may be measured by any suitable alignment algorithm, including but not limited to the Needleman-Wunsch algorithm (see e.g. the EMBOSS Needle aligner available at www.ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/nucleotide.html, optionally with default settings). Optimal alignment may be assessed using any suitable parameters of a chosen algorithm, including default parameters.

### Computer Systems

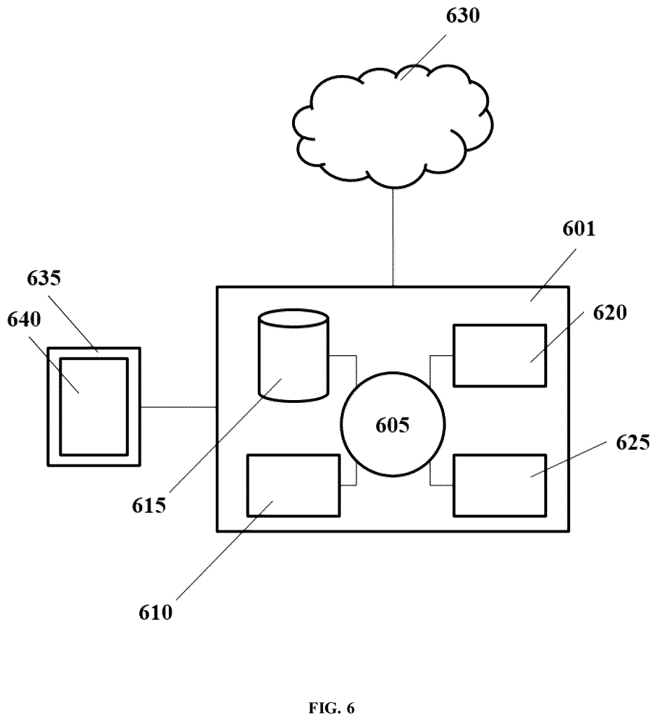

The present disclosure provides computer systems that are programmed to implement methods of the disclosure. FIG. 6 shows a computer system 601 that is programmed or otherwise configured to detect sequence variants. The computer system 601 can regulate various aspects of sequence variant detection of the present disclosure, such as, for example, circularization, rolling circle amplification, fragmentation, PCR amplification, and sequencing. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries, and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user. Examples of remote computer systems include personal computers (e.g., portable PC), slate or tablet PC's (e.g., Apple®iPad, Samsung® Galaxy Tab), telephones, Smart phones (e.g., Apple®iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, a method of detecting sequence variants. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605. The algorithm can, for example, identify sequence variants vs errors.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Variant Detection Method

A sample is obtained comprising a plurality of linear double stranded DNA molecules. The plurality of double stranded DNA molecules is denatured to create a plurality of linear single stranded DNA molecules. The plurality of linear single stranded DNA molecules is circularized to create a plurality of circularized single stranded DNA molecules. A plurality of primers is annealed to the plurality of circularized single stranded DNA molecules and the circularized single stranded DNA molecules are amplified using rolling circle amplification using a strand displacing polymerase creating a plurality of concatemers. The concatemers are subjected to second strand amplification to create double stranded concatemers. The double stranded concatemers are fragmented or sheared to create sheared concatemers having shear points. Adapters are ligated to the sheared concatemers and the adapter ligated concatemers are subjected to PCR using adapter-tailed 5′ primers that bind to the adapters and adapter-tailed 3′ primers that bind to a target sequence of the concatemers. The PCR products are subjected to sequencing and sequence differences are identified. The variant is detected only when the sequence difference occurs in multiple copies of the linear DNA found in the concatemer and in multiple concatemers having different shear points.

### Example 2: Detecting Low-Frequency Mutations from Mixture of Cell Line DNA

A cell line mixture was generated by mixing genomic DNA from 6 difference cancer cell lines with a control cell line, and fragmented to the size of mononucleosomal DNA (˜166 bp). The resulting DNA contained the following cancer specific mutations at ˜0.1-0.2% allele frequency.

TABLE 4
Cancer Specific Mutations

| Gene | Variant Name | Variant |
|---|---|---|
| KRAS | G12D | chr12: 25398284-25398285C > T |
| EGFR | L858R | chr7: 55259515-55259516T > G |
| BRAF | V600E | chr7: 140453136-140453137A > T |
| NRAS | Q61R | chr1: 115256529-115256530T > C |
| PIK3CA | H1047R | chr3: 178952085-178952086A > G |

30 ng of the fragmented DNA mix was used for each circularization reaction. DNA was denatured at 96° C. for 30 seconds, then PCR tubes were chilled on ice for 2 minutes. Ligation mix (2 μl of 10× CircLigase buffer, 4 μl 5 M betaine, 1 μl 50 mM MnCl₂, 1 μl CircLigase II) was added to each tube, and the reaction proceeded at 60° C. for 2 hours on a PCR machine. The DNA was then amplified by random priming and Phi29 polymerase. DNA samples were incubated at 30° C. for 2.5 hours followed by inactivation at 65° C. for 10 minutes. The amplification products were cleaned up using Agencourt AMPure XP Purification (1.6X) (Beckman Coulter), and then fragmented using a Covaris S220 sonicator to obtain a fragment size of approximately 400 bp. 500ng of the sonicated whole genome amplification (WGA) DNA was used for adaptor ligation and purification with KAPA Hyper Prep Kit (KK8500) according to manufacturer's protocol. After size selection and purification, 20 μl ligated product was added to 25 μl 2× KAPA HiFi Hotstart ready mix and 5 μl 10 μM P5 plus a pool of primers targeting the mutations, including KRASG12D, EGFRL858R, BRAFV600E, NRASQ61R and PIK3CAH1047R. The targets were amplified using the following cycling program: 98° C., 45 seconds; 5 cycles of (98° C., 15 seconds; 60° C., 30 seconds; 72° C., 30 seconds); 72° C., 60 seconds. The PCR products were then purified by Ampure XP beads, and then amplified further using 5 μl 10 μM P5 and P7 primers for 25 cycles. The final amplification products were purified and sequenced in a HiSeq 2500, with an average depth of 30,000×.

Sequencing data was analyzed to make variant calls. Variant calling included a step requiring that a sequence difference occur on two copies of the repeats in one read to be counted as a variant. Results for the detection of various mutations, including their frequency in the sample, are shown in Table 5.

TABLE 5
Mutation Detection Results

| | Variant | | 0.1% cell line | 0.1% cell line |
|---|---|---|---|---|
| Gene | Name | Variant | mix rep 1 | mix rep 2 |
| KRAS | G12D | chr12: 25398284-25398285C > T | 0.19% | 0.18% |
| EGFR | L858R | chr7: 55259515-55259516T > G | 0.02% | 0.07% |
| BRAF | V600E | chr7: 140453136-140453137A > T | 0.073% | 0.11% |
| NRAS | Q61R | chr1: 115256529-115256530T > C | 0.08% | 0.07% |
| PIK3CA | H1047R | chr3: 178952085-178952086A > G | 0.12% | 0.13% |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of identifying a sequence variant comprising:
(a) circularizing individual polynucleotides of a plurality of polynucleotides to form a plurality of circular polynucleotides, each circular polynucleotide having a junction between a 5′ end and a 3′ end;
(b) amplifying said plurality of circular polynucleotides of (a) to produce a plurality of amplified polynucleotides, each amplified polynucleotide having more than one copy of the circular polynucleotide;
(c) shearing said amplified polynucleotides or a derivative thereof to produce a plurality of sheared polynucleotides, each sheared polynucleotide comprising a 5′ end shear point and a 3′ end shear point;
(d) subjecting said plurality of sheared polynucleotides or a derivative thereof to sequencing to identify a plurality of sequence reads of said plurality of sheared polynucleotides;
(e) comparing said plurality of sequence reads to a reference sequence to obtain a sequence difference;
(f) calling a sequence difference as the sequence variant when the sequence difference occurs in (i) at least two copies on one sheared polynucleotide and (ii) at least two different sheared polynucleotides having different 5′ end shear points and/or 3′ end shear points.

2. The method of claim 1, further comprising subsequent to (c), attaching a first adapter to said 5′ end shear point and a second adapter to said 3′ end shear point of each of said plurality of sheared polynucleotides or a derivative thereof to create a plurality of adapter-linked sheared polynucleotides.

3. The method of claim 2, further comprising amplifying said plurality of adapter-linked sheared polynucleotides using a first primer that binds to said first adapter and a second primer that binds to said second adapter or amplifying one or more target sequences of said plurality of adapter-linked sheared polynucleotides using a first primer that binds to said first adapter and at least a second primer that binds to said one or more target sequences in said sheared polynucleotide.

4. (canceled)

5. The method of claim 1, further comprising subsequent to (c), enriching a target sequence in said plurality of sheared polynucleotides or a derivative thereof.

6. The method of claim 5, wherein enriching comprises contacting said plurality of sheared polynucleotides or a derivative thereof with a capture probe that binds to said target sequence or wherein enriching comprises amplification with at least one primer that binds to said target sequence.

7. (canceled)

8. The method of claim 1, wherein (a) comprises ligating ends of each of said plurality of polynucleotides or a derivative thereof to one another.

9. The method of claim 1, wherein (a) comprises coupling an adapter to said 5′ end, said 5′ end, or both said 5′ end and said 3′ end of each of said plurality of polynucleotides or a derivative thereof.

10. The method of claim 1, wherein (b) is effected by a polymerase having strand-displacement activity or a polymerase having 5′ to 3′ exonuclease activity.

11. (canceled)

12. The method of claim 1, wherein (b) comprises contacting said plurality of circular polynucleotides with an amplification reaction mixture comprising random primers.

13. The method of claim 1, wherein (b) comprises contacting said plurality of circular polynucleotides with an amplification mixture comprising at least one primer that hybridizes to a target sequence of at least one of said plurality of circular polynucleotides.

14. The method of claim 1, wherein said polynucleotides are single-stranded.

15. The method of claim 1, wherein said polynucleotides are double-stranded.

16. The method of claim 1, wherein said polynucleotides are cell-free polynucleotides.

17. The method of claim 1, wherein said polynucleotides are deoxyribonucleic acid (DNA), ribonucleic acid (RNA), or a combination thereof.

18. The method of claim 1, wherein said polynucleotides are from a tumor.

19. The method of claim 1, wherein (d) comprises (i) bringing said plurality of sheared polynucleotides or a derivative thereof in contact with a plurality of nucleotides in the presence of a polymerase to incorporate one or more nucleotides of said plurality of nucleotides into a growing strand complementary to a strand of said sheared polynucleotides or derivative thereof, and (ii) detecting one or more signals indicative of incorporation of said one or more nucleotides into said growing strand.

20. (canceled)

21. The method of claim 1, wherein said sequence variant comprises a single nucleotide variant, a fusion, an insertion, a deletion, or an epigenetic modification.

22. The method of claim 1, wherein said sequence variant is indicative of minimum residual disease (MRD).

23. The method of claim 1, wherein said polynucleotides are from a bodily fluid comprising urine, saliva, blood, serum, or plasma.

24. (canceled)

25. The method of claim 1, further comprising detecting minimum residual disease (MRD).
